SEQUENCE LISTING (6 sequences)

SEQ ID NO: 1 - DNA, 2211 bp, Artificial Sequence
  Description of Artificial Sequence Synthetic polynucleotide
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaagcctacgaccagcagctcaaagcgggtgacaatccgtacctgcggtataatcacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcagtcgccacaagagccagactcctcctcgggcatcggcaagacaggccagcagcccgctaaaaagagactcaattttggtcagactggcgactcagagtcagtccccgacccacaacctctcggagaacctccagcagccccctcaggtctgggacctaatacaatggcttcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacggcacctcgggaggaagcaccaacgacaacacctattttggctacagcaccccctgggggtattttgacttcaacagattccactgtcacttttcaccacgtgactggcaacgactcatcaacaacaattggggattccggcccaaaagactcaacttcaagctgttcaacatccaggtcaaggaagtcacgacgaacgaaggcaccaagaccatcgccaataatctcaccagcaccgtgcaggtctttacggactcggagtaccagttaccgtacgtgctaggatccgctcaccagggatgtctgcctccgttcccggcggacgtcttcatggttcctcagtacggctatttaactttaaacaatggaagccaagccctgggacgttcctccttctactgtctggagtatttcccatcgcagatgctgagaaccggcaacaactttcagttcagctacaccttcgaggacgtgcctttccacagcagctacgcgcacagccagagcctggacaggctgatgaatcccctcatcgaccagtacctgtactacctggtcagaacgcaaacgactggaactggagggacgcagactctggcattcagccaagcgggtcctagctcaatggccaaccaggctagaaattgggtgcccggaccttgctaccggcagcagcgcgtctccacgacaaccaaccagaacaacaacagcaactttgcctggacgggagctgccaagtttaagctgaacggccgagactctctaatgaatccgggcgtggcaatggcttcccacaaggatgacgacgaccgcttcttcccttcgagcggggtcctgatttttggcaagcaaggagccgggaacgatggagtggattacagccaagtgctgattacagatgaggaagaaatcaaggctaccaaccccgtggccacagaagaatatggagcagtggccatcaacaaccaggccgccaatacgcaggcgcagaccggactcgtgcacaaccagggggtgattcccggcatggtgtggcagaatagagacgtgtacctgcagggtcccatctgggccaaaattcctcacacggacggcaactttcacccgtctcccctgatgggcggctttggactgaagcacccgcctcctcaaattctcatcaagaacacaccggttccagcggacccgccgcttaccttcaaccaggccaagctgaactctttcatcacgcagtacagcaccggacaggtcagcgtggaaatcgagtgggagctgcagaaagaaaacagcaaacgctggaatccagagattcaatacacttccaactactacaaatctacaaatgtggactttgctgtcaacacggagggggtttatagcgagcctcgccccattggcacccgttacctcacccgcaacctgtaa

SEQ ID NO: 2 - DNA, 7465 bp, Artificial Sequence
  Description of Artificial Sequence Synthetic polynucleotide
atgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcagccccctcaggtctgggacctaatacaatggcttcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacggcacctcgggaggaagcaccaacgacaacacctattttggctacagcaccccctgggggtattttgacttcaacagattccactgtcacttttcaccacgtgactggcaacgactcatcaacaacaattggggattccggcccaaaagactcaacttcaagctgttcaacatccaggtcaaggaagtcacgacgaacgaaggcaccaagaccatcgccaataatctcaccagcaccgtgcaggtctttacggactcggagtaccagttaccgtacgtgctaggatccgctcaccagggatgtctgcctccgttcccggcggacgtcttcatggttcctcagtacggctatttaactttaaacaatggaagccaagccctgggacgttcctccttctactgtctggagtatttcccatcgcagatgctgagaaccggcaacaactttcagttcagctacaccttcgaggacgtgcctttccacagcagctacgcgcacagccagagcctggacaggctgatgaatcccctcatcgaccagtacctgtactacctggtcagaacgcaaacgactggaactggagggacgcagactctggcattcagccaagcgggtcctagctcaatggccaaccaggctagaaattgggtgcccggaccttgctaccggcagcagcgcgtctccacgacaaccaaccagaacaacaacagcaactttgcctggacgggagctgccaagtttaagctgaacggccgagactctctaatgaatccgggcgtggcaatggcttcccacaaggatgacgacgaccgcttcttcccttcgagcggggtcctgatttttggcaagcaaggagccgggaacgatggagtggattacagccaagtgctgattacagatgaggaagaaatcaaggctaccaaccccgtggccacagaagaatatggagcagtggccatcaacaaccaggccgccaatacgcaggcgcagaccggactcgtgcacaaccagggggtgattcccggcatggtgtggcagaatagagacgtgtacctgcagggtcccatctgggccaaaattcctcacacggacggcaactttcacccgtctcccctgatgggcggctttggactgaagcacccgcctcctcaaattctcatcaagaacacaccggttccagcggacccgccgcttaccttcaaccaggccaagctgaactctttcatcacgcagtacagcaccggacaggtcagcgtggaaatcgagtgggagctgcagaaagaaaacagcaaacgctggaatccagagattcaatacacttccaactactacaaatctacaaatgtggactttgctgtcaacacggagggggtttatagcgagcctcgccccattggcacccgttacctcacccgcaacctgtaattacgtgttaatcaataaaccggttgattcgtttcagttgaactttggtgtcgcggccgctcgataagcttttgttccctttagtgagggttaattccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgaggaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccctagagtcctgtattagaggtcacgtgagtgttttgcgacattttgcgacaccatgtggtcacgctgggtatttaagcccgagtgagcacgcagggtctccattttgaagcgggaggtttgaacgcgcagccgcc

SEQ ID NO: 3 - DNA, 19 bp, Artificial Sequence
  Description of Artificial Sequence Synthetic primer
cctcgactgtgccttctag

SEQ ID NO: 4 - DNA, 22 bp, Artificial Sequence
  Description of Artificial Sequence Synthetic primer
ccccagaatagaatgacaccta

SEQ ID NO: 5 - DNA, 24 bp, Artificial Sequence
  Description of Artificial Sequence Synthetic probe
gccactcccactgtcctttcctaa

SEQ ID NO: 6 - DNA, 28 bp, Artificial Sequence
  Description of Artificial Sequence Synthetic probe
aaaatgaggaaattgcatcgcattgtct

(19) United States
(12) Patent Application Publication
(10) Pub. No.: US 2013/0090374 A1
(43) Pub. Date: Apr. 11, 2013

(54) METHODS FOR THE TREATMENT OF TAY-SACHS DISEASE, SANDHOFF DISEASE, AND GM1-GANGLIOSIDOSIS

(71) Applicants: Miguel Sena-Esteves, Westford, MA (US); Timothy M. Cox, Cambridge (GB); Maria Begona Cachon-Gonzalez, Saffron Walden (GB); Thomas N. Seyfried, Foxboro, MA (US)

(72) Inventors: Miguel Sena-Esteves, Westford, MA (US); Timothy M. Cox, Cambridge (GB); Maria Begona Cachon-Gonzalez, Saffron Walden (GB); Thomas N. Seyfried, Foxboro, MA (US)

(21) Appl. No.: 13/452,339

(22) Filed: Apr. 20, 2012

Related U.S. Application Data
(60) Provisional application No. 61/477,504, filed on Apr. 20, 2011.

(51) Int. Cl. A61K 48/00; A61K 38/47

(52) U.S. Cl. CPC A61K 48/0083; A61K 38/47
USPC 514/44 R

(57) ABSTRACT

The present disclosure provides methods for the treatment of lysosomal storage disorders using gene replacement therapy. In particular, methods are provided for the treatment of Tay-Sachs disease, Sandhoff Disease, and GM1-gangliosidosis using enzyme replacement therapy. Expression constructs encoding enzymes required for ganglioside metabolism are delivered to the brain of subjects with an enzyme deficiency. Methods are also provided for delaying the onset of, reducing the likelihood of onset of, or reducing the severity of Tay-Sachs disease, Sandhoff Disease, and GM1-gangliosidosis.

### RELATED APPLICATIONS

This application claims the benefit under 37 U.S.C. §119(e) of U.S. Provisional Application Ser. No. 61/477,504, filed Apr. 20, 2011, the entire contents of which are incorporated herein by reference.

### FIELD OF INVENTION

The present technology relates to generally the treatment of lysosomal storage disorders. In particular, the present technology relates to methods of treating Tay-Sachs Disease, Sandhoff Disease, and GM-gangliosidosis using gene replacement therapy.

### BACKGROUND

The following description is provided to assist the understanding of the reader. None of the information provided or references cited is admitted to be prior art to the present invention.

Lysosomal storage diseases (LSDs) comprise a family of more than forty distinct human and animal diseases resulting from defects of lysosomal degradative enzymes and subsequent accumulation of undegraded substrates in lysosomes of various cell types. As a group, LSDs are the most common type of childhood genetic disorder, with an estimated combined frequency of 1 in 7700 live births, and thus represent a significant worldwide health problem. It is estimated that at least 60% of all LSDs involve the central nervous system (CNS).

GM1-gangliosidosis is a neurodegenerative lysosomal storage disease caused by deficiency of acid β-galactosidase (βgal) leading to progressive accumulation of GM1-ganglioside in the CNS (FIG. 1). Age of onset of the symptoms ranges from infancy to adulthood and the severity of the clinical manifestations mostly correlates with the levels of residual enzyme activity. In the most severe form of this disease (Infantile or Type I) biochemical and neuropathological alterations have been documented in utero. Progressive neurologic deterioration, macular cherry red spot, facial dysmorphism, hepatosplenomegaly, generalized skeletal dysplasia and early death are common features of the disease.

Currently there is no effective treatment for GM1-gangliosidosis in children, although numerous therapeutic modalities have been implemented in GM1 mice with somewhat encouraging results.

Tay-Sachs and Sandhoff Diseases comprise a subset of neuronopathic LSDs characterized by storage of GM2 ganglioside in the CNS. These ‘GM2 gangliosidoses’ result from inherited defects in the lysosomal glycohydrolase, β-N-acetylhexosaminidase (Hex). The prevalence of Tay-Sachs disease (TSD) and Sandhoff disease (SD) in the general population is ˜1 in 100,000 live births for each disease, while carrier frequency in the general population is 1 in 167 for TSD and 1 in 278 for SD. However, carrier frequencies may be 100-fold higher in certain ethnic groups, such as Ashkenazi Jews, Cajuns in southern Louisiana, French Canadians in eastern Quebec, or the Pennsylvania Dutch. Having very similar clinical phenotypes in humans, TSD and SD are characterized by relentlessly progressive nervous system dysfunction and are classified according to disease severity as (1) infantile, (2) juvenile or (3) adult-onset forms. Babies with the infantile (most severe) form develop normally for the first 3-6 months of life, after which development slows and then begins to regress. A stereotypical “cherry red spot” is evident on the fundus of the retina, created by retinal neurons grossly swollen with ganglioside storage material. By age two, affected children suffer from frequent seizures, swallowing difficulties, respiratory infections, and loss of motor control. Death typically occurs before the fifth birthday. Late-onset GM2 gangliosidosis is the second most common form, but because early symptoms are common to other diseases, affected adults may be misdiagnosed for >10 years. Symptoms typically include speech difficulties, muscle weakness, tremor and ataxia. Manic-depressive or psychotic episodes are present in about 30% of affected persons. The majority of late-onset patients are wheelchair-bound by age 30-40. The juvenile forms vary greatly in severity from case to case, but all juvenile forms of GM2 gangliosidosis are fatal. Although GM2 gangliosidosis was first described more than a century ago, it remains largely untreatable.

β-Hexosaminidase is composed of 2 subunits, α and β, encoded respectively by the HEXA and HEXB genes. HEXA mutations cause TSD, while defects in HEXB produce SD, both resulting in abnormal storage of GM2 ganglioside in the CNS. GM2 ganglioside is degraded by the coordinated action of 3 gene products: the α and β subunits of hexosaminidase and the GM2 activator protein, a non-degradative accessory protein necessary for ganglioside presentation to the Hex enzyme. Hex subunits dimerize to form separate isozymes with different substrate specificities: HexA (αβ), HexB (ββ) and HexS (αα) (an unstable isoform present at very low levels). Only those isozymes containing the α-subunit are capable of appreciable GM2-ganglioside degradation. Therefore, HexA is the predominant isozyme responsible for clearance of GM2 ganglioside, and its function may be eliminated by defects in either the α- or β-subunit. The α- and β-subunit precursor proteins are translated and translocated into the lumen of the endoplasmic reticulum (ER), where they dimerize to form immature HexA and HexB molecules. A pool of excess α-subunit monomer is maintained for at least 5 hours in the ER and thought to force formation of the less stable HexA (αβ) isozyme through mass action, since the ββ homodimer (HexB) is more stable and more readily formed. If subunit dimerization does not occur, monomers appear to be retained in the ER and degraded. Final acquisition of the mannose-6-phosphate signal for lysosomal targeting occurs in the Golgi, and proteolytic processing to the mature isozymes occurs in the lysosome. Therefore, subunit dimerization in the ER is a first step toward ultimate isozyme maturation in the lysosome, for enzymatic activity toward GM2 ganglioside.

Gene therapy approaches for GM2-gangliosidoses take into account that simple overexpression of α- or β-subunits individually will create an imbalance in the intracellular stoichiometry of α- and β-subunits. Gene transfer experiments in cell culture have shown that overexpression of human α-subunit in human or mouse Tay-Sachs fibroblasts produces a significant reduction in HexB activity, presumably by depletion of the endogenous β-subunit pool. Also gene transfer experiments in Tay-Sachs mice have shown that co-transduction with two viral vectors encoding human α- and β-subunit separately to achieve high-level HexA synthesis and secretion. Therefore effective gene therapy strategies for GM2-gangliosidoses should utilize gene delivery vehicles encoding both the α- and β-subunits.

Although TSD was first described in 1881 and the precise enzymatic deficiency was identified in the late 1960's, it remains untreatable today. However, a number of observations have been made which encourage continuing efforts to develop effective therapy for lysosomal diseases such as TSD and SD. For example, tissues of individuals heterozygous for the gangliosidoses, who have no clinical signs of disease, can have as little as 15-20% of normal tissue enzyme activity, and patients with late onset disease and reduced severity of clinical signs have only 1-5% of normal enzyme activity, indicating that restoration of minimal functional enzyme activity may be adequate to prevent or reduce disease severity. Neufeld and coworkers first demonstrated that lysosomal enzymes secreted from normal cells are endocytosed by mutant cells with correction of the metabolic defect, suggesting that enzyme donor cells which constitute only a portion of an organ might effect restoration of lysosomal function in a larger population of cells. Discovery of this “cross-correction” mechanism in the mid 1970s stimulated the development of methods to replace missing enzymes in the various LSDs. This mechanism is the basis for all existing therapies for LSDs, including enzyme replacement therapy (ERT), which has been approved for treating a number of LSDs.

In humans, ERT has proven ineffective to treat the brain in LSDs with neurological features because the blood-brain barrier (BBB) restricts entry of peripherally infused enzymes into the brain. One of the promising experimental approaches to treat neuronopathic LSDs center on gene therapy. Adeno-associated virus (AAV) vectors have become the vectors of choice for gene delivery to the brain because of their exceptional efficiency in transducing neurons where they promote long-term expression of therapeutic genes with no apparent toxicity, and limited inflammation at the site of injection. Direct infusion of adeno-associated virus (AAV) vectors encoding lysosomal enzymes into the brain parenchyma has emerged as a viable strategy to create an in situ source of normal enzyme in the brain.

However, one obstacle to translation of the promising results obtained in animal models is the number of injections that may be needed to achieve global distribution of enzyme throughout the human brain. Based on studies in α-mannosidosis cats, it has been estimated that 40-60 injections of AAV vector may be necessary to obtain global distribution of lysosomal enzymes in the infant brain. This large number of injections makes the treatment extremely invasive with obvious risks. Therefore, alternative strategies are needed.

### SUMMARY

In some embodiments, the present disclosure provides a method for enhancing β-N-acetylhexosaminidase activity in a subject in need thereof, comprising: (a) administering to the subject a therapeutically effective amount of a composition comprising a first expression construct and a second expression construct, wherein (i) the first construct expresses the β-N-acetylhexosaminidase αsubunit; and (ii) the second construct expresses the β-N-acetylhexosaminidase βsubunit.

In some embodiments, the subject is a human predisposed to having, suspected of having, or diagnosed as having a β-N-acetylhexosaminidase deficiency. In another embodiment, the β-N-acetylhexosaminidase deficiency comprises a lysosomal storage disorder. In another embodiment, the β-N-acetylhexosaminidase deficiency comprises Tay-Sachs Disease or Sandhoff Disease. In another embodiment, the β-N-acetylhexosaminidase deficiency comprises a partial or complete loss of endogenous expression or function of the β-N-acetylhexosaminidase α subunit, β subunit, or both. In some embodiments, the β-N-acetylhexosaminidase expression constructs comprise the adeno-associated virus (AAV) vector AAVrh.8 or derivatives thereof.

In some embodiments, the method comprises administering to a subject in need thereof a therapeutically effective amount of a composition comprising a β-N-acetylhexosaminidase expression construct, wherein a single construct encodes both the α and β subunits of (β-N-acetylhexosamimidase.

In some embodiments, the method comprises administering the (β-N-acetylhexosaminidase composition to the brain of the subject. In some embodiments, the method comprises administering the composition to one or more areas of the brain selected from the group consisting of the thalamus, striatum, deep cerebellar nuclei, ventral tegmental area, and lateral ventricles. In some embodiments, the method comprises administering the composition to the brain of the subject unilaterally or bilaterally.

In some embodiments, the method comprises evaluating β-N-acetylhexosaminidase activity in the subject after administration of the composition. In some embodiments, evaluating β-N-acetylhexosaminidase activity comprises an assessment of symptoms associated with a β-N-acetylhexosaminidase deficiency. In some embodiments, evaluating β-N-acetylhexosaminidase activity comprises a biochemical assessment of β-N-acetylhexosaminidase activity. In some embodiments, the method comprises administering additional amounts of the β-N-acetylhexosaminidase composition to the subject as needed to achieve or maintain enhanced β-N-acetylhexosaminidase activity.

In another embodiment, the present disclosure provides a method for treating Tay-Sachs Disease or Sandhoff Disease comprising: (a) administering to a subject in need thereof a therapeutically effective amount of a composition comprising a first expression construct and a second expression construct, wherein (i) the first construct expresses the β-N-acetylhexosaminidase α subunit; and (ii) the second construct expresses the β-N-acetylhexosaminidase β subunit.

In some embodiments, the subject is a human predisposed to having, suspected of having, or diagnosed as having Tay-Sachs Disease or Sandhoff Disease. In some embodiments, Tay-Sachs Disease or Sandhoff Disease comprise a partial or complete loss of endogenous expression or function of the β-N-acetylhexosaminidase α subunit, β subunit, or both. In some embodiments, the expression constructs comprise the adeno-associated virus (AAV) vector AAVrh.8 or derivatives thereof.

In some embodiments, the method comprises administering the β-N-acetylhexosaminidase composition to the brain of the subject. In some embodiments, the method comprises administering the composition to one or more areas of the brain selected from the group consisting of the thalamus, striatum, deep cerebellar nuclei, ventral tegmental area, and lateral ventricles. In some embodiments, the method comprises administering the composition to the brain of the subject unilaterally or bilaterally.

In some embodiments, the method comprises evaluating β-N-acetylhexosaminidase activity in the subject after administration of the composition. In some embodiments, evaluating β-N-acetylhexosaminidase activity comprises an assessment of symptoms associated with Tay-Sachs Disease or Sandhoff Disease. In some embodiments, evaluating β-N-acetylhexosaminidase activity comprises a biochemical assessment of β-N-acetylhexosaminidase activity. In some embodiments, the method comprises administering additional amounts of the composition to the subject as needed to achieve or maintain treatment of Tay-Sachs Disease or Sandhoff Disease.

In another embodiment, the present disclosure provides a method for reducing the likelihood of onset or severity of Tay-Sachs Disease or Sandhoff Disease comprising: (a) administering to a subject in need thereof a therapeutically effective amount of a composition comprising a first expression construct and a second expression construct, wherein (i) the first construct expresses the β-N-acetylhexosaminidase α subunit; and (ii) the second construct expresses the β-N-acetylhexosaminidase β subunit. In some embodiments, the subject is a human predisposed to having, suspected of having, or diagnosed as having Tay-Sachs Disease or Sandhoff Disease. In some embodiments, Tay-Sachs Disease or Sandhoff Disease comprise a partial or complete loss of endogenous expression or function of the β-N-acetylhexosaminidase α subunit, β subunit, or both.

In some embodiments, the β-N-acetylhexosaminidase expression constructs comprise the adeno-associated virus (AAV) vector AAVrh.8 or derivatives thereof. In some embodiments, the method comprises administering the composition to the brain of the subject. In some embodiments, the method comprises administering the composition to one or more areas of the brain selected from the group consisting of the thalamus, striatum, deep cerebellar nuclei, ventral tegmental area, and lateral ventricles. In some embodiments, the method comprises administering the composition to the brain of the subject unilaterally or bilaterally.

In some embodiments, the method comprises evaluating β-N-acetylhexosaminidase activity in the subject after administration of the β-N-acetylhexosaminidase composition. In some embodiments, evaluating β-N-acetylhexosaminidase activity comprises an assessment of symptoms associated with Tay-Sachs Disease or Sandhoff Disease. In some embodiments, evaluating β-N-acetylhexosaminidase activity comprises a biochemical assessment of β-N-acetylhexosaminidase activity. In some embodiments, the method comprises administering additional amounts of the composition to the subject as needed to reduce the likelihood or severity of onset of Tay-Sachs Disease or Sandhoff Disease.

In another embodiment, the present disclosure provides a method for achieving widespread distribution of exogenous β-N-acetylhexosaminidase in the brain of a subject in need thereof, comprising: (a) administering to the brain of the subject an effective amount of a composition comprising a first expression construct and a second expression construct, wherein (i) the first construct expresses the β-N-acetylhexosaminidase α subunit; and (ii) the second construct expresses the β-N-acetylhexosaminidase β subunit. In some embodiments, the subject is a human predisposed to having, suspected of having, or diagnosed as having a β-N-acetylhexosaminidase deficiency. In some embodiments, the β-N-acetylhexosaminidase deficiency comprises a lysosomal storage disorder. In some embodiments, the β-N-acetylhexosaminidase deficiency comprises Tay-Sachs Disease or Sandhoff Disease. In some embodiments, the β-N-acetylhexosaminidase deficiency comprises a partial or complete loss of endogenous expression or function of the β-N-acetylhexosaminidase α subunit, β subunit, or both.

In some embodiments, the β-N-acetylhexosaminidase expression constructs comprise the adeno-associated virus (AAV) vector AAVrh.8 or derivatives thereof. In some embodiments, the method comprises administering the composition to one or more areas of the brain selected from the group consisting of the thalamus, striatum, deep cerebellar nuclei, ventral tegmental area, and lateral ventricles. In some embodiments, the method comprises administering the composition to the brain of the subject unilaterally or bilaterally.

In some embodiments, the method comprises evaluating β-N-acetylhexosaminidase activity in the subject after administration of the β-N-acetylhexosaminidase composition. In some embodiments, evaluating β-N-acetylhexosaminidase activity comprises an assessment of symptoms associated with a β-N-acetylhexosaminidase deficiency. In some embodiments, wherein evaluating β-N-acetylhexosaminidase activity comprises a biochemical assessment of β-N-acetylhexosaminidase activity. In some embodiments, the method comprises administering additional amounts of the composition to the subject as needed to achieve or maintain widespread exogenous β-N-acetylhexosaminidase activity in the brain of the subject.

In another embodiment, the present disclosure provides a method for enhancing β-N-acetylhexosaminidase activity in a subject in need thereof comprising: (a) evaluating β-N-acetylhexosaminidase activity in a subject administered a composition comprising a first expression construct and a second expression construct, wherein (i) the first construct expresses the β-N-acetylhexosaminidase α subunit; and (ii) the second construct expresses the β-N-acetylhexosaminidase β subunit. In some embodiments, evaluating β-N-acetylhexosaminidase activity comprises an assessment of symptoms associated with a β-N-acetylhexosaminidase deficiency. In some embodiments, evaluating β-N-acetylhexosaminidase activity comprises a biochemical assessment of β-N-acetylhexosaminidase activity.

In some embodiments, the present disclosure provides a method for enhancing acid β-galactosidase activity in a subject in need thereof, comprising: administering to the subject a therapeutically effective amount of a composition comprising an acid β-galactosidase expression construct.

In some embodiments, the subject is a human predisposed to having, suspected of having, or diagnosed as having an acid β-galactosidase deficiency. In another embodiment, the acid β-galactosidase deficiency comprises a lysosomal storage disorder. In another embodiment, the β-acid β-galactosidase deficiency comprises GM1-gangliosidosis. In another embodiment, the acid β-galactosidase deficiency comprises a partial or complete loss of endogenous expression or function of acid β-galactosidase. In some embodiments, the acid β-galactosidase expression construct comprises the adeno-associated virus (AAV) vector AAVrh.8 or derivatives thereof.

In some embodiments, the method comprises administering the acid β-galactosidase composition to the brain of the subject. In some embodiments, the method comprises administering the acid β-galactosidase composition to one or more areas of the brain selected from the group consisting of the thalamus, striatum, deep cerebellar nuclei, ventral tegmental area, and lateral ventricles. In some embodiments, the method comprises administering the composition to the brain of the subject unilaterally or bilaterally.

In some embodiments, the method comprises evaluating acid β-galactosidase activity in the subject after administration of the composition. In some embodiments, evaluating acid β-galactosidase activity comprises an assessment of symptoms associated with an acid β-galactosidase deficiency. In some embodiments, evaluating acid β-galactosidase activity comprises a biochemical assessment of β-N-acetylhexosaminidase activity. In some embodiments, the method comprises administering additional amounts of the acid β-galactosidase composition to the subject as needed to achieve or maintain enhanced acid β-galactosidase activity.

In another embodiment, the present disclosure provides a method for treating GM1-gangliosidosis in a subject in need thereof, comprising: administering to the subject a therapeutically effective amount of a composition comprising an acid β-galactosidase expression construct.

In some embodiments, the subject is a human predisposed to having, suspected of having, or diagnosed as having GM1-gangliosidosis. In some embodiments, GM1-gangliosidosis comprises a partial or complete loss of endogenous expression or function of acid β-galactosidase. In some embodiments, the acid β-galactosidase expression construct comprises the adeno-associated virus (AAV) vector AAVrh.8 or derivatives thereof.

In some embodiments, the method comprises administering the acid β-galactosidase composition to the brain of the subject. In some embodiments, the method comprises administering the composition to one or more areas of the brain selected from the group consisting of the thalamus, striatum, deep cerebellar nuclei, ventral tegmental area, and lateral ventricles. In some embodiments, the method comprises administering the composition to the brain of the subject unilaterally or bilaterally.

In some embodiments, the method comprises evaluating acid β-galactosidase activity in the subject after administration of the composition. In some embodiments, evaluating acid β-galactosidase activity comprises an assessment of symptoms associated with GM1-gangliosidosis. In some embodiments, evaluating acid β-galactosidase activity comprises a biochemical assessment of acid β-galactosidase activity. In some embodiments, the method comprises administering additional amounts of the composition to the subject as needed to achieve or maintain treatment of GM1-gangliosidosis.

In another embodiment, the present disclosure provides a method for reducing the likelihood of onset or severity of GM1-gangliosidosis in a subject in need thereof, comprising: administering to the subject a therapeutically effective amount of a composition comprising an acid β-galactosidase expression construct.

In some embodiments, the subject is a human predisposed to having, suspected of having, or diagnosed as having GM1-gangliosidosis. In some embodiments, GM1 gangliosidosis comprises a partial or complete loss of endogenous expression or function of acid β-galactosidase.

In some embodiments, the acid β-galactosidase expression construct comprises the adeno-associated virus (AAV) vector AAVrh.8 or derivatives thereof. In some embodiments, the method comprises administering the acid β-galactosidase composition to the brain of the subject. In some embodiments, the method comprises administering the composition to one or more areas of the brain selected from the group consisting of the thalamus, striatum, deep cerebellar nuclei, ventral tegmental area, and lateral ventricles. In some embodiments, the method comprises administering the composition to the brain of the subject unilaterally or bilaterally.

In some embodiments, the method comprises evaluating acid β-galactosidase activity in the subject after administration of the acid β-galactosidase composition. In some embodiments, evaluating acid β-galactosidase activity comprises an assessment of symptoms associated with GM1-gangliosidosis. In some embodiments, evaluating acid β-galactosidase activity comprises a biochemical assessment of acid β-galactosidase activity. In some embodiments, the method comprises administering additional amounts of the composition to the subject as needed to reduce the likelihood or severity of onset of GM1-gangliosidosis.

In another embodiment, the present disclosure provides a method for achieving widespread distribution of exogenous acid β-galactosidase in the brain of a subject in need thereof, comprising: administering to the subject a therapeutically effective amount of a composition comprising an acid β-galactosidase expression construct.

In some embodiments, the subject is a human predisposed to having, suspected of having, or diagnosed as having an acid β-galactosidase deficiency. In some embodiments, the acid β-galactosidase deficiency comprises a lysosomal storage disorder. In some embodiments, the acid β-galactosidase deficiency comprises GM1-gangliosidosis. In some embodiments, the acid β-galactosidase deficiency comprises a partial or complete loss of endogenous expression or function of acid β-galactosidase.

In some embodiments, the acid β-galactosidase expression construct comprises the adeno-associated virus (AAV) vector AAVrh.8 or derivatives thereof. In some embodiments, the method comprises administering the composition to one or more areas of the brain selected from the group consisting of the thalamus, striatum, deep cerebellar nuclei, ventral tegmental area, and lateral ventricles. In some embodiments, the method comprises administering the composition to the brain of the subject unilaterally or bilaterally.

In some embodiments, the method comprises evaluating acid β-galactosidase activity in the subject after administration of the acid β-galactosidase. In some embodiments, evaluating acid β-galactosidase activity comprises an assessment of symptoms associated with an acid β-galactosidase deficiency. In some embodiments, evaluating acid β-galactosidase activity comprises a biochemical assessment of acid β-galactosidase activity. In some embodiments, the method comprises administering additional amounts of the composition to the subject as needed to achieve or maintain widespread exogenous acid β-galactosidase activity in the brain of the subject.

In another embodiment, the present disclosure provides a method for enhancing acid β-galactosidase activity in a subject in need thereof, comprising: evaluating acid β-galactosidase activity in a subject administered a composition comprising an acid β-galactosidase expression construct. In some embodiments, evaluating acid β-galactosidase activity comprises an assessment of symptoms associated with an acid β-galactosidase deficiency. In some embodiments, evaluating acid β-galactosidase activity comprises a biochemical assessment of acid β-galactosidase activity.

### BRIEF DESCRIPTION OF THE FIGURES

FIG. 1. Pathways of Ganglioside Catabolism.

Ganglioside catabolic pathways are depicted schematically, beginning with the hydrolysis of GM1 to GM2 by β-galactosidase (βgal). GM2 ganglioside may be degraded by the classic (humans and mice) or alternative (mice only) pathways. In the classic pathway, GM2 is degraded to GM3 by HexA and the GM2 activator protein (GM2a). In the alternative pathway, GA2 is degraded to lactosylceramide (LacCer) by HexA (major activity) or HexB (minor activity) in the presence of GM2a. Degradation to glucosylceramide (GlcCer) and finally to ceramide (not shown) occurs in both pathways. Diagram reproduced from the cited reference.

FIG. 2. Gross Morphology of Mouse, Cat and Human Brains.

In terms of size and complexity, the cat brain is intermediate to mouse and human brains and provides a more faithful model of vector delivery and distribution challenges for CNS gene therapy in humans. Images are shown to illustrate differences in complexity and are not scaled proportionally to actual size. Approximate brain weights: mouse, 0.4 g; cat, 30 g; human infant, 400 g; human adult, 1400 g. Brain images from Comparative Mammalian Brain Collection.

FIG. 3. Distribution of βgal in the Brain Following Intrathalamic Delivery of an AAVrh.8-βgal Expression Construct.

One μl of an AAV2/8-βgal (6.13×10¹³ gc/ml) was injected into the left thalamus of 6-8 week-old GM1-gangliosidosis mice. βgal expression and distribution throughout the brain was evaluated at 4 weeks post-injection by X-gal histochemistry. Scale bars=1 mm.

FIG. 4. β-gal Distribution Outside the Cerebrum.

Cerebellum in uninjected (A) and injected (B) GM1-gangliosidosis mice. Arrowhead in (B) indicates the inferior colliculus, and arrow indicates the brain stem. βgal was absent in the left eye (C) but it was present in the ganglion cell layer (GCL) in the right eye (D). In the spinal cord (E, F)β-gal activity was found in the ascending sensorimotor pathway (arrow in E) and in cells in the dorsal horn (arrow in F). Scale bars in A, B=1 mm; Magnifications: C, D, F-200×; E-40×.

FIG. 5. Lysosomal Storage in the Brain at 2 Weeks Post-Injection.

Unesterified cholesterol storage in the brains of AAVrh.8-treated (A-F) and untreated (G-I) GM1-gangliosidosis mice was assessed by Filipin staining (blue). In the ipsilateral hemisphere of AAVrh.8-treated mice there was considerable reduction in lysosomal storage (A-C), while in the contralateral hemisphere (D-F) storage levels appeared comparable to those found in control untreated mice (G-I). Nuclei were counterstained with TO-PRO3 (red). Scale bar=200 μm.

FIG. 6. Biochemical Quantification of GM1-Ganglioside in the Cerebral Cortex at 4 Months Post-AAV Treatment.

(A) HPTLC of cortical gangliosides; (B) Quantitative analysis of total gangliosides and GM1-ganglioside content.

FIG. 7. Effect of AAV-Treatment on Motor Performance of GM1 Mice.

(A) Rotarod testing was performed prior to injection (0 months), and then at 1, 2.5, 4, and 6 months post-injection in AAV-T GM1 mice (•), AAV-TC GM1 (X), untreated GM1 (▴), and HZ mice (♦). Open-field testing measured (B) locomotor and (C) rearing activity at 2.5 (2.5M) and 4 (4M) months post-injection in HZ (white bars), untreated GM1 (black bars), AAV-T GM1 (light gray bars), and AAV-TC GM1 (dark gray bars) mice. Group sizes: n=20-24 for 0 and 1 month time points; n=14-18 for 2.5 and 4 month time points; n=10-12 for 6 month time point. Graphs represent the mean for each group at the specified time point. Error bars correspond to 1 SEM. *p<0.05 in paired Student's t-test.

FIG. 8. Effect of AAV Treatment on Visual Function in GM1 Mice.

Visual evoked potentials were measured in (A) wild type, (B) HZ, (C) untreated GM1, (D) AAV-T GM1, and (E) AAV-TC GM1 mice. Group sizes are indicated on the graphs. (C-E) Gray lines show the results for each mouse in the group. Black lines represent the group average.

FIG. 9. βgal Activity in the Feline GM1 Brain Following Intrathalamic Delivery of an AAV-βgal Expression Construct.

A GM1 cat was injected in the right thalamus with 1.85×10¹² g.e. of AAV2/rh8-CBA-fBgal-WPRE, in which a CBA promoter drives expression of a feline βgal cDNA. One month later, the brain was harvested, cryosectioned at 40 μm, and stained with the histochemical substrate Xgal (pH 4.7) to detect lysosomal βgal. To facilitate cryosectioning, large coronal blocks were halved prior to freezing, shown as midline horizontal separations of some sections. βgal was detected throughout the entire injected cerebrum, 1.8 cm anterior and 0.6 cm posterior to the injection (Inj) site. βgal activity ranged from 1.3-4.1 times normal (fold normal (βgal) when quantified with the fluorogenic substrate 4-methylumbelliferyl (4MU)-B-D-galactopyranoside. Xgal-stained control sections are shown from normal and GM1 brain, which expresses <5% normal βgal activity.

FIG. 10. βgal Activity in the Feline GM1 Spinal Cord Following Intracerebroventricular (ICV) Delivery of an AAV-βgal Expression Construct.

Using ultrasound guidance, a GM1 cat was injected into the left lateral ventricle with 4.2×10¹² g.e. of vector AAV2/rh8-CBA-fBgal-WPRE. One month later, the spinal cord was harvested, cryosectioned at 50 μm, and stained for βgal activity with Xgal (pH 5.1). All spinal cord segments in the treated GM1 cat (GM1+AAV) exhibited normal or above normal levels of staining Spinal cord segments are as follows: rostral cervical (RC), mid-cervical (MC), cervical intumescence (CI), mid-thoracic (MT), thoracolumbar (TL), mid-lumbar (ML) and lumbar intumescence (LI). Untreated normal and GM1 spinal cord segments (both LI) are included as controls.

FIG. 11. Quantification of βgal Activity in the Feline GM1 Brain 1 Month Post AAV Treatment.

A GM1 cat was injected bilaterally into the thalamus and deep cerebellar nuclei with AAV2/rh8-CBA-fBgal-WPRE. Brain was sectioned into 0.5 cm coronal blocks for cryoembedding, and blocks were sectioned at 50 um for homogenization and fluorogenic measurement of βgal activity. Distances in cm anterior (+) or posterior (−) to the injection site (Inj) are shown for cerebrum or cerebellum (Cblm). All data was normalized to blocks from normal cats. Untreated GM1 cats expressed <5% normal activity in all blocks.

FIG. 12. Separation of β-Hexosaminidase Isoforms by Ion Exchange Chromatography (Panels A, B) and Western Blotting.

Mouse cerebrum from wild type (WTBrain), untreated Sandhoff mutant (SHBrain), Tay-Sachs mutant (TSBrain), and AAV2/1 α+β co-transduced Sandhoff brain (SHBrain (2/1 α+β; 1:1)) was homogenized and equal amounts of protein loaded into a Resource Q column. Fractions 1-23 were collected and analyzed for hexosaminidase activity using the substrates 4-MUG (A), which detects all three isozymes, and 4-MUGS (B) specific for Hex A and S. The fractionation of the isozymes demonstrates the absence of HexA and HexB in the untreated SD mouse brain; the presence of˜normal amounts of HexB and absence of HexA in the TSD mouse and the abundance of all three isoforms in the WT. In the co-transduced SD brain all three hexosamidases are highly expressed. Assignment of each of the hexosaminidases to the peaks in panel A was corroborated by western blotting (panel C) using an antibody against human Hex A. The unfractionated cerebrum lysate from AAV2/1 α+β co-transduced SD mouse establishes the presence of mature alpha (αm) and beta (βm″a″ and βm″c″) subunits. After fractionation by ion exchange chromatography fraction number 2 contains mainly the beta subunit (Hex B), fraction number 13˜equal amounts of the alpha and the beta subunits (Hex A), and fraction number 17 only the alpha subunit (Hex S).

FIG. 13. Hex Expression and Microglia Immunoreactivity in Two Year-Old AAV-Treated SD mouse brain and spinal cord.

Two year-old AAV-treated SD mice (a-f, j, k); untreated SD mice killed at four months of age (g, l, m); heterozygous littermates (h, I, n, o). β-Hexosaminidase expression in the CNS was assessed by histochemical staining (a-i). Microglial immunoreactivity in the brain (j, l, n) and spinal cord (k, m, o) was assessed with CD68 antibody.

FIG. 14. Intrathalamic Delivery of AAV Vector Formulation in SD Mice.

One month following bilateral intrathalamic injections in 6-8 week-old SD mice, the brains were analyzed for (A) HexA distribution by histochemical staining, and (B) GM2-ganglioside content. Shown is the average+1 SD (n=3). *p<0.01.

FIG. 15. Quantification and Visualization of Glycosphingolipids Stored in Mouse Brain.

Glycosphinglolipids (GSLs) in untransduced and transduced SD mouse brain were analyzed by high-performance thin-layer chromatography (A and B) and electron microscopy (C). (A) GSLs were extracted from a wild type aged 21 weeks (lanes 1, 2, and 13), an untransduced SD mouse aged 16 weeks (lanes 3, 4 and 14), or the brains of Sandhoff mice transduced with rAAVα+β at a single site in the right striatum (lanes 5-12 and 14-18). Vector was injected at 4 weeks of age; the animals were killed at 16 (lanes 5, 6, and 15), 20 (lanes 7, 8, and 16), 24 (lanes 9, 10, and 17), and 30 weeks of age (lanes 11, 12, and 18). Right (lanes 1, 3, 5, 7, 9, and 11) and left cerebrum (lanes 2, 4, 6, 8, 10, and 12) and cerebella (lanes 13-18) were dissected and individually analyzed. Pure GM1, GM2, and GA2 gangliosides and the myelin component, galactocerebroside (Galc), were used as standards (STD). (B) GA2 and GM2 content was quantified densitometrically and is represented as the percentage of the content in the untreated SD mouse, after correcting for loading differences, by using the internal Galc standard. Storage was diminished in all treated SD brains but increased progressively with age. (C) Neuronal ultrastructure in brain sections from wild-type (d), untransduced (c), and singly rAAVα+β-transduced SD mice (a and b). A single striatal injection of viral vector was given at 4 weeks, and the tissue harvested at 16 weeks of age. Neurons in the transduced ipsilateral cerebral cortex had no membranous cytoplasmic cell bodies (b), whereas those in the contralateral cortex (a) were distended by the storage vesicles (arrowheads in a) with distortion of the nuclei, as in untreated SD animals (c). N, nucleus. (Scale bar: 2 μm.)

FIG. 16. Relationship Between β-Hexosaminidase Activity, Glycosphingolipid Storage, and Inflammatory Cells in the Cerebral Cortex.

Coronal sections from wild type aged 16 weeks (a-d), rAAV2/2α+β-transduced aged 29 weeks (humane end point) (e-h), and untransduced SD mice aged 17 weeks (i-l) were prepared consecutively. Virus was injected at 4 weeks of age. The β-hexosaminidase reaction product stains red (a and e) and is absent in untransduced Sandhoff mice (i). Glycospingolipid storage, detected by neuronal PAS staining, occurs particularly in layers IV and V of the cerebral cortex of untreated SD mice (arrowheads in 1) but was undetectable in cortex from wild-type (d) or transduced SD mice (h). Activated microglia/macrophages were recognized by immunostaining of the cell-specific marker, CD68 (b, f, and j), and by binding to isolectin B4 (c, g, and k). No cells of microglia/macrophage lineage were detected in wild-type cortex (b and c), and only a few were seen in transduced Sandhoff mice (arrowheads in f and g). Cerebral cortex from untransduced Sandhoff mice contained numerous activated microglia and macrophages (arrowheads in j and k). The number of neurons staining with PAS and the presence of cells recognized by G. simplicifolia isolectin B4 (GSIB4) and CD68 antibodies inversely depended on enzymatic activity.

FIG. 17. Survival, Weight, and Neurological Function after Gene Therapy in SD Mice.

Weight and rescue of neurological function was assessed in wild-type, untransduced, and transduced SD mice. Transduced animals were injected at 4 weeks of age. (A) Range of body weights in wild-type mice [light grey and dark grey stippled area for males (n=6) and females (n=7), respectively], untransduced (shaded triangles; n=1) and rAAVα-transduced (dark squares; n=1) SD males, untransduced (shaded squares; n=5) SD females, and rAAV2/2β or rAAV2/1β-transduced SD males at four sites (open triangles; n=3). After therapy, SD mice gained and maintained their weight normally. (B) Effect of therapy on hind-limb movements in WT, untransduced or rAAVα-transduced SD animals, and SD mice after transduction with either rAAV2/2β, rAAV2/1β, or rAAV2/2α+β (Treated SD). Each dot represents a single animal. Over 120 days, movement frequency declined in SD mice (P=0.0108) but, in treated SD animals, remained indistinguishable from WT (P=0.1107); limb movements improved significantly after gene therapy (P<0.0001).

FIG. 18. Effects of Gene Therapy on Survival of SD Mice.

Animals treated by gene therapy were given either a single injection of AAV coding for human beta-hexosaminidase in the right striatum or four injections (bilaterally in the striatum and cerebellum) at four weeks of age. Untreated SD mice reached their pre-defined humane endpoint at around 120 days of age, those treated at a single site at 200 days, but about 25% of the animals given four injections, at this particular vector dose, were still alive at one year of age.

FIG. 19. AAV-Treated SD Mice Display Improved Performance in the Inverted Screen Test Compared to Untreated SD Mice.

(A) Performance of AAV2/1-treated mice compared to untreated SD (MT) and heterozygote (WT) control mice. For AAV2/1-treated mice, Hex subunits were tested with (α1, β1) or without (α4, β4) a carboxyl-terminal fusion of the HIV Tat protein transduction domain. (B) Performance of AAV2/rh8-treated mice compared to untreated SD (MT) and heterozygote (WT) control mice. Two comparisons were performed between AAV2/rh8-treated SD mice (123 and 246 days of age) and untreated SD mice (123 days of age) using the Mann-Whitney test. A significant difference in performance in the Inverted Screen Test at P<0.05 was found when comparing the two groups at 123 days of age.

FIG. 20. AAV-Treated SD Mice Sustain Performance in Accelerating Rotarod Test Over Time.

(A) Performance of AAV2/1-treated SD mice compared to untreated SD (MT) and heterozygote (WT) control mice. For AAV2/1-treated mice, Hex subunits were tested with (α1, β1) or without (α4, β4) a carboxyl-terminal fusion of the HIV Tat protein transduction domain. (B) Performance of AAV2/rh8-treated mice compared to untreated SD (MT) and heterozygote (WT) control mice. Two comparisons were performed between AAV2/rh8-treated SD mice (123 and 246 days of age) and untreated SD mice (123 days of age) using the Mann-Whitney test. No significant differences were found in performance in the Accelerating Rotarod test at P<0.05 between the two groups.

FIG. 21. Sustained Performance of AAV-Treated SD Mice in Barnes Maze Test.

(A) Performance of AAV2/1-treated SD mice compared to untreated SD (MT) and heterozygote (WT) control mice. For AAV2/1-treated mice, Hex subunits were tested with (α1, β1) or without (α4, β4) a carboxyl-terminal fusion of the HIV Tat protein transduction domain. (B) Performance of AAV2/rh8-treated mice compared to untreated SD (MT) and heterozygote (WT) control mice. Two comparisons were performed between AAV2/rh8-treated SD mice (123 and 246 days of age) and untreated SD mice (123 days of age) using the Mann-Whitney test. The performance of AAV2/rh8-treated SD mice was significantly (P<0.05) better than untreated SD mice at either age analyzed.

FIG. 22 β-Hexosaminidase Enzymatic Activity in the Cerebrum and Cerebellum of Heterozygous (Hexb+/−), SD (Hexb−/−), and AAV-Treated SD Mice.

A) Right cerebrum coronal sections (R1-R4). B) Right cerebellum. Activities were measured in frozen tissue sections using 4-methyumbelliferyl-N-acetyl-β-D-glucosaminide as the substrate. Values are expressed as the mean±SEM. N=3, 4, and 6 mice per group for heterozygote (HZ), untreated SD (KO), and AAV-treated SD (KO) mice, respectively. Asterisks denote statistical significance with a p-value <0.05 using a student's two-tailed t-test.

FIG. 23. AAV-Mediated β-Hexosaminidase Expression Reduces Total Ganglioside Content and Corrects GM2 Storage in SD Mouse Cerebrum.

A) HPTLC of cerebrum gangliosides. Approximately 1.5 μg of sialic acid was spotted per lane from pooled right cerebrum sections. B) Total sialic acid content quantified using the resorcinol assay. C) GM2 content quantified via densitometric scanning of the HPTLC plate in A. Values are expressed as mean±SEM. N=3, 4, and 6 mice per group for HZ, untreated SD (KO), and AAV-treated SD (AAV) mice, respectively. Asterisks denote a statistically significant difference (p<0.001) from the untreated SD (KO) mice using one-way ANOVA.

FIG. 24. AAV-Mediated (1-Hexosaminidase Expression Reduces Total Ganglioside Content and Corrects GM2 Storage in SD Mouse Cerebellum.

A) HPTLC of cerebellar gangliosides. Approximately 1.5 μg of sialic acid was spotted per lane from right cerebellum sections. B) Total sialic acid content quantified using the resorcinol assay. C) GM2 content quantified via densitometric scanning of the HPTLC plate in A. Values are expressed as mean±SEM. N=3, 4, and 6 mice per group for HZ, untreated SD (KO), and AAV-treated SD (AAV) mice, respectively. Asterisks denote a statistically significant difference (p<0.001) from the untreated SD (KO) mice using one-way ANOVA.

FIG. 25. Influence of AAV Gene Therapy on Myelin-Associated Cerebrosides and Sulfatides in SD Mouse Brain.

Neutral and Acidic lipids purified from A) right cortex and B) right cerebellum were spotted on HPTLC at 70 ug and 200 ug/mg dry tissue weight, respectively. Values for cerebrosides and sulfatides were taken from densitometric scanning of HPTLC plates (data not shown). Values are expressed as mean±SEM. N=3, 4, and 6 mice per group for HZ, untreated SD (KO), and AAV-treated SD (AAV) mice, respectively. Asterisks denote a statistically significant difference (p<0.05 and p<0.01, respectively) from the untreated SD (KO) mice using one-way ANOVA.

FIG. 26. Effect of AAV-Treatment on Disease Marker Gene Expression in the CNS of SD Mice.

Expression levels of disease marker genes in AAV2/rh8-treated SD mice at 8 months of age (red bars), and untreated SD mice at humane endpoint (black bars) normalized for levels in 8 month-old untreated heterozygote animals. Show is the mean±SEM. N=3 for each structure analyzed. Dotted line indicates normal expression levels. Asterisks denote statistical significance with a p-value <0.05 using a student's one-tailed t-test.

FIG. 27. Distribution of β-Hexosaminidase Activity in the Brain of GM2 Cats 16 Weeks Post-Injection of AAV2/rh8 Vectors.

(A) Histochemical staining for hexosaminidase activity was used to analyze enzyme distribution throughout the brain of AAV-treated GM2 cats (GM2+AAV). Stained sections from untreated normal and untreated GM2 cats are shown for comparison. Enzymatic assays of the same brain regions were performed for (B) hexosaminidase (HexA and total Hex using MUGS or MUG substrates, respectively) and (C) lysosomal acid beta-galactosidase, and were expressed as fold-over normal.

FIG. 28. Neurochemical Analysis of Different CNS Regions in AAV-Treated GM2 Cats at 16 Weeks Post-Injection.

(A) The brain was divided into coronal blocks and then subdivided into quadrants. The quadrants circled in red and also the striatum and thalamus were isolated to analyze the neurochemistry in AAV-treated GM2 cats and controls. Analysis has been concluded for regions 3, 7, 20, 22, striatum and thalamus. (B) Total sialic acid content (μg/100 mg dry weight); (C) GM2-ganglioside content (μg/100 mg dry weight; absent in normal CNS); (D) Cerebroside content (μg/mg dry weight). Grey bars represent the mean values for AAV-treated GM2 cats (N=3). Error bars=1 standard deviation. Values in normal cat brain (N=1, green circles), mean values in untreated GM2 cats (N=2, red crosses). Tables below each graph show the means values. Abbreviations: Ctx—cerebral cortex; Cb—Cerebellum; Str—Striatum; Tha—Thalamus.

FIG. 29. Ganglioside Distribution in Different Brain Structures of AAV-Treated GM2 Cats.

High-performance thin layer chromatography plates of gangliosides in different regions of the CNS of AAV-treated GM2 cats and controls is shown. GM2-ganglioside content was calculated by densitometric scanning of the plates. Abbreviations: NM—Normal control; SD—untreated GM2 cat.

FIG. 30. Neurochemical Analysis of Spinal Cord in AAV-Treated Sandhoff Cats at 16 Weeks Post-Injection.

(B) Total Hexosaminidase activity (C) Total sialic acid content (μg/100 mg dry weight); (D) GM2-ganglioside content (μg/100 mg dry weight; absent in normal spinal cord); (E) GA2 content (μg/100 mg dry weight). No GA2 was detectable in lumbar spinal cord in any of the AAV-treated SD cats. Grey bars represent the mean values for AAV-treated SD cats (N=3). Error bars=1 standard deviation. Values in normal cat brain (N=1, green circles), mean values in untreated SD cats (N=2, red circles). Abbreviations: SC—Spinal cord.

FIG. 31. Growth Rates of Normal, Untreated GM2 and AAV-Treated GM2 Cats.

Cats in the study were weighed at least every 2 weeks, and plots of weight versus age were constructed. To the data points were added a best fit linear trend line (Microsoft Excel), and the slope of the trend line was calculated to determine the growth rate. Because growth rates decrease with age, rates were calculated for 2 separate age ranges: 5-18 weeks and 5-24 weeks. The left-hand panel depicts growth rates in kg/week, while the right-hand panel provides an example of typical growth curves (Normal, 7-735; GM2+AAV, 11-732; GM2, 7-682).

FIG. 32. Magnetic Resonance Images of Normal, AAV-Treated GM2 and Untreated GM2 Cats at 5 Months of Age.

As shown in T2 and T1 weighted images, the AAV-treated GM2 cat (GM2+AAV, 7-714) demonstrated remarkably fewer indicators of brain deterioration than the untreated GM2 cat. Noticeably milder brain abnormalities in the treated GM2 cat were documented in gyms width, sulcus depth and width, ventricular width, and white-gray matter signal relationships in corona radiata and internal capsule (not shown). Bilaterally decreased signal in the geniculate bodies was a consistent finding in both untreated and AAV-treated cats. Normal, 7-681; GM2+AAV, 7-714; GM2 untreated, 7-681. See text for further details.

FIG. 33. Gait Analysis of Normal and AAV-Treated GM2 Cats.

Cats walked without external manipulation (leashes, etc.) from sensor initiation to sensor termination points (from right to left in the diagram as indicated by paw direction on the gray inset). (A) Coded paw identification is as follows: right fore (RF), right hind (RH), left fore (LF), left hind (LH). The AAV-treated cat (7-714) demonstrated mild, quantifiable gait abnormalities at 5.6 months of age, >1 month past the humane endpoint for untreated GM2 cats. As shown in the gait panels, 7-714 exhibits a shorter than normal stride length and shorter than normal reach, especially on the left side (note overlap of blue and green sensor images). [Reach is defined as the distance from heel center of the hind paw to heel center of the previous fore paw.] (B) Several quantitative measures of gait were recorded from sensor activation during ambulation and are presented. Note the deviation from normal in cat 7-714 for Left Reach (10.6), maximum pressure ratio (fore-hind, 0.93) and left-right symmetry (1.12). This data suggests stronger gait abnormalities on the left side, a difference not readily apparent by observation or neurologic examination. Maximum pressure ratios define the amount of pressure that the animal places on fore paws versus hind paws, and the symmetry ratio describes the equality of pressure between left and right sides.

### DETAILED DESCRIPTION

The present disclosure relates generally to methods for the treatment of lysosomal storage disorders with AAV-mediated gene therapy. In particular, the present disclosure provides methods for treating, reducing the severity of, or delaying the onset of Tay-Sachs Disease and Sandhoff Disease by providing AAV-mediated HexA expression in the brain of a subject in need thereof. AAV-mediated HexA expression is achieved by administering pharmaceutical compositions comprising AAV-HexA expression constructs to the brain of the subject.

In practicing the present disclosure, many conventional techniques in cell biology, molecular biology, protein biochemistry, immunology, and bacteriology are used. These techniques are well-known in the art and are provided in any number of available publications, including Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989).

The definitions of certain terms as used in this specification are provided below. Unless defined otherwise, all technical and scientific terms used herein generally have the same meaning as commonly understood by one of ordinary skill in the art to which this invention belongs.

As used herein, the singular forms “a,” “an,” and “the” include plural referents unless the content clearly indicates otherwise. For example, reference to “a cell” includes a combination of two or more cells, etc.

As used herein, “AAVrh.8 vector” refers to AAV an AAV vector carrying the Adeno-associated virus isolate AAVrh.8 capsid protein (VP1) gene. An exemplary sequence for AAVrh.8 is given by GenBank Accession No. AY242997. As used herein, the term encompasses natural and engineered AAVrh.8 variants. In some embodiments, variants have about 60% identity, and in some embodiments 65%, 70%, 75%, 80%, 85%, 90%, 91%, 92%, 93%, 94%, 95%, 96%, 97%, 98%, 99%, or higher identity over a specified region.

As used herein, the “administration” of an agent, drug, or peptide to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another. In some embodiments, “administration” refers to direct infusion of pharmaceutical compositions comprising AAV expression constructs into the brain parenchyma. Compositions may be administered at any site within the brain sufficient to result in widespread distribution and expression of the constructs. In some embodiments, the infusion site is chosen from the group consisting of the thalamus, striatum, deep cerebellar nuclei, ventral tegmental area, and lateral ventricles.

As used herein, “β-N-acetylhexosaminidase,” “β-Hexosaminidase,” and “HexA” refer to the mammalian enzyme composed of α and β subunits encoded by the HEXA and HEXB genes, respectively. The term encompasses full-length molecules, variants, isoforms, and fragments that retain enzymatic activity against HexA substrates. Exemplary nucleic acid and sequences are given by GenBank Accession Nos. HexA: NM_{—}000520; BC018927; HexB: NM_{—}000521. The term encompasses natural and engineered molecules identical or substantially identical to these sequences. The term “HexA expression constructs” or “HexA expression vectors” refers constructs encoding both the α and β HexA subunits, as in the context of a pharmaceutical composition.

As used herein, “acid β-galactosidase,” “β-galactosidase,” and “βgal” refer to the mammalian enzyme encoded by the exemplary sequences given by GenBank Accession No. NM_{—}000404. The term encompasses full-length molecules, variants, isoforms, and fragments that retain enzymatic activity against βgal substrates. The term encompasses natural and engineered molecules identical or substantially identical to this exemplary sequence.

As used herein, the term “effective amount” or “pharmaceutically effective amount” or “therapeutically effective amount” or “prophylactically effective amount” of a composition, is a quantity sufficient to achieve or maintain a desired therapeutic and/or prophylactic effect, e.g., an amount which results in the prevention of, or a decrease in, the symptoms associated with a disease that is being treated, e.g., a cancer. The amount of a composition of the invention administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. In the context of treating a lysosomal storage disorder, in some embodiments, an effective amount is the amount sufficient to cause a decrease in the severity of symptoms associated with the disorder. In the context of prophylactic administrations, in some embodiments, an effective amount is the amount sufficient to delay the onset of or decrease the likelihood of onset of a lysosomal storage disorder.

As used herein, the terms “isolate” and “purify” refer to processes of obtaining a biological substance that is substantially free of material and/or contaminants normally found in its natural environment (e.g., from the cells or tissues from which a protein is derived, or substantially free from chemical precursors or other chemicals when chemically synthesized).

As used herein, “expression” includes, but is not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the terms “identical,” substantial identity,” or percent “identity”, when used in the context of two or more nucleic acids or polypeptide sequences, refers to two or more sequences or subsequences that are the same or have a specified percentage of amino acid residues or nucleotides that are the same, e.g., about 60% identity, in some embodiments 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site). Such sequences are then said to be “substantially identical.” This term also refers to, or can be applied to, the complement of a test sequence. The term also includes sequences that have deletions and/or additions, as well as those that have substitutions. As described below, the preferred algorithms can account for gaps and the like. In some embodiments, identity exists over a region that is at least about 25 amino acids or nucleotides in length. In other embodiments, identity exists over a region that is 50-100 amino acids or nucleotides in length.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons. The term encompasses any of the known base analogues of DNA and RNA such as, but not limited to 4-acetylcytosine, 8-hydroxy-N6-methyladenosine, aziridinyl cytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosyl queosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term DNA “control sequences” includes but is not limited to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

As used herein, the term “coding sequence” refers to a nucleic acid which is transcribed and/or translated into a polypeptide in vitro or in vivo when placed under the control of appropriate regulatory sequences. The term is used interchangeably with references to sequences that “encode” a particular protein or polypeptide. The boundaries of the coding sequence are determined by a start codon at the 5′ (amino) terminus and a translation stop codon at the 3′ (carboxy) terminus A coding sequence can include, but is not limited to, cDNA derived from prokaryotic or eukaryotic mRNA, prokaryotic or eukaryotic genomic DNA, and synthetic DNA sequences. A transcription termination sequence will usually be located 3′ to the coding sequence

As used herein, the term the terms “polypeptide,” “peptide,” and “protein” are used interchangeable to mean a polymer comprising two or more amino acids joined to each other by peptide bonds or modified peptide bonds (i.e., peptide isosteres). Polypeptides may include amino acids other than the naturally-occurring amino acids, as well as amino acid analogs and mimetics prepared by techniques that are well known in the art. The skilled artisan will understand that polypeptides, peptides, and proteins may be obtained in a variety of ways including isolation from cells and tissues expressing the protein endogenously, isolation from cell or tissues expressing a recombinant form of the molecule, or synthesized chemically.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, e.g., recombinant cells express genes that are not found within the native (non-recombinant) form of the cell or express native genes that are otherwise abnormally expressed, under expressed or not expressed at all.

As used herein, the term “subject” refers to an organism administered one or more compositions of the invention. Typically, the subject is a mammal, such as an animal, e.g., domestic animals (e.g., dogs, cats and the like), farm animals (e.g., cows, sheep, pigs, horses and the like) and laboratory animals (e.g., monkey, rats, mice, rabbits, guinea pigs and the like). In some embodiments, the subject is a human.

As used herein, the term “substitution” carries the meaning generally understood in the art. Protein variants having at least one amino acid residue exchanged for another are said to have a substitution. “Conservative substitutions” typically result similar physical properties as the unmodified polypeptide sequence from which the variant was derived. Conservative substitutions typically include the substitution of an amino acid for one with similar characteristics. Conservative substitution tables providing functionally similar amino acids are well known in the art. For example, the following six groups each contain amino acids that are conservative substitutions for one another: 1) Aliphatic: glycine (G), alanine (A), valine (V), leucine (L), isoleucine (I); 2) Aromatic: phenylalanine (F), tyrosine (Y), tryptophan (W); 3) Sulfur-containing: methionine (M), cysteine (C); 4) Basic (Cationic): arginine (R), lysine (K), histidine (H); 5) Acidic (Anionic): aspartic acid (D), glutamic acid (E); 6) Amide: asparagine (N), glutamine (Q).

As used herein, terms “transformation,” “transfection” and “transduction” refer to the uptake of foreign nucleic acid by a cell. A cell is said to have been “transformed,” “transfected” or “transduced” when exogenous nucleic acid has been introduced inside the cell membrane. A number of transfection techniques are generally known in the art See, e.g., Graham et al. (1973) Virology, 52 456, Sambrook et al. (1989) Molecular Cloning, a laboratory manual, Cold Spring Harbor Laboratories, New York, Davis et al (1986) Basic Methods in Molecular Biology, Elsevier, and Chu et al. (1981) Gene 13 197. Such techniques can be used to introduce one or more exogenous nucleic moieties, such as a nucleotide integration vector and other nucleic acid molecules, into suitable host cells

As used herein, the term “host cell” refers to, for example, microorganisms, yeast cells, insect cells, and mammalian cells, that can be, or have been, used as recipients of an exogenous nucleic acid. The term encompasses the progeny of the original transfected call. Thus, a “host cell” as used herein generally refers to a cell which has been transfected with an exogenous nucleic acid sequence. It is understood that the progeny of a single parental cell may not necessarily be completely identical in morphology or in genomic or total nucleic acid complement as the original parent, due to natural, sporadic, or deliberate mutation.

As used herein, the terms “treating” or “treatment” or “alleviation” refers to both therapeutic treatment and prophylactic or preventative measures, wherein the object is to prevent or slow down (lessen) the targeted pathologic condition or disorder. A subject is successfully “treated” for a lysosomal storage disorder if after receiving a therapeutic amount of an AAV expression construct according to the methods disclosed herein, the subject shows observable and/or measurable reduction in or absence of one or more signs and symptoms of the disorder, including but not limited to improved processing of gangliosides, improved motor control, visual acuity, and/or increased longevity.

As used herein, the term “predisposed to having” a lysosomal storage disorder refers to subjects with a family history of a lysosomal storage disorder such that there is a possibility that the subject has inherited one or more genetic loci comprising disease loci and will at some point develop a diagnosable disorder. The term also encompasses subject heterozygous or homozygous at a single disease locus or multiple disease loci.

The term “suspected of having” refers to subjects who present with clinical or biochemical symptoms associated with a lysosomal storage disorder, regardless of whether they have been diagnosed as having the disorder.

As used herein, the term “hexosaminidase deficiency” refers to reduced expression or function of hexosaminidase compared to normal levels for sex and age matched subjects. Deficiencies may be the result genetic mutations or other molecular events that impair transcription, translation, post-translational modification, sub-cellular localization, dimerization, or enzymatic function of the hexosaminidase α and β subunits. The severity of hexosaminidase deficiency may vary across subjects, and or may not result in clinical symptoms associated with lysosomal storage disorders.

As used herein, the term “acid β-galactosidase deficiency” refers to reduced expression or function of the enzyme compared to normal levels for sex and age matched subjects. Deficiencies may be the result genetic legions or other molecular events that impair transcription, translation, post-translational modification, sub-cellular localization, or enzymatic function of the enzyme. The severity of acid β-galactosidase deficiency may vary across subjects, and or may not result in clinical symptoms associated with lysosomal storage disorders.

As used herein, the term “unilateral administration” refers to administration of pharmaceutical compositions at loci restricted to one hemisphere of the brain. In the context of unilateral administration, “ipsilateral” refers to the hemisphere to which the composition was administered; “contralateral” refers to the opposite hemisphere. The term “bilateral” refers to administration of pharmaceutical compositions at loci in both hemispheres of the brain. In the context of bilateral administration, administration of compositions may or may not be symmetrical with respect to the brain as a whole. In the context of bilateral administration, the specific loci of administration may or may not be the same for both hemispheres. In some embodiments, pharmaceutical AAV compositions are administered unilaterally. In some embodiments, pharmaceutical AAV compositions are administered bilaterally. In some embodiments, a subject may receive both unilateral and bilateral administrations at different time points.

As used herein, “evaluating” enzyme activity in a subject administered AAV compositions refers to assessing the activity of a replacement enzyme administered via the composition. “Evaluation” of subjects may comprise assessment of symptoms associated with enzyme deficiency, such as symptoms associated with lysosomal a storage disorder. In some embodiments, the disorder comprises Tay-Sachs Disease or Sandhoff Disease. In other embodiments, the disorder comprises GM1-gangliosidosis. As used herein, “evaluating” also encompasses biochemical assessment of enzyme activity, such as biochemical assessment of hexosaminidase activity or β-galactosidase activity. As used herein, the term encompasses evaluation of enzyme activity comprising a combination of clinical and biochemical assessment of enzyme activity.

As used herein, “widespread distribution” refers to exogenous enzyme expression and/or activity in a region of the brain substantially greater than the area immediately surrounding the site of infusion. The experimental data shows presence of active enzyme throughout the entire brain and spinal cord after delivery of AAV vectors to the thalamus and deep cerebellar nuclei of mice and cats (GM1 and GM2 models). Enzyme is found throughout the cerebral cortex and sub-cortical structures (e.g. striatum, thalamus, hyppothalamus, hippocampus, brainstem), and spinal cord.

### Lysosomal Storage Diseases

### GM1 Gangliosidosis

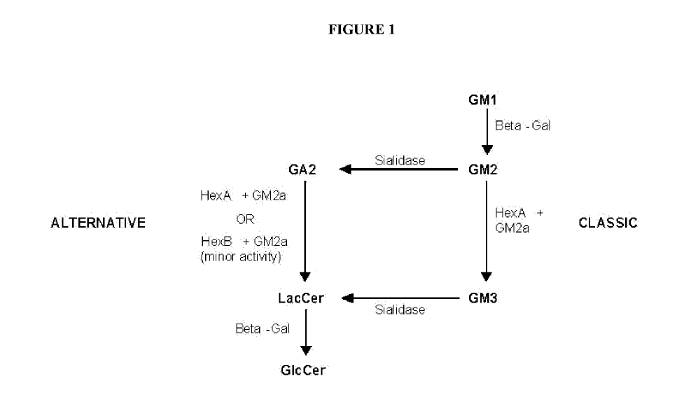

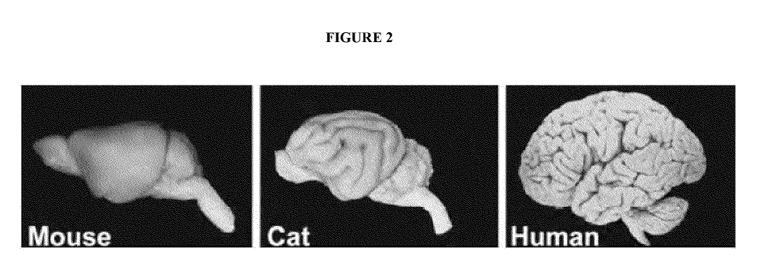

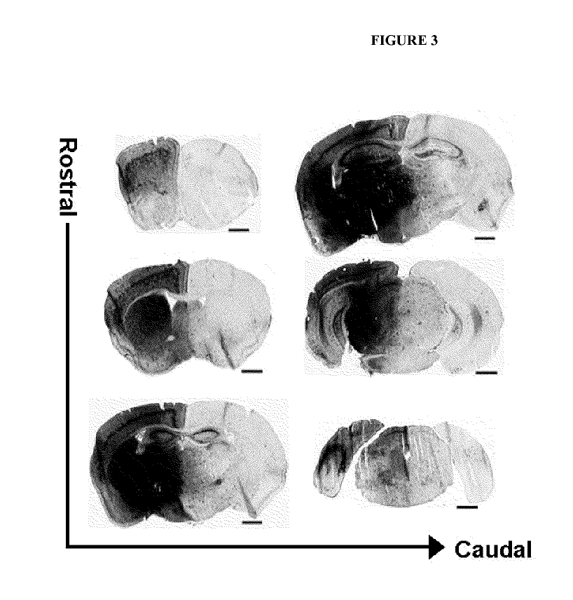

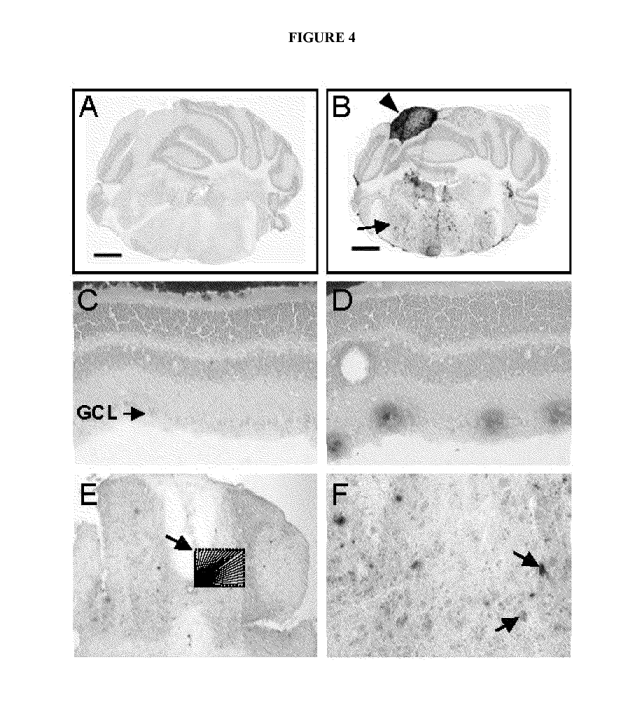

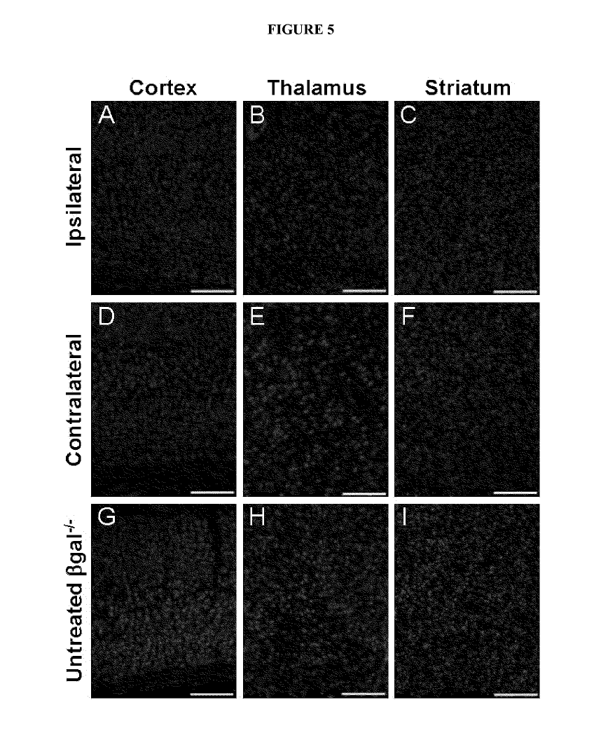

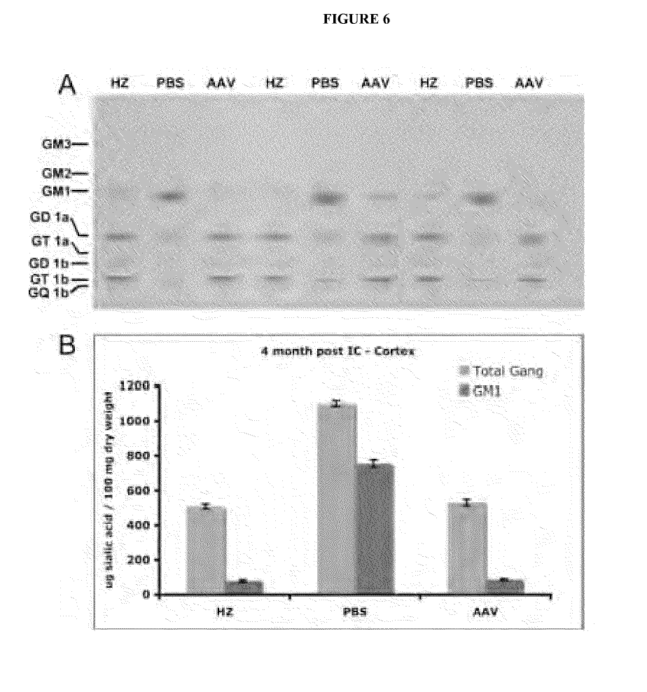

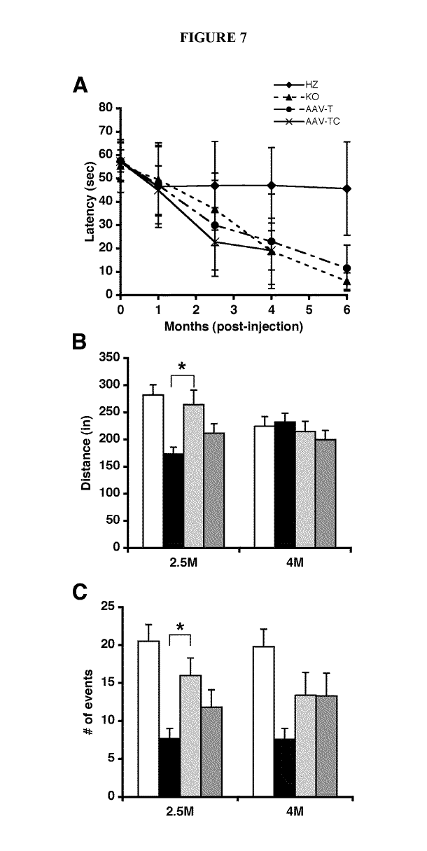

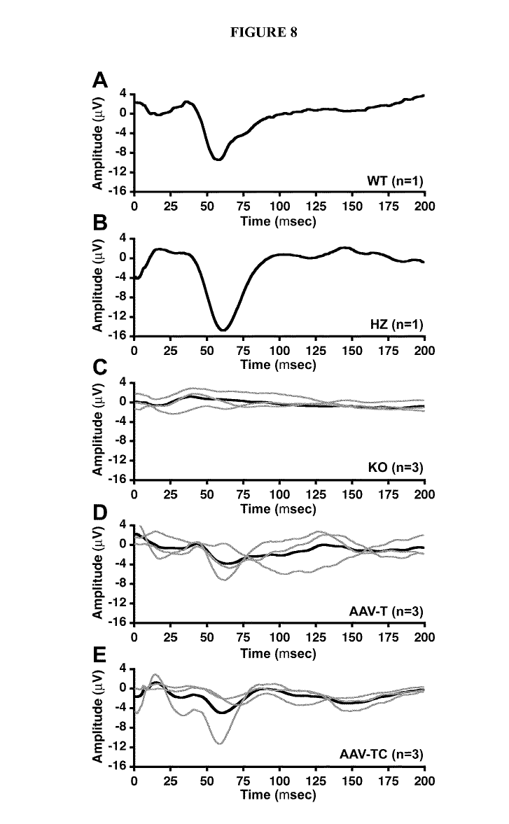

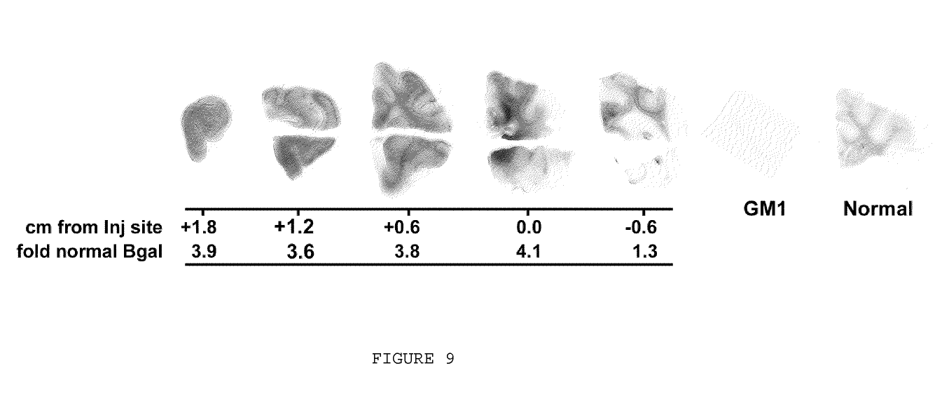

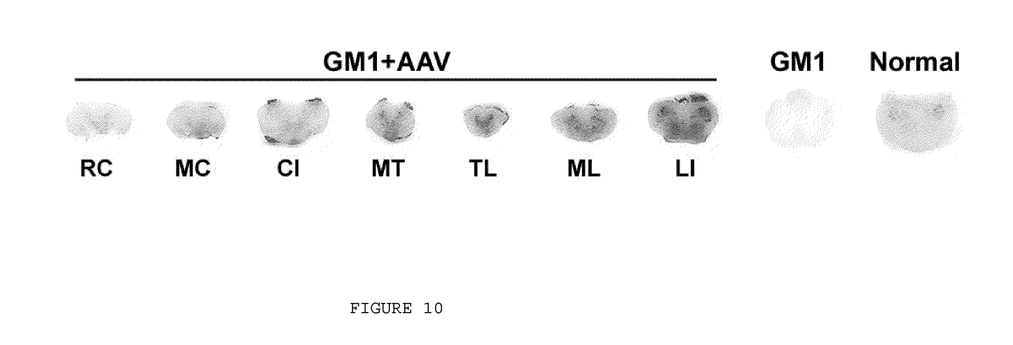

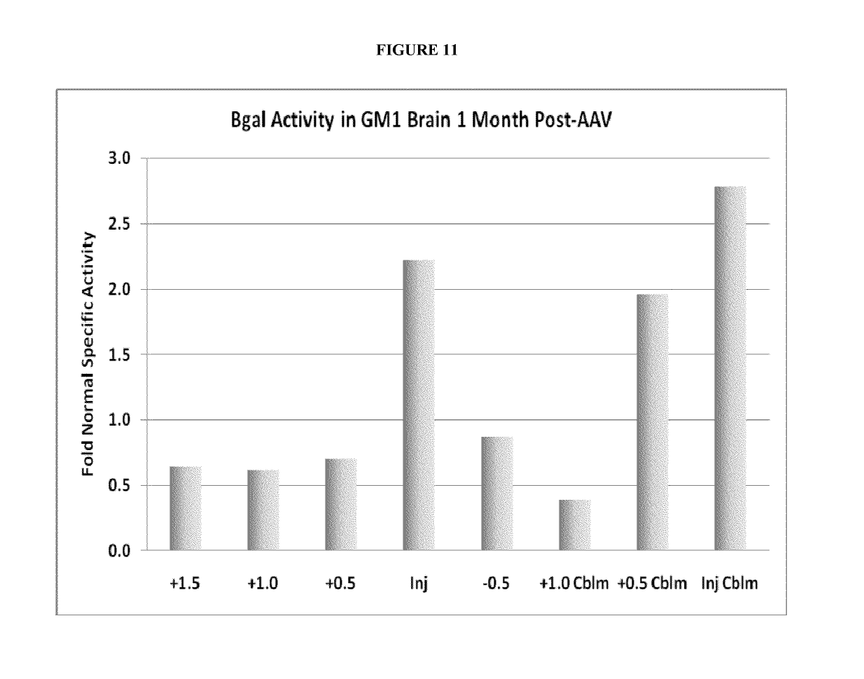

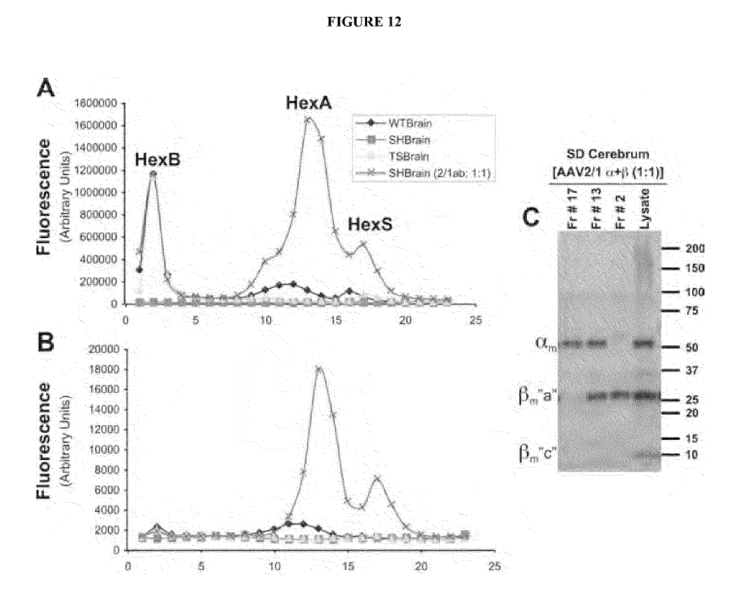

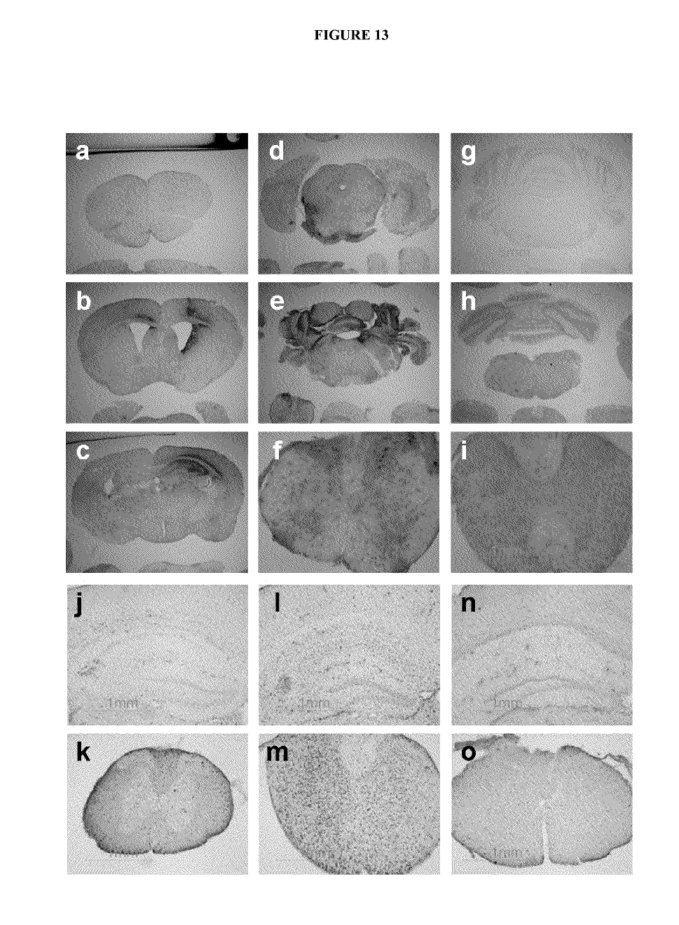

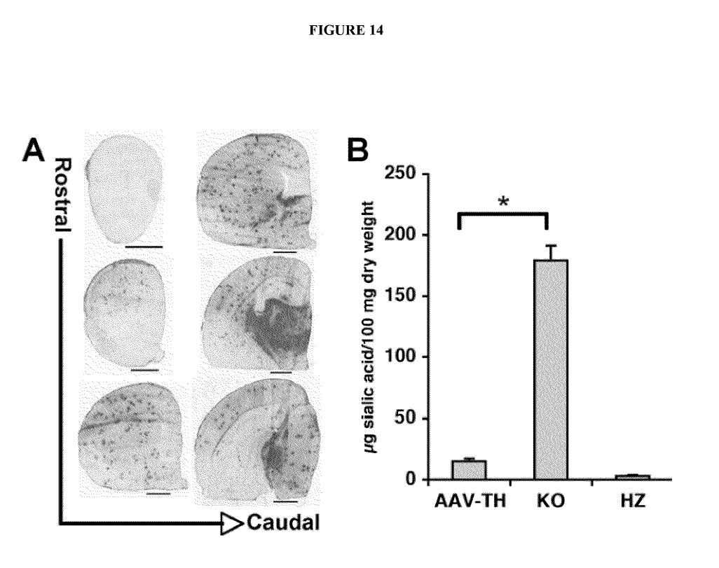

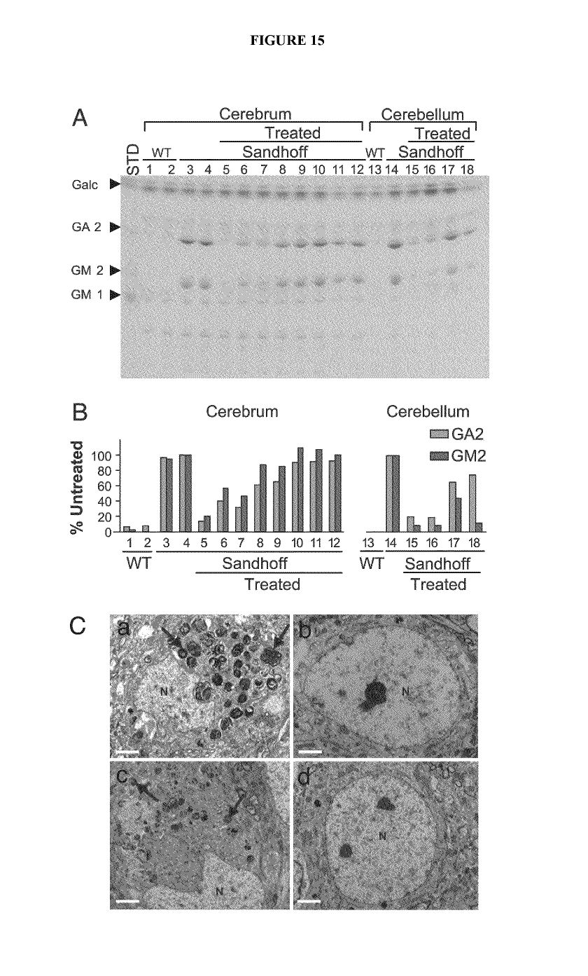

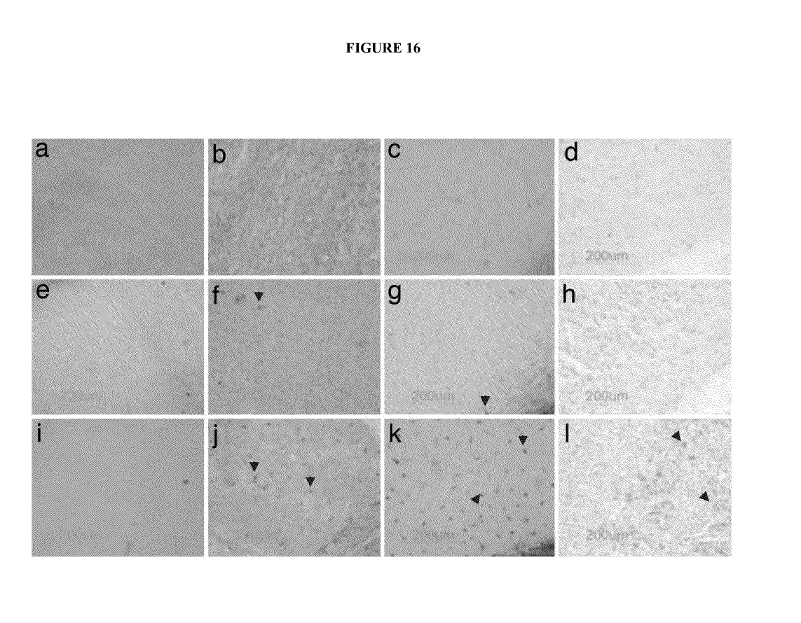

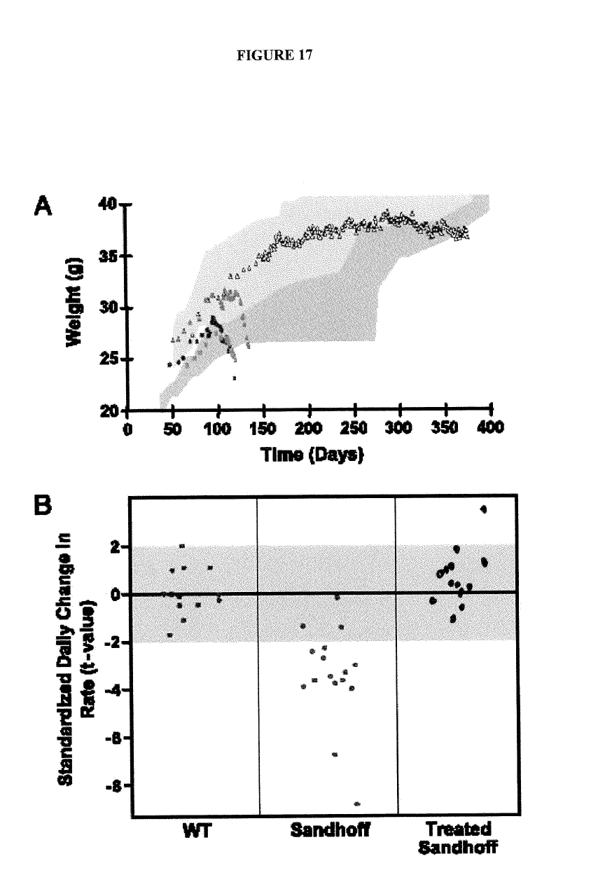

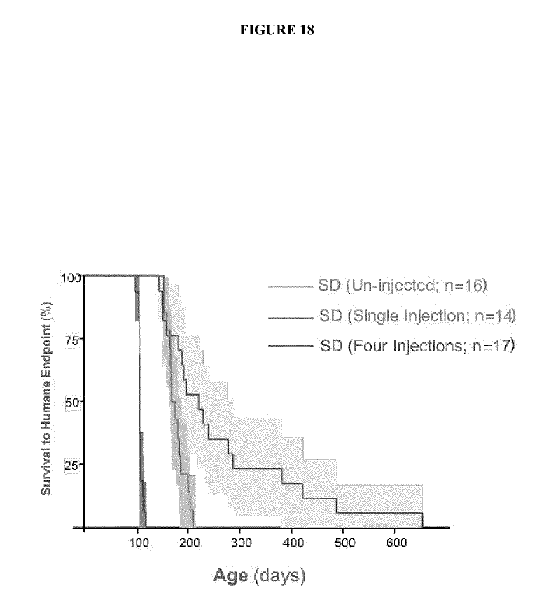

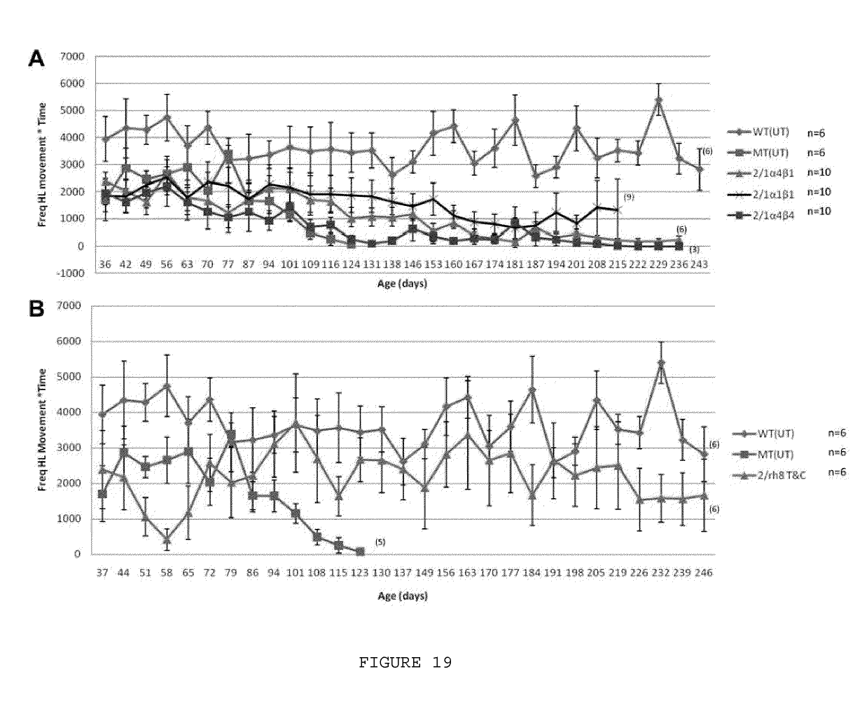

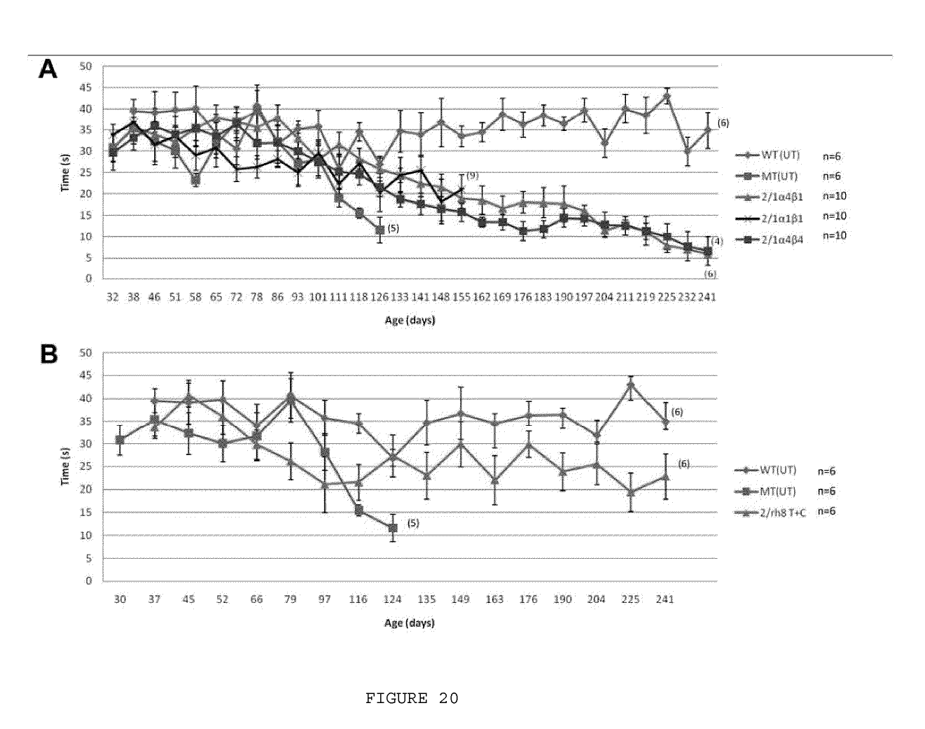

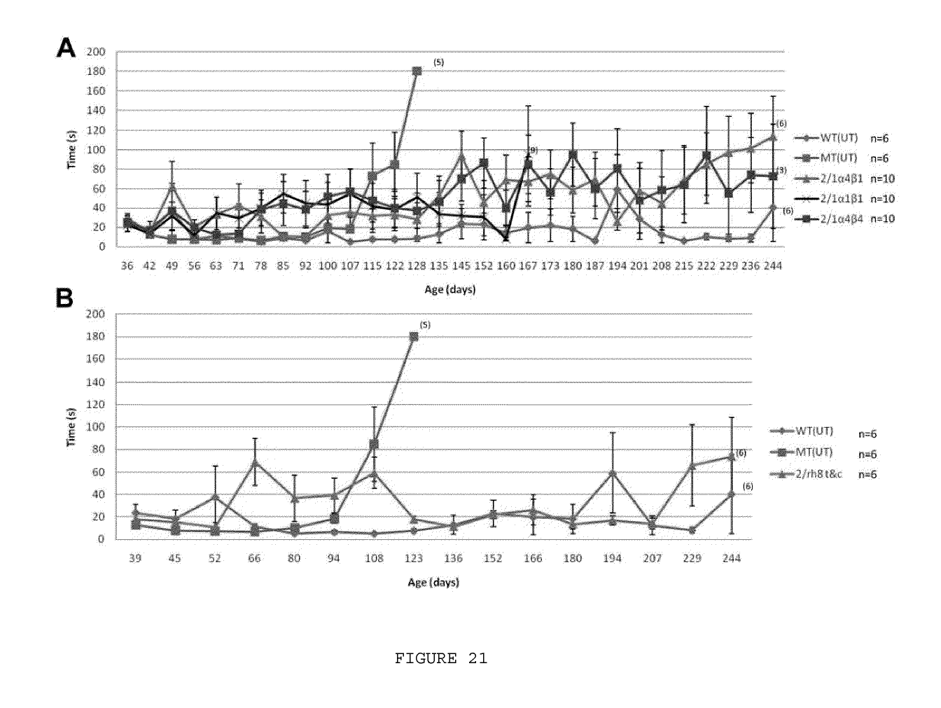

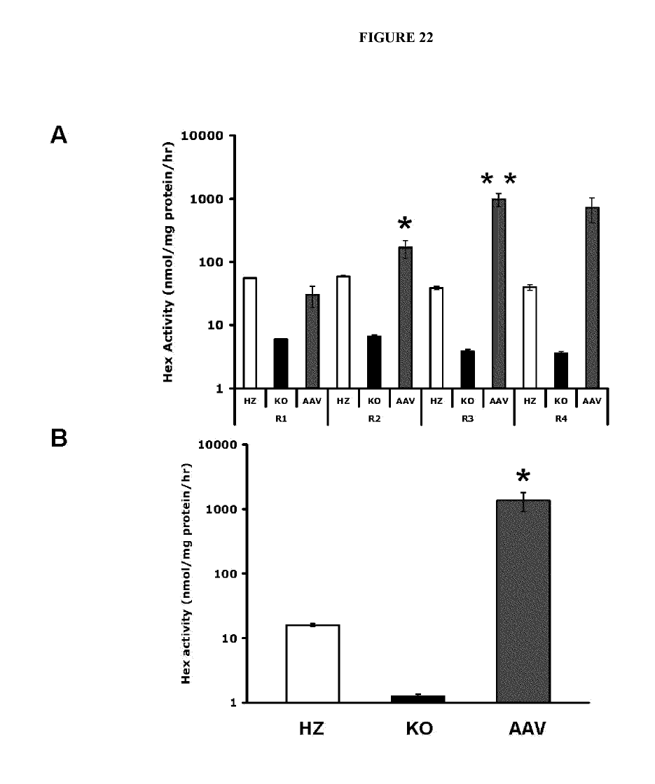

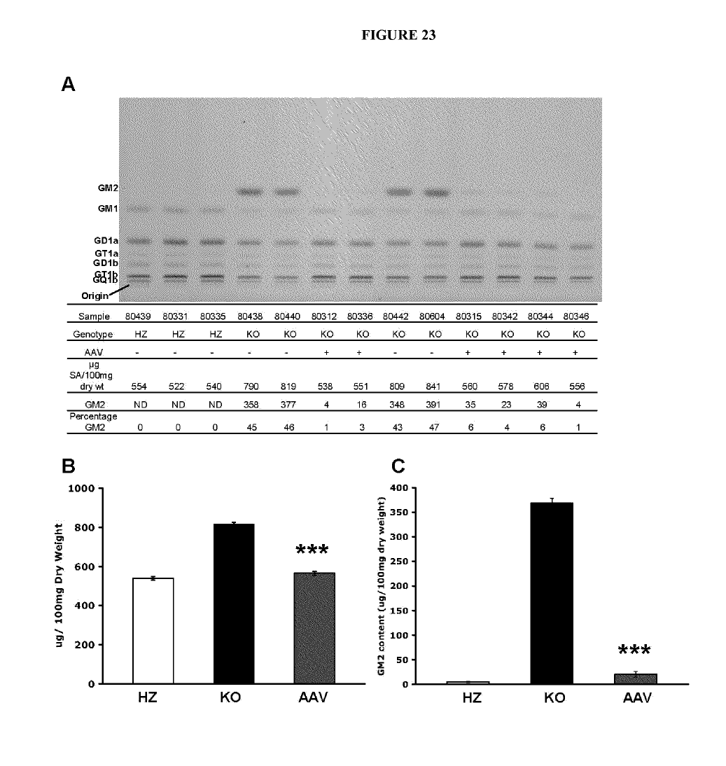

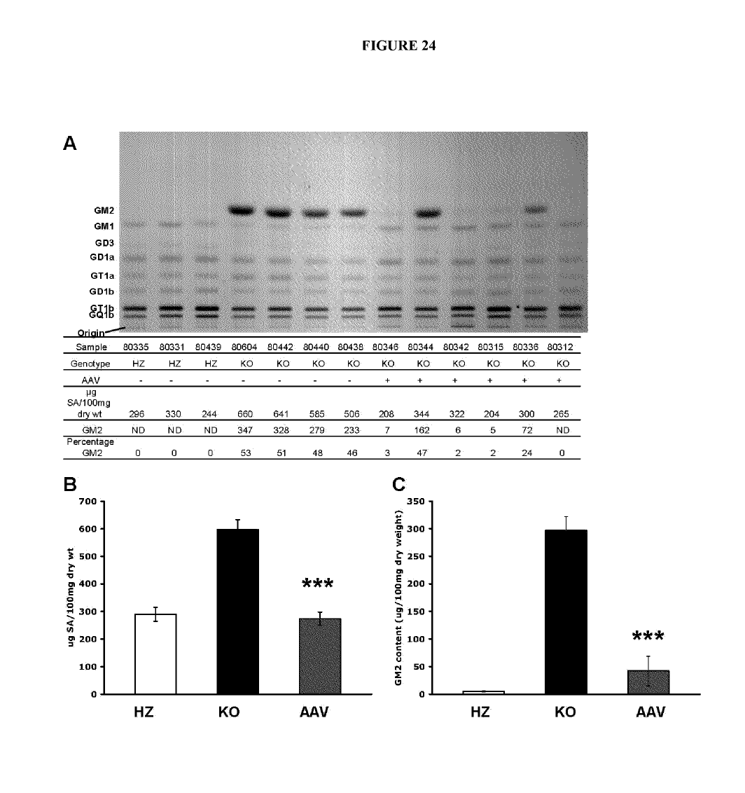

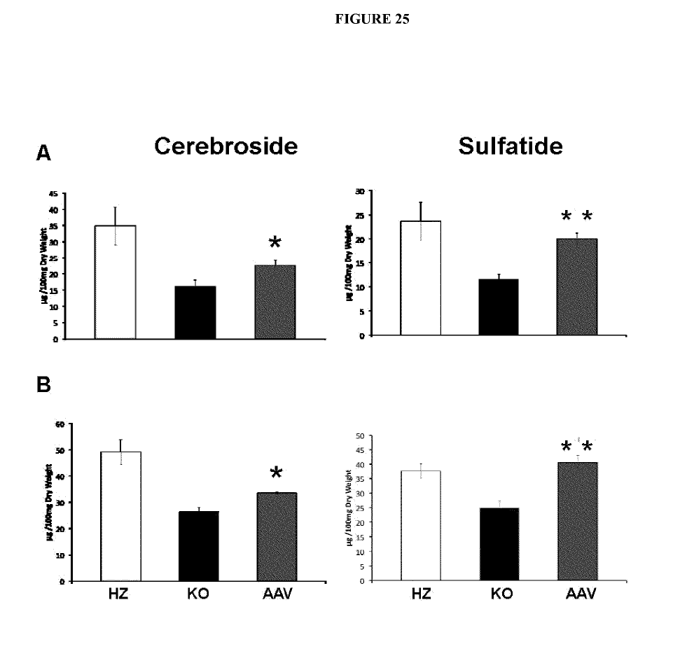

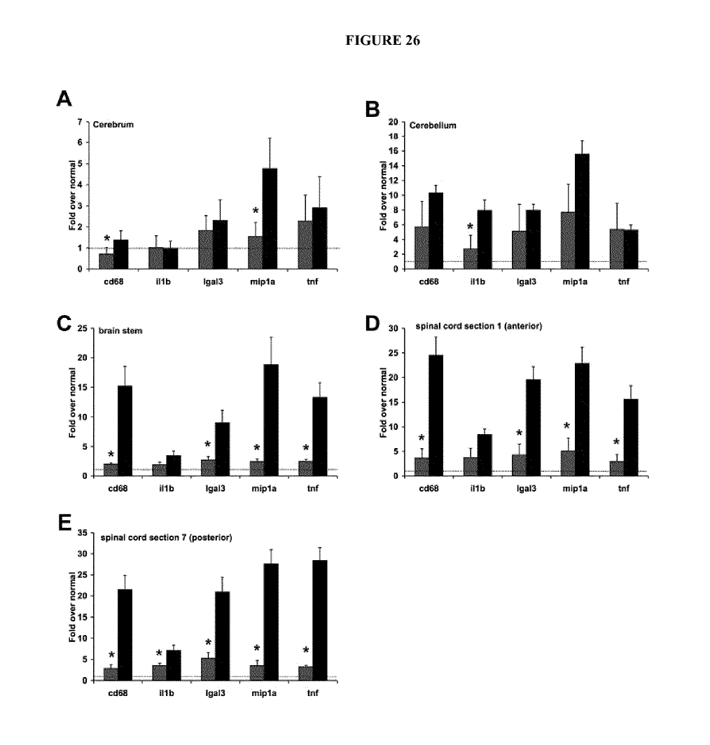

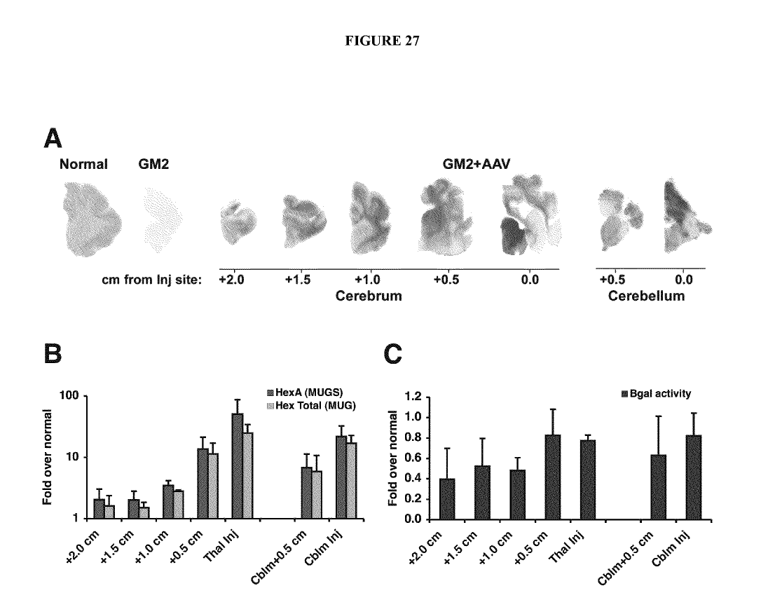

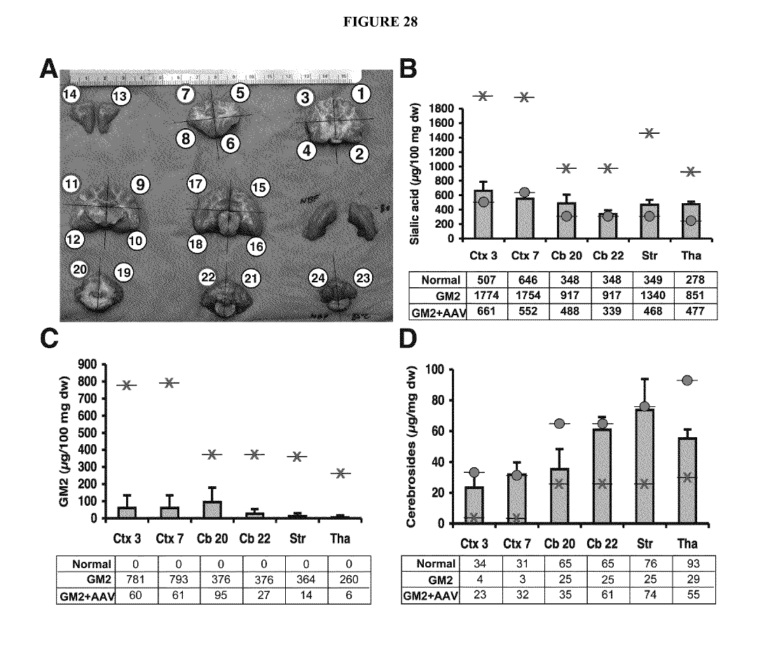

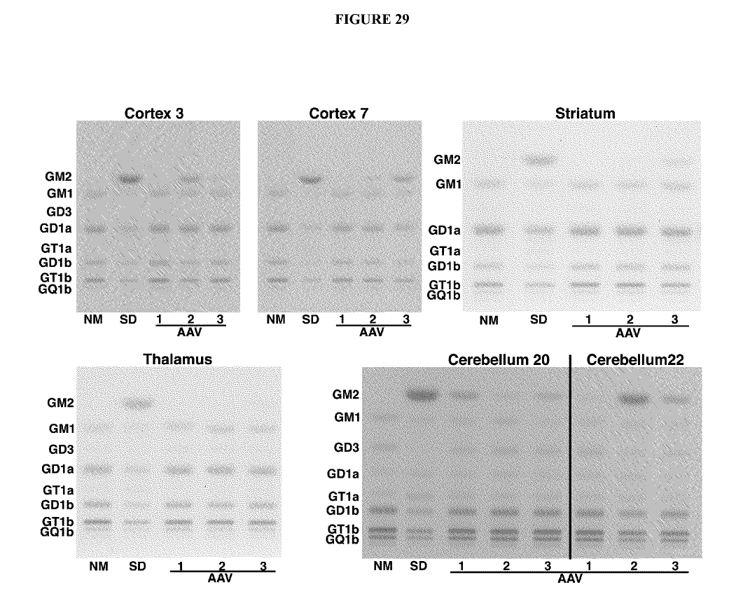

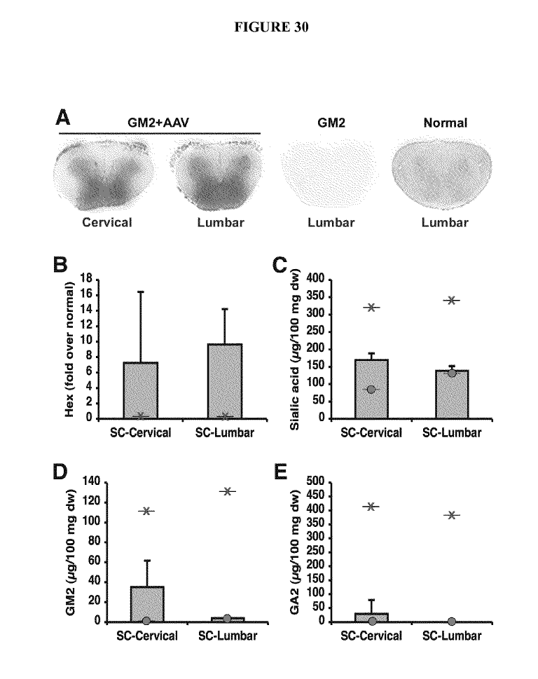

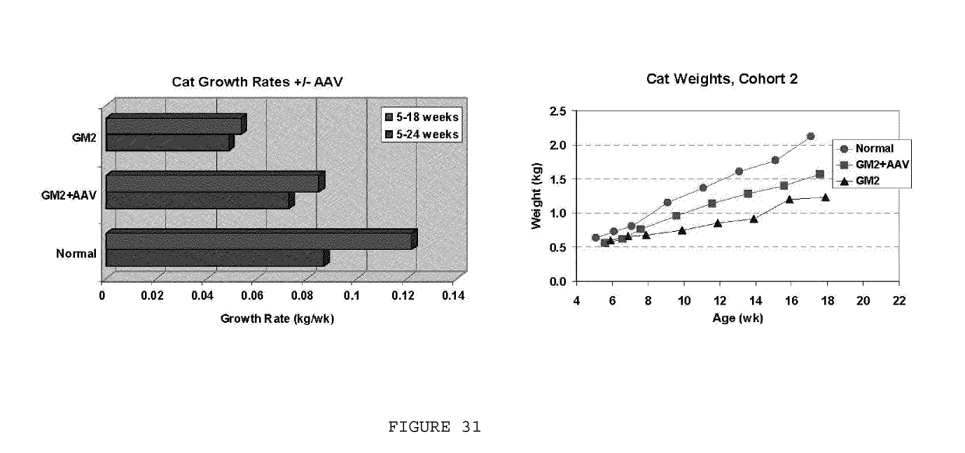

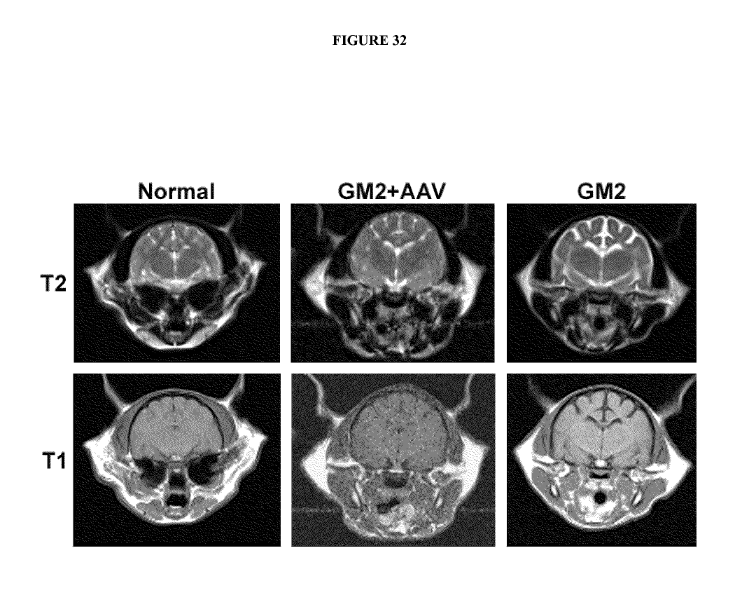

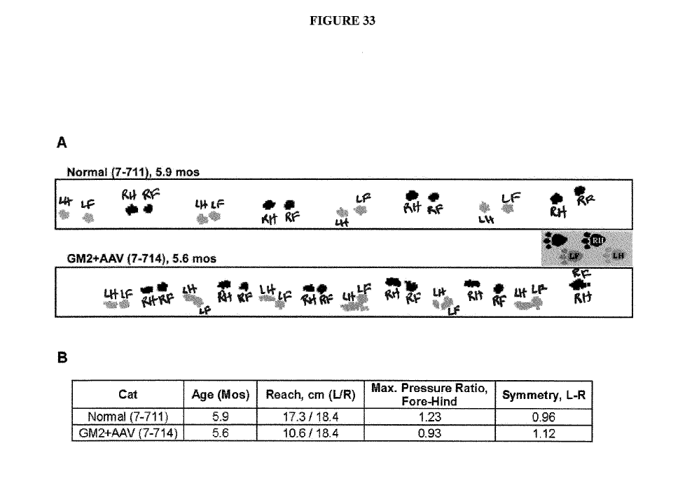

GM1-gangliosidosis is a neurodegenerative lysosomal storage disease caused by deficiency of acid β-galactosidase (β-gal) leading to progressive accumulation of GM1-ganglioside in the CNS (FIG. 1). Age of onset of the symptoms ranges from infancy to adulthood and the severity of the clinical manifestations mostly correlates with the levels of residual enzyme activity. In the most severe form of this disease (Infantile or Type I) biochemical and neuropathological alterations have been documented in utero. Progressive neurologic deterioration, macular cherry red spot, facial dysmorphism, hepatosplenomegaly, generalized skeletal dysplasia and early death are common features of the disease.

The available knockout mouse models replicate several clinical and biochemical features of infantile GM1-gangliosidosis with low levels of βgal activity (<4% of normal) and massive accumulation of GM1-ganglioside and GA1 glycosphingolipid throughout the CNS. The βgal^{-/-} (GM1) mice accumulate abnormal levels of GM1-ganglioside as early as post-natal day 5, and reach several fold above normal by 3 months of age. This feature is associated with a progressively severe CNS condition characterized by tremor, ataxia, abnormal gait and ultimately paralysis of the hind limbs. Studies on this mouse model identified previously unknown molecular pathways that are induced by GM1 accumulation and result in neuronal apoptosis and neurodegeneration. Defective lysosomal degradation of GM1 was found to provoke the redistribution of this ganglioside at the ER membranes, where it induces depletion of ER Ca²⁺ stores, and in turn activation of the unfolded protein response (UPR) and UPR-mediated apoptosis. More recently it was shown that GM1 accumulates specifically in glycosphingolipid-enriched fractions (GEMs) of the mitochondria-associated ER membranes, the sites of apposition between ER and mitochondria GM1 at the GEMs favors Ca²⁺ flux between these organelles, which results in mitochondrial Ca²⁺ overload and activation of the mitochondrial leg of apoptosis. Neuronal apoptosis is accompanied by neuroinflammation with increased microglial activation, production of inflammatory cytokines, chemokines, and inflammatory cell infiltration.

Currently there is no effective treatment for GM1-gangliosidosis in children, although numerous therapeutic modalities have been implemented in GM1 mice with somewhat encouraging results.

### GM2 Gangliosidosis

Tay-Sachs and Sandhoff Diseases comprise a subset of neuronopathic LSDs characterized by storage of GM2 ganglioside in the CNS. These ‘GM2 gangliosidoses’ result from inherited defects in the lysosomal glycohydrolase, β-N-acetylhexosaminidase (Hex). The prevalence of Tay-Sachs disease (TSD) and Sandhoff disease (SD) in the general population is ˜1 in 100,000 live births for each disease, while carrier frequency in the general population is 1 in 167 for TSD and 1 in 278 for SD. However, carrier frequencies may be 100-fold higher in certain ethnic groups, such as Ashkenazi Jews, Cajuns in southern Louisiana, French Canadians in eastern Quebec, or the Pennsylvania Dutch. Having very similar clinical phenotypes in humans, TSD and SD are characterized by relentlessly progressive nervous system dysfunction and are classified according to disease severity as (1) infantile, (2) juvenile or (3) adult-onset forms. Babies with the infantile (most severe) form develop normally for the first 3-6 months of life, after which development slows and then begins to regress. A stereotypical “cherry red spot” is evident on the fundus of the retina, created by retinal neurons grossly swollen with ganglioside storage material. By age two, affected children suffer from frequent seizures, swallowing difficulties, respiratory infections, and loss of motor control. Death typically occurs before the fifth birthday. Late-onset GM2 gangliosidosis is the second most common form, but because early symptoms are common to other diseases, affected adults may be misdiagnosed for >10 years. Symptoms typically include speech difficulties, muscle weakness, tremor and ataxia. Manic-depressive or psychotic episodes are present in about 30% of affected persons. The majority of late-onset patients are wheelchair-bound by age 30-40. The juvenile forms vary greatly in severity from case to case, but all juvenile forms of GM2 gangliosidosis are fatal. Although GM2 gangliosidosis was first described more than a century ago, it remains largely untreatable.

β-Hexosaminidase is composed of 2 subunits, α and β, encoded respectively by the HEXA and HEXB genes. HEXA mutations cause TSD, while defects in HEXB produce SD, both resulting in abnormal storage of GM2 ganglioside in the CNS. GM2 ganglioside is degraded by the coordinated action of 3 gene products: the α and β subunits of hexosaminidase and the GM2 activator protein, a non-degradative accessory protein necessary for ganglioside presentation to the Hex enzyme. Hex subunits dimerize to form separate isozymes with different substrate specificities: HexA (αβ), HexB (ββ) and HexS (αα) (an unstable isoform present at very low levels). Only those isozymes containing the α-subunit are capable of appreciable GM2-ganglioside degradation. Therefore, HexA is the predominant isozyme responsible for clearance of GM2 ganglioside, and its function may be eliminated by defects in either the α- or β-subunit. The α- and β-subunit precursor proteins are translated and translocated into the lumen of the endoplasmic reticulum (ER), where they dimerize to form immature HexA and HexB molecules. A pool of excess α-subunit monomer is maintained for at least 5 hours in the ER and thought to force formation of the less stable HexA (αβ) isozyme through mass action, since the ββ homodimer (HexB) is more stable and more readily formed. If subunit dimerization does not occur, monomers appear to be retained in the ER and degraded. Final acquisition of the mannose-6-phosphate signal for lysosomal targeting occurs in the Golgi, and proteolytic processing to the mature isozymes occurs in the lysosome. Therefore, subunit dimerization in the ER is a first step toward ultimate isozyme maturation in the lysosome, for enzymatic activity toward GM2 ganglioside.

Gene therapy approaches for GM2-gangliosidoses take into account that simple overexpression of α- or β-subunits individually will create an imbalance in the intracellular stoichiometry of α- and β-subunits. Gene transfer experiments in cell culture have shown that overexpression of human α-subunit in human or mouse Tay-Sachs fibroblasts produces a significant reduction in HexB activity, presumably by depletion of the endogenous β-subunit pool. Also gene transfer experiments in Tay-Sachs mice have shown that co-transduction with two viral vectors encoding human α- and β-subunit separately to achieve high-level HexA synthesis and secretion. Therefore effective gene therapy strategies for GM2-gangliosidoses should utilize gene delivery vehicles encoding both the α- and β-subunits.

Knockout mouse models of TSD were reported by separate laboratories in 1995 and 1996. By disruption of HEXA and HEXB genes in embryonic stem cells, the authors recapitulated the biochemical defects of TSD and SD, respectively. Although human clinical phenotypes are very severe and almost identical for the two disorders, HEXA-knockout mice display very mild neurological abnormalities appearing at >1 year of age while HEXB-knockout mice are severely affected, with disease onset at≈3 months. Survival times are≈2 years of age (a normal lifespan) for HEXA-knockout mice and≈4 months for HEXB-knockout mice. By studying these phenotypically dissimilar knockout mice, an alternative catabolic pathway for gangliosides was discovered that is not physiologically relevant in humans.

Human ganglioside catabolism (designated the “classic” pathway) is dependent upon HexA cleavage of GM2 ganglioside (FIG. 1). Although mice also utilize the classic pathway, an alternative pathway exists in which HexB plays a minor role in ganglioside degradation. Therefore, in the absence of α-subunit and the HexA isozyme, the classic pathway for GM2 catabolism is abolished while the alternative pathway remains functional, albeit at reduced efficiency. Severe clinical disease results from α- or β-subunit deficiency in humans since the classic pathway is abolished in either case, but only β-subunit deficiency produces severe disease in mice because both pathways are dysfunctional. Therefore, therapeutic experiments designed to evaluate clinical benefit through phenotypic improvement are typically performed in HEXB-knockout mice.

The feline GM2 gangliosidosis model has been maintained since 1991 at the Scott-Ritchey Research Center, College of Veterinary Medicine, Auburn University (Alabama). Cats with GM2 gangliosidosis variant 0 (Sandhoff Disease) display clinical and histopathological features typical of the human disease, with a slight head and body tremor beginning at 8±1 weeks that progresses to inability to ambulate, use the litter box or eat without assistance from care givers by 17±2 weeks of age. Weight loss of 20% maximal body weight defines the humane endpoint, reached at 19.3±2.0 weeks. Stereotypical disease progression provides an excellent opportunity to evaluate therapeutic benefit in treated animals. Membranous cytoplasmic bodies, meganeurites and ectopic neurites are histopathological features common to both human and feline gangliosidoses, with storage identified clearly by thin layer chromatography or special stains such as periodic acid-Schiff (PAS). The cat brain, which is 75 times larger than the mouse brain and more similar in organization and complexity to the human brain, provides a good approximation of the vector/enzyme distribution challenges to be overcome for human CNS gene therapy. Additionally, the cat model replicates the human condition, with behavioral abnormalities and disease progression that can be evaluated clinically as well as biochemically after treatment.

Recent studies have determined that, just as cat brain size and complexity are intermediate between mice and humans, cats provide an intermediate model of ganglioside catabolism as well. For example, while GM2 comprises only 38% of cerebral cortical gangliosides in HEXB-knockout mice, it constitutes 67% and 85% of cortical gangliosides in affected cats and humans, respectively. Therefore, therapeutic experiments in affected cats are expected to be a critical step in translation of AAV vector-mediated therapy to human clinical trials.

### Lysosomal Storage Disorder Therapies

Although TSD was first described in 1881 and the precise enzymatic deficiency was identified in the late 1960's, it remains untreatable today. However, a number of observations have been made which encourage continuing efforts to develop effective therapy for lysosomal diseases such as TSD and SD. For example, tissues of individuals heterozygous for the gangliosidoses, who have no clinical signs of disease, can have as little as 15-20% of normal tissue enzyme activity, and patients with late onset disease and reduced severity of clinical signs have only 1-5% of normal enzyme activity, indicating that restoration of minimal functional enzyme activity may be adequate to prevent or reduce disease severity. Neufeld and coworkers first demonstrated that lysosomal enzymes secreted from normal cells are endocytosed by mutant cells with correction of the metabolic defect, suggesting that enzyme donor cells which constitute only a portion of an organ might effect restoration of lysosomal function in a larger population of cells. Discovery of this “cross-correction” mechanism in the mid 1970s stimulated the development of methods to replace missing enzymes in the various LSDs. This mechanism is the basis for all existing therapies for LSDs, including enzyme replacement therapy (ERT), which has been approved for treating a number of LSDs.

In humans, ERT has proven ineffective to treat the brain in LSDs with neurological features because the blood-brain barrier (BBB) restricts entry of peripherally infused enzymes into the brain. Experimental approaches to treat neuronopathic LSDs center on 4 main strategies: 1) Gene Therapy; 2) Substrate reduction therapy; 3) Enzyme enhancement (or Chaperone) therapy; 4) Stem cell therapy.

Adeno-associated virus (AAV) vectors have become the vectors of choice for gene delivery to the brain because of their exceptional efficiency in transducing neurons where they promote long-term expression of therapeutic genes with no apparent toxicity, and limited inflammation at the site of injection. Direct infusion of AAV vectors into the brain parenchyma has shown remarkable therapeutic efficacy in a large number of mouse models of LSDs with neurological features, including GM2-gangliosidoses. Also this approach has been tested in α-mannosidosis cats and mucopolysaccharidosis type I (MPS I) dogs with very promising results. Two AAV2/1-treated α-mannosidosis cats showed clear improvement, with one cat surviving past 1 year of age with relatively minor symptoms. In AAV5-treated MSP I dogs there was enzymatic activity in many regions of the brain (>90% in two dogs), and a marked reduction in lysosomal storage metabolites. Exemplary disorders for which AAV-mediated gene therapy as been demonstrated to be effective is shown in Table 1.

TABLE 1
List of diseases where AAV-mediated gene delivery to
the brain of an animal has demonstrated efficacy.

| Disease | References |
|---|---|
| Mucopolysaccharidosis type VII | 1-9 |
| Mucopolysaccharidosis type I | 10, 11 |
| Mucopolysaccharidosis type IIIB | 12, 13 |
| Niemann-Pick A | 14, 15 |
| Krabbe Disease | 16, 17 |
| Infantile neuronal ceroid lipofuscinosis | 18, 23 |
| Metachromatic leukodystrophy | 24, 25 |
| GM1-gangliosidosis | 29 |
| GM2-gangliosidosis (Tay-Sachs and Sandhoff Diseases) | 27 |
| α-mannosidosis | 30 |

AAV2 vectors have been injected into the human brain in clinical trials for Parkinson's disease, and children with Canavan disease or late infantile neuronal ceroid lipofuscinosis. Data from these clinical trials suggest that AAV2 vectors are safe for gene delivery to the human brain. AAV-treated Parkinson's patients showed sustained improvements in motor scores out to 1 year post-infusion, and positron emission tomography findings showed a consistent decrease in activity of the motor-control network in the brain. These early findings from clinical trials suggest that AAV-mediated gene expression in the human brain is safe and likely to be as long lasting and stable as in the brains of other species.

AAV vector technology has evolved sufficiently to achieve efficient genetic modification of the thalamus in humans to create an enzyme producing ‘central node’ capable of distributing functional enzyme throughout the entire brain, and thus alter the course of disease progression in Tay-Sachs and Sandhoff diseases. This strategy should be applicable to many, if not all other LSDs with neurological involvement where the therapeutic protein can be secreted from genetically modified cells and taken up by diseased cells.

Direct infusion of adeno-associated virus (AAV) vectors encoding lysosomal enzymes into the brain parenchyma has emerged as a viable strategy to create an in situ source of normal enzyme in the brain. An obstacle to translation of the promising results obtained in animal models is the number of injections that may be needed to achieve global distribution of enzyme throughout the human brain. Based on studies in α-mannosidosis cats, it has been estimated that 40-60 injections of AAV vector may be necessary to obtain global distribution of lysosomal enzymes in the infant brain. This large number of injections makes the treatment extremely invasive with obvious risks. Therefore, alternative strategies are needed.

The present disclosure relates generally to methods for the treatment of lysosomal storage disorders with AAV-mediated gene therapy. In particular, the present disclosure provides methods for treating, reducing the severity of, or delaying the onset of Tay-Sachs Disease and Sandhoff Disease by providing AAV-mediated HexA expression in the brain of a subject in need thereof. AAV-mediated HexA expression is achieved by administering pharmaceutical compositions comprising AAV-HexA expression constructs to the brain of the subject. Typically, the methods result in widespread distribution of a therapeutic enzyme in the brain of the subject.

### Preparation of AAV Expression Constructs

AAV expression constructs disclosed herein may be constructed using methodologies known in the art of molecular biology. The descriptions herein are to be construed as exemplary and not limiting. Typically, AAV vectors carrying enzyme coding sequences are assembled from polynucleotides comprising constituent parts of the AAV expression construct.

One method of obtaining constituent parts of an AAV expression construct is polymerase chain reaction (PCR) amplification of nucleic acids. Methods for PCR are taught in MacPherson, et al. “PCR: A Practical Approach,” IRL Press as Oxford University Press, (1991). Specific reaction conditions for amplification of desired sequences may be empirically determined. A number of parameters effect the efficiency of amplification, including annealing temperature, annealing time, extension time, Mg²⁺ and ATP concentrations, pH, and the relative concentrations of templates, primers, polymerase, and deoxyribonucleotides. Reaction products can be evaluated by agarose gel electrophoresis and ethidium bromide staining.

Another method for constructing AAV expression vectors is enzymatic digestion. Nucleotides sequences can be generated by digestion of appropriate vectors or PCR products with restriction enzymes. Digested fragments may be ligated together as appropriate.

Polynucleotides are inserted into AAV genomes using methods known in the art. For example, DNA may be contacted with suitable restriction enzymes to generate fragments with ends that are complementary and compatible for joining. Additionally or alternatively, synthetic linkers may be ligated to the end(s) of an existing fragment to render it compatible for ligation with another fragment and/or a vector.

AAV expression constructs may be amplified by transfection of an appropriate host cell, such as HEK 293 cells. The amplified construct may be isolated from host cells and purified for use in the methods disclosed herein using methods known in the art.

In the disclosed methods, the viral vector used to distribute replacement enzymes in the brain of subjects is an AAVrh.8 vector. By “AAVrh.8” is meant a vector derived from adeno-associated virus serotype AAVrh.8, which is described in Maguire, et al., 16 Mol. Ther. 1695-1702 (2008) and Gao, et al., 78(12) J. Virol. 6381-6388 (2004). The term encompasses natural or engineered derivatives of AAVrh.8 with substantial identity to SEQ ID NO:1.

| | |
|---|---|
| 1 | atggctgccg atggttatct tccagattgg ctcgaggaca acctctctga gggcattcgc |
| 61 | gagtggtggg acttgaaacc tggagccccg aaacccaaag ccaaccagca aaagcaggac |
| 121 | gacggccggg gtctggtgct tcctggctac aagtacctcg gacccttcaa cggactcgac |
| 181 | aagggggagc ccgtcaacgc ggcggacgca gcggccctcg agcacgacaa agcctacgac |
| 241 | cagcagctca aagcgggtga caatccgtac ctgcggtata atcacgccga cgccgagttt |
| 301 | caggagcgtc tgcaagaaga tacgtctttt gggggcaacc tcgggcgagc agtcttccag |
| 361 | gccaagaagc gggttctcga acctctcggt ctggttgagg aaggcgctaa gacggctcct |
| 421 | ggaaagaaga gaccggtaga gcagtcgcca caagagccag actcctcctc gggcatcggc |
| 481 | aagacaggcc agcagcccgc taaaaagaga ctcaattttg gtcagactgg cgactcagag |
| 541 | tcagtccccg acccacaacc tctcggagaa cctccagcag ccccctcagg tctgggacct |
| 601 | aatacaatgg cttcaggcgg tggcgctcca atggcagaca ataacgaagg cgccgacgga |
| 661 | gtgggtaatt cctcgggaaa ttggcattgc gattccacat ggctggggga cagagtcatc |
| 721 | accaccagca cccgaacctg ggccctgccc acctacaaca accacctcta caagcaaatc |
| 781 | tccaacggca cctcgggagg aagcaccaac gacaacacct attttggcta cagcaccccc |
| 841 | tgggggtatt ttgacttcaa cagattccac tgtcactttt caccacgtga ctggcaacga |
| 901 | ctcatcaaca acaattgggg attccggccc aaaagactca acttcaagct gttcaacatc |
| 961 | caggtcaagg aagtcacgac gaacgaaggc accaagacca tcgccaataa tctcaccagc |
| 1021 | accgtgcagg tctttacgga ctcggagtac cagttaccgt acgtgctagg atccgctcac |
| 1081 | cagggatgtc tgcctccgtt cccggcggac gtcttcatgg ttcctcagta cggctattta |
| 1141 | actttaaaca atggaagcca agccctggga cgttcctcct tctactgtct ggagtatttc |
| 1201 | ccatcgcaga tgctgagaac cggcaacaac tttcagttca gctacacctt cgaggacgtg |
| 1261 | cctttccaca gcagctacgc gcacagccag agcctggaca ggctgatgaa tcccctcatc |
| 1321 | gaccagtacc tgtactacct ggtcagaacg caaacgactg gaactggagg gacgcagact |
| 1381 | ctggcattca gccaagcggg tcctagctca atggccaacc aggctagaaa ttgggtgccc |
| 1441 | ggaccttgct accggcagca gcgcgtctcc acgacaacca accagaacaa caacagcaac |
| 1501 | tttgcctgga cgggagctgc caagtttaag ctgaacggcc gagactctct aatgaatccg |
| 1561 | ggcgtggcaa tggcttccca caaggatgac gacgaccgct tcttcccttc gagcggggtc |
| 1621 | ctgatttttg gcaagcaagg agccgggaac gatggagtgg attacagcca agtgctgatt |
| 1681 | acagatgagg aagaaatcaa ggctaccaac cccgtggcca cagaagaata tggagcagtg |
| 1741 | gccatcaaca accaggccgc caatacgcag gcgcagaccg gactcgtgca caaccagggg |
| 1801 | gtgattcccg gcatggtgtg gcagaataga gacgtgtacc tgcagggtcc catctgggcc |
| 1861 | aaaattcctc acacggacgg caactttcac ccgtctcccc tgatgggcgg ctttggactg |
| 1921 | aagcacccgc ctcctcaaat tctcatcaag aacacaccgg ttccagcgga cccgccgctt |
| 1981 | accttcaacc aggccaagct gaactctttc atcacgcagt acagcaccgg acaggtcagc |
| 2041 | gtggaaatcg agtgggagct gcagaaagaa aacagcaaac gctggaatcc agagattcaa |
| 2101 | tacacttcca actactacaa atctacaaat gtggactttg ctgtcaacac ggagggggtt |
| 2161 | tatagcgagc ctcgccccat tggcacccgt tacctcaccc gcaacctgta a |

In some embodiments, the AAV expression vector comprises the coding sequence of acid β-galactosidase. In some embodiments, the AAV expression vector comprises independent constructs encoding the β-hexosaminidase α and β subunits. Constructs encoding a particular enzymes may be constructed by cloning enzyme coding sequences into an AAVrh.8 vector using molecular biology methods known in the art. Cloning methods may be adapted accordingly based on the specific nucleotide sequences being manipulated.

An alternate viral vector encompasses natural or engineered derivatives of AAVrh.8 with substantial identity to SEQ ID NO:2.

| | |
|---|---|
| 1 | atgccggggt tttacgagat tgtgattaag gtccccagcg accttgacgg gcatctgccc |
| 61 | ggcatttctg acagctttgt gaactgggtg gccgagaagg aatgggagtt gccgccagat |
| 121 | tctgacatgg atctgaatct gattgagcag gcacccctga ccgtggccga gaagctgcag |
| 181 | cgcgactttc tgacggaatg gcgccgtgtg agtaaggccc cggaggccct tttctttgtg |
| 241 | caatttgaga agggagagag ctacttccac atgcacgtgc tcgtggaaac caccggggtg |
| 301 | aaatccatgg ttttgggacg tttcctgagt cagattcgcg aaaaactgat tcagagaatt |
| 361 | taccgcggga tcgagccgac tttgccaaac tggttcgcgg tcacaaagac cagaaatggc |
| 421 | gccggaggcg ggaacaaggt ggtggatgag tgctacatcc ccaattactt gctccccaaa |
| 481 | acccagcctg agctccagtg ggcgtggact aatatggaac agtatttaag cgcctgtttg |
| 541 | aatctcacgg agcgtaaacg gttggtggcg cagcatctga cgcacgtgtc gcagacgcag |
| 601 | gagcagaaca aagagaatca gaatcccaat tctgatgcgc cggtgatcag atcaaaaact |
| 661 | tcagccaggt acatggagct ggtcgggtgg ctcgtggaca aggggattac ctcggagaag |
| 721 | cagtggatcc aggaggacca ggcctcatac atctccttca atgcggcctc caactcgcgg |
| 781 | tcccaaatca aggctgcctt ggacaatgcg ggaaagatta tgagcctgac taaaaccgcc |
| 841 | cccgactacc tggtgggcca gcagcccgtg gaggacattt ccagcaatcg gatttataaa |
| 901 | attttggaac taaacgggta cgatccccaa tatgcggctt ccgtctttct gggatgggcc |
| 961 | acgaaaaagt tcggcaagag gaacaccatc tggctgtttg ggcctgcaac taccgggaag |
| 1021 | accaacatcg cggaggccat agcccacact gtgcccttct acgggtgcgt aaactggacc |
| 1081 | aatgagaact ttcccttcaa cgactgtgtc gacaagatgg tgatctggtg ggaggagggg |
| 1141 | aagatgaccg ccaaggtcgt ggagtcggcc aaagccattc tcggaggaag caaggtgcgc |
| 1201 | gtggaccaga aatgcaagtc ctcggcccag atagacccga ctcccgtgat cgtcacctcc |
| 1261 | aacaccaaca tgtgcgccgt gattgacggg aactcaacga ccttcgaaca ccagcagccg |
| 1321 | ttgcaagacc ggatgttcaa atttgaactc acccgccgtc tggatcatga ctttgggaag |
| 1381 | gtcaccaagc aggaagtcaa agactttttc cggtgggcaa aggatcacgt ggttgaggtg |
| 1441 | gagcatgaat tctacgtcaa aaagggtgga gccaagaaaa gacccgcccc cagtgacgca |
| 1501 | gatataagtg agcccaaacg ggtgcgcgag tcagttgcgc agccatcgac gtcagacgcg |
| 1561 | gaagcttcga tcaactacgc agacaggtac caaaacaaat gttctcgtca cgtgggcatg |
| 1621 | aatctgatgc tgtttccctg cagacaatgc gagagaatga atcagaattc aaatatctgc |
| 1681 | ttcactcacg gacagaaaga ctgtttagag tgctttcccg tgtcagaatc tcaacccgtt |
| 1741 | tctgtcgtca aaaaggcgta tcagaaactg tgctacattc atcatatcat gggaaaggtg |
| 1801 | ccagacgctt gcactgcctg cgatctggtc aatgtggatt tggatgactg catctttgaa |
| 1861 | caataaatga tttaaatcag gtatggctgc cgatggttat cttccagatt ggctcgagga |
| 1921 | caacctctct gagggcattc gcgagtggtg ggacttgaaa cctggagccc cgaagcccaa |
| 1981 | agccaaccag caaaagcagg acgacggccg gggtctggtg cttcctggct acaagtacct |
| 2041 | cggacccttc aacggactcg acaaggggga gcccgtcaac gcggcggacg cagcggccct |
| 2101 | cgagcacgac aaggcctacg accagcagct caaagcgggt gacaatccgt acctgcggta |
| 2161 | taaccacgcc gacgccgagt ttcaggagcg tctgcaagaa gatacgtctt ttgggggcaa |
| 2221 | cctcgggcga gcagtcttcc aggccaagaa gcgggttctc gaacctctcg gtctggttga |
| 2281 | ggaaggcgct aagacggctc ctggaaagaa acgtccggta gagcagtcgc cacaagagcc |
| 2341 | agactcctcc tcgggcatcg gcaagacagg ccagcagccc gctaaaaaga gactcaattt |
| 2401 | tggtcagact ggcgactcag agtcagtccc cgatccacaa cctctcggag aacctccagc |
| 2461 | agccccctca ggtctgggac ctaatacaat ggcttcaggc ggtggcgctc caatggcaga |
| 2521 | caataacgaa ggcgccgacg gagtgggtaa ttcctcggga aattggcatt gcgattccac |
| 2581 | atggctgggg gacagagtca tcaccaccag cacccgaacc tgggccctgc ccacctacaa |
| 2641 | caaccacctc tacaagcaaa tctccaacgg cacctcggga ggaagcacca acgacaacac |
| 2701 | ctattttggc tacagcaccc cctgggggta ttttgacttc aacagattcc actgtcactt |
| 2761 | ttcaccacgt gactggcaac gactcatcaa caacaattgg ggattccggc ccaaaagact |
| 2821 | caacttcaag ctgttcaaca tccaggtcaa ggaagtcacg acgaacgaag gcaccaagac |
| 2881 | catcgccaat aatctcacca gcaccgtgca ggtctttacg gactcggagt accagttacc |
| 2941 | gtacgtgcta ggatccgctc accagggatg tctgcctccg ttcccggcgg acgtcttcat |
| 3001 | ggttcctcag tacggctatt taactttaaa caatggaagc caagccctgg gacgttcctc |
| 3061 | cttctactgt ctggagtatt tcccatcgca gatgctgaga accggcaaca actttcagtt |
| 3121 | cagctacacc ttcgaggacg tgcctttcca cagcagctac gcgcacagcc agagcctgga |
| 3181 | caggctgatg aatcccctca tcgaccagta cctgtactac ctggtcagaa cgcaaacgac |
| 3241 | tggaactgga gggacgcaga ctctggcatt cagccaagcg ggtcctagct caatggccaa |
| 3301 | ccaggctaga aattgggtgc ccggaccttg ctaccggcag cagcgcgtct ccacgacaac |
| 3361 | caaccagaac aacaacagca actttgcctg gacgggagct gccaagttta agctgaacgg |
| 3421 | ccgagactct ctaatgaatc cgggcgtggc aatggcttcc cacaaggatg acgacgaccg |
| 3481 | cttcttccct tcgagcgggg tcctgatttt tggcaagcaa ggagccggga acgatggagt |
| 3541 | ggattacagc caagtgctga ttacagatga ggaagaaatc aaggctacca accccgtggc |
| 3601 | cacagaagaa tatggagcag tggccatcaa caaccaggcc gccaatacgc aggcgcagac |
| 3661 | cggactcgtg cacaaccagg gggtgattcc cggcatggtg tggcagaata gagacgtgta |
| 3721 | cctgcagggt cccatctggg ccaaaattcc tcacacggac ggcaactttc acccgtctcc |
| 3781 | cctgatgggc ggctttggac tgaagcaccc gcctcctcaa attctcatca agaacacacc |
| 3841 | ggttccagcg gacccgccgc ttaccttcaa ccaggccaag ctgaactctt tcatcacgca |
| 3901 | gtacagcacc ggacaggtca gcgtggaaat cgagtgggag ctgcagaaag aaaacagcaa |
| 3961 | acgctggaat ccagagattc aatacacttc caactactac aaatctacaa atgtggactt |
| 4021 | tgctgtcaac acggaggggg tttatagcga gcctcgcccc attggcaccc gttacctcac |
| 4081 | ccgcaacctg taattacgtg ttaatcaata aaccggttga ttcgtttcag ttgaactttg |
| 4141 | gtgtcgcggc cgctcgataa gcttttgttc cctttagtga gggttaattc cgagcttggc |
| 4201 | gtaatcatgg tcatagctgt ttcctgtgtg aaattgttat ccgctcacaa ttccacacaa |
| 4261 | catacgagcc ggaagcataa agtgtaaagc ctggggtgcc taatgagtga gctaactcac |
| 4321 | attaattgcg ttgcgctcac tgcccgcttt ccagtcggga aacctgtcgt gccagctgca |
| 4381 | ttaatgaatc ggccaacgcg cggggagagg cggtttgcgt attgggcgct cttccgcttc |
| 4441 | ctcgctcact gactcgctgc gctcggtcgt tcggctgcgg cgagcggtat cagctcactc |
| 4501 | aaaggcggta atacggttat ccacagaatc aggggataac gcaggaaaga acatgtgagc |
| 4561 | aaaaggccag caaaaggcca ggaaccgtaa aaaggccgcg ttgctggcgt ttttccatag |
| 4621 | gctccgcccc cctgacgagc atcacaaaaa tcgacgctca agtcagaggt ggcgaaaccc |
| 4681 | gacaggacta taaagatacc aggcgtttcc ccctggaagc tccctcgtgc gctctcctgt |
| 4741 | tccgaccctg ccgcttaccg gatacctgtc cgcctttctc ccttcgggaa gcgtggcgct |
| 4801 | ttctcatagc tcacgctgta ggtatctcag ttcggtgtag gtcgttcgct ccaagctggg |
| 4861 | ctgtgtgcac gaaccccccg ttcagcccga ccgctgcgcc ttatccggta actatcgtct |
| 4921 | tgagtccaac ccggtaagac acgacttatc gccactggca gcagccactg gtaacaggat |
| 4981 | tagcagagcg aggtatgtag gcggtgctac agagttcttg aagtggtggc ctaactacgg |
| 5041 | ctacactaga aggacagtat ttggtatctg cgctctgctg aagccagtta ccttcggaaa |
| 5101 | aagagttggt agctcttgat ccggcaaaca aaccaccgct ggtagcggtg gtttttttgt |
| 5161 | ttgcaagcag cagattacgc gcagaaaaaa aggatctcaa gaagatcctt tgatcttttc |
| 5221 | tacggggtct gacgctcagt ggaacgaaaa ctcacgttaa gggattttgg tcatgagatt |
| 5281 | atcaaaaagg atcttcacct agatcctttt aaattaaaaa tgaagtttta aatcaatcta |
| 5341 | aagtatatat gagtaaactt ggtctgacag ttaccaatgc ttaatcagtg aggcacctat |
| 5401 | ctcagcgatc tgtctatttc gttcatccat agttgcctga ctccccgtcg tgtagataac |
| 5461 | tacgatacgg gagggcttac catctggccc cagtgctgca atgataccgc gagacccacg |
| 5521 | ctcaccggct ccagatttat cagcaataaa ccagccagcc ggaagggccg agcgcagaag |
| 5581 | tggtcctgca actttatccg cctccatcca gtctattaat tgttgccggg aagctagagt |
| 5641 | aagtagttcg ccagttaata gtttgcgcaa cgttgttgcc attgctacag gcatcgtggt |
| 5701 | gtcacgctcg tcgtttggta tggcttcatt cagctccggt tcccaacgat caaggcgagt |
| 5761 | tacatgatcc cccatgttgt gcaaaaaagc ggttagctcc ttcggtcctc cgatcgttgt |
| 5821 | cagaagtaag ttggccgcag tgttatcact catggttatg gcagcactgc ataattctct |
| 5881 | tactgtcatg ccatccgtaa gatgcttttc tgtgactggt gagtactcaa ccaagtcatt |
| 5941 | ctgagaatag tgtatgcggc gaccgagttg ctcttgcccg gcgtcaatac gggataatac |
| 6001 | cgcgccacat agcagaactt taaaagtgct catcattgga aaacgttctt cggggcgaaa |
| 6061 | actctcaagg atcttaccgc tgttgagatc cagttcgatg taacccactc gtgcacccaa |
| 6121 | ctgatcttca gcatctttta ctttcaccag cgtttctggg tgagcaaaaa caggaaggca |
| 6181 | aaatgccgca aaaaagggaa taagggcgac acggaaatgt tgaatactca tactcttcct |
| 6241 | ttttcaatat tattgaagca tttatcaggg ttattgtctc atgagcggat acatatttga |
| 6301 | atgtatttag aaaaataaac aaataggggt tccgcgcaca tttccccgaa aagtgccacc |
| 6361 | tgacgtctaa gaaaccatta ttatcatgac attaacctat aaaaataggc gtatcacgag |
| 6421 | gccctttcgt ctcgcgcgtt tcggtgatga cggtgaaaac ctctgacaca tgcagctccc |
| 6481 | ggagacggtc acagcttgtc tgtaagcgga tgccgggagc agacaagccc gtcagggcgc |
| 6541 | gtcagcgggt gttggcgggt gtcggggctg gcttaactat gcggcatcag agcagattgt |
| 6601 | actgagagtg caccatatgc ggtgtgaaat accgcacaga tgcgtaagga gaaaataccg |
| 6661 | catcaggaaa ttgtaaacgt taatattttg ttaaaattcg cgttaaattt ttgttaaatc |
| 6721 | agctcatttt ttaaccaata ggccgaaatc ggcaaaatcc cttataaatc aaaagaatag |
| 6781 | accgagatag ggttgagtgt tgttccagtt tggaacaaga gtccactatt aaagaacgtg |
| 6841 | gactccaacg tcaaagggcg aaaaaccgtc tatcagggcg atggcccact acgtgaacca |
| 6901 | tcaccctaat caagtttttt ggggtcgagg tgccgtaaag cactaaatcg gaaccctaaa |
| 6961 | gggagccccc gatttagagc ttgacgggga aagccggcga acgtggcgag gaaggaaggg |
| 7021 | aagaaagcga aaggagcggg cgctagggcg ctggcaagtg tagcggtcac gctgcgcgta |
| 7081 | accaccacac ccgccgcgct taatgcgccg ctacagggcg cgtcgcgcca ttcgccattc |
| 7141 | aggctgcgca actgttggga agggcgatcg gtgcgggcct cttcgctatt acgccagctg |
| 7201 | gcgaaagggg gatgtgctgc aaggcgatta agttgggtaa cgccagggtt ttcccagtca |
| 7261 | cgacgttgta aaacgacggc cagtgaattg taatacgact cactataggg cgaattcgag |
| 7321 | ctcggtaccc ctagagtcct gtattagagg tcacgtgagt gttttgcgac attttgcgac |
| 7381 | accatgtggt cacgctgggt atttaagccc gagtgagcac gcagggtctc cattttgaag |
| 7441 | cgggaggttt gaacgcgcag ccgcc |

### Methods of Treatment

The present disclosure provides methods for the intracranial delivery of therapeutic proteins to subjects in need thereof. In some embodiments, the therapeutic enzyme is β-hexosaminidase. In some embodiments, the therapeutic enzyme is acid β-galactosidase. In some embodiments, the enzyme is administered as a replacement for lost or diminished endogenous proteins such as β-hexosaminidase and/or acid β-galactosidase. In some embodiments, the enzyme is administered as a prophylactic measure in subjects predisposed to an enzyme deficiency.

Direct infusion of AAV vectors into the human brain parenchyma is an effective means to treat LSDs. However, the scale of the human brain represents an enormous challenge. It is estimated that that between 200-500 injections may be necessary to achieve the exceptional effects obtained in cat and dog models of LSDs (See FIG. 2). The choice of targets in the human brain should be guided by their effectiveness to provide vector-encoded enzymes throughout the CNS.

Distribution of lysosomal enzymes in the brain from vector-transduced cells occurs by diffusion in the brain parenchyma, but more importantly they are also transported over long distances via retrograde axonal transport to structures that send afferent connections to vector-transduced areas. There is also evidence suggesting that these enzymes may be distributed via anterograde transport. Distribution via the CSF flow in the perivascular space of Virchow-Robin also appears to contribute to widespread distribution of lysosomal enzymes in the brain. These properties of lysosomal enzymes can be explored/exploited to achieve global distribution of lysosomal enzymes throughout the brain.

The striatum has been the target of choice for AAV-mediated gene delivery to the brain in different LSD models. However the rationale for this choice of target for AAV-mediated gene delivery pre-dates the discovery of axonal transport as a means for distribution of lysosomal enzymes in the brain. In this regard the thalamus is an appealing target for genetic modification for widespread distribution of lysosomal enzymes throughout the mammalian brain because it receives afferent input from many structures throughout the CNS before sending the information to the cerebral cortex, from which it also receives reciprocal input. Therefore, the thalamus can be viewed as the central node in a ‘built-in’ network for widespread distribution of lysosomal enzymes throughout the CNS via axonal retrograde transport. Studies on AAV-mediated gene delivery of mouse βgal to the thalamus of adult GM1-gangliosidosis mice have shown distribution of enzyme throughout the injected hemisphere, and also in the brain stem, eye and spinal cord. Bilateral thalamic injections of AAV-βgal vector in adult GM1-gangliosidosis mice result in complete elimination of GM1-ganglioside storage throughout the brain.

AAV vector technology can be used to achieve efficient genetic modification of the thalamus in humans to create an enzyme producing ‘central node’ capable of distributing functional enzyme throughout the entire brain, and thus alter the course of disease progression in Tay-Sachs and Sandhoff diseases. This strategy should be applicable to many, if not all other LSDs with neurological involvement where the therapeutic protein can be secreted from genetically modified cells and taken up by diseased cells.

In some embodiments, the present disclosure provides methods for the enzyme replacement in a subject in need thereof. In some embodiments, the present disclosure provides methods for treating an enzyme deficiency. In some embodiments, the deficiency comprises a lysosomal storage disorder. In some embodiments, the disorder comprises Tay-Sachs Disease, Sandhoff Disease, or GM1-gangliosidosis. In some embodiments, the method comprises administering an AAV expression construct to a subject predisposed to having a lysosomal storage disorder. In some embodiments the subject is predisposed to having Tay-Sachs Disease, Sandhoff Disease, or GM1-gangliosidosis. In such embodiments, the method is directed to reducing the likelihood of onset of or severity of the disorder.

The specific method of treatment will vary depending on the embodiment. In the context of treating Tay-Sachs disease or Sandhoff Disease, the method comprises administering to a subject in need thereof an effective amount of a composition comprising a first expression construct and a second expression construct, wherein the first construct expresses the β-N-acetylhexosaminidase β subunit, and the second construct expresses the β-N-acetylhexosaminidase α subunit. Likewise, in the context of reducing the likelihood of onset of or severity of Tay-Sachs disease or Sandhoff Disease, the method comprises administering to the subject AAV expression vectors encoding the β-N-acetylhexosaminidase α and β subunits. In some embodiments, the method comprises administering to a subject in need thereof a therapeutically effective amount of a composition comprising a β-N-acetylhexosaminidase expression construct, wherein a single construct encodes both the α and β subunits of β-N-acetylhexosaminidase.

In the context of treating GM1-gangliosidosis, the method comprises administering to a subject in need thereof a composition comprising an expression construct encoding acid β-galactosidase. Likewise, in the context of reducing the likelihood of onset of or severity of GM1-gangliosidosis, the method comprises administering to the subject an AAV expression vector encoding acid β-galactosidase.

In some embodiments, the present disclosure provides methods directed to achieving widespread expression of β-N-acetylhexosaminidase or acid β-galactosidase in the brain of a subject in need thereof. In these contexts, the method comprises administering an AAV expression vector encoding the appropriate enzyme.

### Administration of AAV Compositions

The present disclosure provides methods for intracranial administration of AAV expression constructs to subjects in need thereof. In some embodiments, the methods include administering AAV constructs to the brain of a subject. In some embodiments, the method comprises administering the AAV composition to one or more areas of the brain selected from the thalamus, striatum, deep cerebellar nuclei, ventral tegmental area, and lateral ventricles. In some embodiments, the method comprises administering the composition unilaterally or bilaterally to the brain of the subject.

Any suitable means may be used to deliver AAV compositions to the brain loci of choice. The techniques discussed herein should be construed as exemplary and are intended to be limiting.

Exemplary methods for intracranial administration of AAV constructs include, in some embodiments, opening the cranium of the subject to gain access to the brain. In some embodiments, such methods include but are not are not limited to administering appropriate anesthesia to the subject, performing incisions at predetermined locations, creating burr holes in the skull at appropriate stereotaxic coordinates, and inserting a suitable instrument for the delivery of AAV compositions. In some embodiments, delivery devices include but are not limited to needles, cannulae, and catheters. Selection of the delivery device may depend on various factors including but not limited to the size and depth of the targeted loci, and the volume of AAV composition to be administered. In some embodiments, delivery of the AAV composition may be controlled by an external pump of appropriate design to allow the use to control the rate and duration of infusion. In some embodiments, upon completion of infusion, cranial incisions are closed using surgical staples or colloidin.

### Evaluation of Subjects Administered AVV Compositions

In some embodiments, the present disclosure provides methods for evaluating a subject who has been administered an AAV expression construct. In some embodiments, the subject is evaluated prior to administration, during administration, and after administration. In some embodiments, the AAV expression construct encodes β-N-acetylhexosaminidase or acid β-galactosidase. In some embodiments, multiple administrations are performed, as needed, to achieve or maintain the desired effect. For example, in some embodiments, the desired effect may be treatment of a disorder, reducing the likelihood of onset of a disorder, or reducing the severity of a disorder.

In some embodiments, evaluation of the subject comprises assessment of symptoms associated with a lysosomal storage disorder. In some embodiments, the disorder comprises Tay-Sachs Disease, Sandhoff Disease, or GM1-gangliosidosis. Such symptoms include but are not limited to neurological deficits such as motor and visual deficits. [Inventor: Please provide a list of symptoms/evaluations that might be performed for a human subject.]

In some embodiments, evaluation of the subject comprises a biochemical assessment of enzyme level and/or enzyme activity. In some embodiments, the enzyme comprises β-N-acetylhexosaminidase. In other embodiments, the enzyme comprises acid β-galactosidase. Biochemical assessment of enzyme level and/or enzyme activity may be accomplished by methods known in the art including but not limited to measuring enzyme level and/or activity in the serum of the subject.

### Pharmaceutical Compositions

Pharmaceutical compositions suitable for injectable use can include sterile aqueous solutions (where the components are water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). Typically, a composition for parenteral administration is sterile and fluid to the extent that easy syringability exists. Such compositions are generally stable under the conditions of manufacture and storage and are preserved against the contaminating action of microorganisms such as bacteria and fungi.

The pharmaceutical compositions can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

### Dosage

Dosage may be determined in accordance with the methods described herein using pharmaceutical preparations of AAV expression constructs. The dosage ranges described herein are to be construed as exemplary and are intended to be limiting.

Dosage, toxicity, and therapeutic efficacy may be determined by standard pharmaceutical procedures in cell cultures or experimental animals, for example, to determine the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. In some embodiments, the dosage of such compounds lies within a range of circulating concentrations that include the ED₅₀ with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some embodiments, an effective amount of a composition sufficient for achieving a therapeutic or prophylactic effect, ranges from about 0.000001 mg per kilogram body weight per administration to about 10,000 mg per kilogram body weight per administration. Suitably, the dosage ranges are from about 0.0001 mg per kilogram body weight per administration to about 100 mg per kilogram body weight per administration. Administration can be provided as an initial dose, followed by one or more additional doses. Additional doses can be provided a day, two days, three days, a week, two weeks, three weeks, one, two, three, six or twelve months after an initial dose. In some embodiments, an additional dose is administered after an evaluation of the subject's response to prior administrations.

The skilled artisan will appreciate that certain factors may influence the dosage and timing required to effectively treat a subject, including but not limited to, the severity of the disease or disorder, previous treatments, the general health and/or age of the subject, and other diseases present. In addition to these factors the dosage of an AAV vector infused into particular structures may be limited by the volume that can be infused without causing toxicity/damage that may lead to further neurological deterioration.

Considerations:
1. Volume that can be safely infused into particular target structures without causing damage, which is also a function of the infusion technology that is employed.
2. Maximum possible vector stock concentration that is devoid of virion particle aggregation (˜1-2E13 gc/ml).
3. AAV vector dose should lead to overexpression of the lysosomal enzymes in target structures sufficient to supply the CNS with therapeutic levels in the absence of toxicity to the target structure(s). This may be recognized by the onset of symptoms atypical for the disease being treated.
4. AAV vector dose should be such that it significantly slows, or stops disease progression using clinical parameters and imaging approaches such as Magnetic resonance imaging (MRI) and Magnetic resonance spectroscopy (MRS).
5. Reduction in GM2-ganglioside levels in CSF should be measurable in patients receiving AAV vector infusions.
6. Dose range that may be useful in humans would be 1E11-1E14 gc/brain.

In an exemplary determination, the minimum effective dose of an AAV expression construct may be defined to be that which results in greater than 75% decrease in ganglioside content in the brain or cerebellum after bilateral thalamic or cerebellar (deep cerebellar nuclei, DCN) injections, respectively. In an exemplary determination, the AAV expression construct may comprise expression constructs encoding the β-hexosaminidase α and β subunits. Dosage and concentration may be expressed in terms of genome copies (gc) or viral genomes (vg) per kilogram body weight or per milliliter.

### EXAMPLES

The methods of the present disclosure are further described by the following examples. These examples are to be construed as illustrative and are not intended to be in any way limiting.

### Materials and Methods

### Animal Subjects

GM1 gangliosidosis (GM1) mice are described in Hahn et al., 6 Hum. Mol. Genet. 205-211 (1997). Tay-Sachs disease (TS) mice are described in Yamanaka et al., 91 Proc. Natl. Acad. Sci. U.S.A. 9975-9979 (1994). Sandhoff disease (SD) mice are described in Sango et al., 11 Nat. Genet. 170-176 (1995). GM1 gangliosidosis (GM1) cats are described in Baker, et al., 174 Science 838-839 (1971). GM2 gangliosidosis (GM2) cats are described in Cork, et al., 196 Science 1014-1017 (1977).

### AAV Vector Design and Preparation

General—

AAV vectors were produced by co-transfection of 293T cells by calcium phosphate precipitation of vector plasmid (AAV-CBAGFP-W), a mini-adenovirus helper plasmid pFΔ6, and AAV1 helper plasmid pXR1 (Rabinowitz et al., 2002); or AAV2 helper plasmid pH22 (Hauck and Xiao, 2003); or AAVrh.8 helper plasmid pAR8 constructed using DNAWorks 2.4 software (http://molbio.info.nih.gov/dnaworks/) to generate primers for PCR-based synthesis (Hoover and Lubkowski, 2002) of the AAVrh.8 capsid gene (Gao et al., 2002). The amplified PCR product was cloned into pXR-1, generating pAR-8. Integrity of the AAVrh.8 Cap insert was verified by sequencing. Sixty hours post-transfection cells were harvested and the AAV vectors purified using a discontinuous iodixanol gradient followed by anion exchange chromatography using HiTrap Q columns in an A″ KTAprime liquid chromatography system (Amersham Biosciences AB, Uppsala, Sweden), essentially as described (Zolotukhin et al., 2002). The vector stocks were concentrated and the buffer exchanged to phosphate-buffered saline (PBS) in Centricon-P20 concentrators (Biomax 100K, Millipore, Bedford, Mass., USA), as described (Zolotukhin et al., 2002). AAV vector titers (genome copies/ml or g.c./ml) were determined as described (Veldwijk et al., 2002) by real-time quantitative PCR in a Light Cycler (Roche, Indianapolis, Ind., USA) using the following primers and probes (TIB Molbiol LLC, Adelphia, N.J., USA) specific for the bovine growth hormone polyadenylation signal present in the vector: BGHpolAF2: CCTCGACTGTGCCTTCTAG; BGHpolAR2: CCCCAGAATAGAATGACACCTA; hybridization probe BGHpolA fluorescein: GCCACTCCCACTGTCCTTTCCTAA-FL; hybridization probe BGHpolA LC Red640: LC Red640-AAAATGAGGAAATTGCATCGCATTGTCT.

Recombinant AAV viruses were produced by triple plasmid cotransfection of HEK 293 cells by using pAAVSP70 harboring the expression cassettes, an adenovirus helper plasmid, and a chimeric packaging construct expressing the AAV2 rep gene and either the AAV2 or AAV1 cap genes. rAAV2_{—}2 viruses were purified by affinity column chromatography (29) (produced at the University of Pennsylvania Vector Core Facility, Philadelphia). rAAV2_{—}1 viruses were purified by ion-exchange chromatography (produced at Genzyme Corp.). DNase-resistant viral genome copies (drps) of the AAV vectors were determined by using a real-time TaqMan PCR assay (ABI Prism 7700; Applied Biosystems, Foster City, Calif.) with primers specific for theBGHpAsequence.

β-Galactosidase Expression Constructs—

The design and production of AAV2/1-CBA-βgal vector carrying the mouse lysosomal acid β-galactosidase (βgal) cDNA under the CBA promoter, which is comprised of the CMV immediate-early enhancer fused to the chicken beta-actin promoter, was described previously (Broekman et al, 2007). The plasmid pAAV-ApoE4hAAT-βgal-W was constructed by replacing the CBA promoter in the plasmid pAAV-CBA-βgal-W with the hybrid ApoE4/hAAT liver specific promoter (human alpha-1 antitrypsin promoter fused to 4 copies of the apolipoprotein A enhancer (Schuettrumpf et al, 2005). The AAV2/rh.8-ApoE4hAAT-βgal vector was prepared as described (Broekman et al, 2006). All vectors used in this study carry the woodchuck hepatitis virus post-transcriptional regulatory element (WPRE).

Hexosaminidase Expression Constructs—

The AAV2 plasmid vector used for the production of rAAV2/2α, rAAV2/1α, rAAV2/2β, and rAAV2/1β was generated by subcloning the expression cassettes encoding human β-hexosaminidase α and β subunits into pAAVSP70, a derivative of pAV1 (28). To generate the expression cassettes hexA and hexB, cDNAs were synthesized by RT-PCR (Stratagene) by using total RNA isolated from human liver as a template and cloned into the plasmid pcDNA3 (Invitrogen). The woodchuck hepatitis virus posttranscriptional regulatory element (WPRE) was amplified by PCR from viral genomic DNA (American Type Culture Collection, Middlesex, U.K.) with primers and cloned downstream of the hexa and hexβ fusion cDNAs. The bovine growth hormone polyadenylation signal sequence (BGHpA) was that of plasmid pcDNA3. The composite promoter CAG was cut from plasmid pDRIVE-CAG (InvivoGen, San Diego) and cloned into plasmid pAAVSP70 upstream of the transcriptional cassettes.

AAV vectors used for production of AAV2/rh8 stocks encoding human or feline HexA α- or β-subunits were constructed by PCR amplification using the following templates: Human a-subunit: IMAGE ID: 3353424 MGC-14125, Genbank: BC018927; Human β-subunit: IMAGE clone IMAGE ID: 2967035, GenBank: BC017378; Feline a-subunit: cDNA was cloned using RNA isolated from cat tissues; Feline β-subunit: plasmid provided by Dr. Douglas R. Martin. The PCR amplified cDNAs were cloned into the plasmid AAV2/1-CBA-βgal replacing the βgal cDNA.

### Delivery of AAV Vector to the Mouse Brain

Six to eight week-old GM1 mice were anesthetized by intraperitoneal injection of ketamine (125 mg/kg) and xylazine (12.5 mg/kg) in 0.9% saline, and placed in a small animal stereotaxic frame (Stoelting, Wood Dale, Ill.). An incision was made over the skull, the periosteum removed and a burr hole was made at the appropriate stereotaxic coordinates using a high-speed drill (Dremel, Racine, Wis.). The noncompliant infusion system used in these experiments for delivery of AAV vector was assembled using a Harvard 22 syringe pump (Harvard Apparatus, Holliston, Mass.) to drive a gas-tight Hamilton Syringe (Hamilton, Reno, Nev.) attached to a 33-gauge steel needle (Hamilton) via 1/16″×0.020″ ID PEEK tubing (Alltech, Deerfield, Ill.) and Luer adapters (Amersham Biosciences). First the syringe and tubing were filled with sterile mineral oil and then vector stock was withdrawn into the needle and line. The needle assembly (needle+Luer adapters) was fixed to the arm of the stereotaxic frame. AAV2/1-CBA-βgal vector was infused (1 μl at 0.2 μl/min) into the left thalamus (AP −2.0 mm, ML −1.5 mm relative to bregma, and DV −2.5 mm from the brain surface). In subsequent therapeutic efficacy experiments the AAV2/1-CBA-βgal vector was infused (0.2 μl/min) bilaterally into the thalamus at two depths (AP −2.0 mm, ML +/−1.5 mm relative to bregma; and −3.5 and −2.5 mm from the brain surface; 1 μl per depth) (total dose per mouse=4.8×10¹⁰ gc), or bilaterally into the thalamus (as above) and deep cerebellar nuclei (AP: −6.3 mm; ML: +/−1.5 mm; DV −2.0 mm; 1 μl per side) (total dose per mouse=7.2×10¹⁰ gc). In the PBS control group, age matched GM1 mice received bilateral infusion of PBS into the thalamus and deep cerebellar nuclei (same infusion rate and volume as above). The needle was left in place for 2.5 min after the injection was finished and then retracted halfway and left in place for an additional 2.5 min before complete withdrawal. The incision was closed with surgical staples, or colloidin, and the animal was allowed to recover completely before being returned to the holding room.

### Delivery of AAV Vector to the Cat Brain

Cats were tranquilized with ketamine (10-20 mg/kg), Domitor (0.1-0.2 ml) and glycopyrrolate (0.2 mg/ml) and maintained by tracheal intubation and inhalation anesthesia with isoflurane (1-3% in oxygen). Injections were performed with a Horsley-Clark stereotaxic apparatus (David Kopf Instruments) in the surgical suite of the Scott-Ritchey Research Center. Four to six-week old GM2 gangliosidosis cats were injected bilaterally into the thalamus and deep cerebellar nuclei (dcn) using the following coordinates relative to bregma: thalamus, AP −7.5 mm, ML ±5.0 mm, DV −16.5 mm; deep cerebellar nuclei, AP −29.5 mm, ML ±5.0 mm, DV −13.5 mm. After injection of 10 μl at the initial DV coordinate, the needle was raised in 1.0 mm steps and 10 μl was injected at each ascending step. A total of 60 μl and 20 μl of AAV vector formulation was injected per thalamus and dcn, respectively; the injection rate was 2.5 μl/min. Butorphanol was given for post-operative analgesia. Injection, surgery, and recovery occurred on a water-filled heat blanket to maintain body temperature.

### Neurological Testing

Tremor and bradykinesia were evaluated by inspection of mutant, compared with wild-type or heterozygous mice, after removal from their cages to a flat surface. Horizontal bar and inverted screen tests were used between 0900 and 1800 hours to score combined motor coordination, balance, and limb strength, which vary with time and in response to interventions (5). In the inverted screen test, the mouse was positioned in the center of a metal mesh and slowly inverted. Latency of falling from the screen over a padded surface, as well as the number of times the hind paws released and grasped the mesh, was recorded within 2 min (5).

Rotarod Test—

A rotarod apparatus, consisting of a knurled dowel fixed 10 cm above bedding was used to measure motor coordination and balance as previously described. After a 3-day pretrial training period, mice were assessed for motor behavior at 1, 2.5, 4, and 6 months post injection. Mice were placed on the rotating dowel at 20 rpm, indicating the start time for the trial. A 30-second interval was allowed between the two trials at the given speed. The maximum time allowed on the bar for each trial was 60 seconds. The trial was terminated when the mouse fell off the bar or at 60 seconds.

Open-Field Test—

Locomotor activity and rearing events in the mice were assessed using the SmartFrame Cage Rack System (Kinder Scientific, San Diego, Calif.). Infrared beams along the frame of the system track mouse movement in the cage with respect to location, distance, and rearing capabilities. Mice were placed in the center of the open-field apparatus and behavior was measured for 15 minutes. The data was analyzed using the MotorMonitor software (Kinder Scientific, San Diego, Calif.). Locomotor activity was measured as the distance traveled (in inches) and rearing events were measured as the number of times the mouse stood on its hind legs. Comparisons were narrowed to the first 5 minutes when significant differences between untreated GM1 and HZ mice were observed.

### Visual Evoked Potentials

Visual evoked potentials (VEPs) were recorded in AAV-treated GM1 mice (AAVT and AAV-TC groups; n=3 for each group) at 9-10 months of age. The VEPs were also recorded from untreated heterozygote (n=2) and GM1 (n=4) mice at 10 months and 7-8 months of age, respectively, according to previously described methods. Briefly, the mice were dark-adapted overnight and then anesthetized. The left pupil was dilated and responses were elicited with 10-μs full-field flashes of white light presented every second at 3.4 log ft.L. VEPs were monitored with subdermal electrodes in the scalp over the visual cortex as the positive electrode and over the frontal cortex as the reference. The responses were collected as previously described. The consecutive waveforms were averaged (n=100) after suppressing the heart-beat artifact with an adjustable low-pass digital filter (cut-off at 50-70 Hz) and rejecting waveforms containing movement artifacts.

### Statistical Analyses.

Unless otherwise indicated, data are expressed as means with SD; comparisons between groups were evaluated by the Mann-Whitney test. To avoid bias due to differences in survival between treated and untreated mice, the effect of gene therapy on hind-limb movement was considered only during the first 120 days of life. For each observation, the frequency of hind-limb movement was calculated, and a linear model fitted to the observations from each animal to detect any trends in that frequency with time. The linear model was weighted by observation time to account for the increased confidence in some estimates of the frequency. The linear coefficients from the models then were compared graphically; proportions with increasing or decreasing trends were compared by Fisher's exact test.

### Tissue Processing and Staining

Mice were fixed by intracardiac perfusion with paraformaldehyde. Sections (45 μm) were exposed to rat anti-mouse CD68 (Serotec, Oxford,) and G. simplicifolia isolectin B4 (biotinylated, ″-D-galactosyl-specific, Vector Laboratories, Peterborough, U.K.). Staining was based on the avidin-biotin peroxidase technique (31). Biotinylated rabbit anti-rat IgG secondary antibody (Vector Laboratories) and isolectin were detected by using Vectastain (Vector Laboratories) and developed with 3,3′-diaminobenzidine with Cresyl violet as counter stain. B-hexosaminidase activity was detected with naphthol AS-BI N-acetyl-β-glucosaminide (Sigma, Poole Dorset, U.K.) (32). PAS staining (Sigma) was followed by counterstaining with haematoxylin. Sections were mounted in dibutyl phthalate xylene (BDH).

β-Galactosidase Activity.

Mice were sacrificed by CO₂ asphyxiation at 1 or 4 months post-injection or at the humane endpoint defined by >20% loss in maximal body weight. The brains were harvested at 1 and 4 months post injection and at the humane endpoint. The left hemisphere of the brain was embedded in tissue freezing medium (Triangle Biomedical Sciences, Durham, N.C.) and rapidly frozen in a 2-methylbutane/dry-ice bath. Consecutive 20-μm thick coronal cryosections were prepared and stored at −80° C. One series of frozen sections representing the entire brain from AAV-treated GM1 mouse, or control non-injected GM1 mice was fixed for 10 min in 0.25% glutaraldehyde in phosphate buffered saline (PBS) at room temperature followed by two washes in PBS. Sections were stained for βgal using X-gal solution [5 mM K₄Fe(CN)₆, 5 mM K₃Fe(CN)₆, 2 mM MgCl₂, 1 mg/ml 5-bromo-4-chloro-3-indolyl-D-galactosidase(X-gal) in PBS, pH 5.0 using methods well known in the art.

Hexosaminidase Activity.

Mice were sacrificed by CO2 inhalation. The brain, cerebellum and spinal cord from each mouse was frozen in liquid isopentane maintained in equilibrium with solid CO2 and cut in 10-20 μm coronal (brain), sagittal (cerebellum) or transverse (spinal cord) sections. Enzyme distribution and vector distribution. One group of sections representing the entire brain, cerebellum, and spinal cord was stained for β-hexosaminidase as previously described. Distribution of AAV vector-transduced cells in the brain, cerebellum and spinal cord was assessed by non-radioactive in situ hybridization using an anti-sense riboprobe for WPRE, and standard techniques.

Tissue extracts in 0.01 M phosphate citrate buffer (pH 4.4) were assayed fluorimetrically for β-hexosaminidases with 4-methylumbelliferyl-β-N-acetylglucosaminide as substrate (Sigma). HEXA was calculated as the difference between total β-hexosaminidase (before) and HEXB activity (after) heat inactivation (12). Fluorescence was determined in a PerkinElmer LS30 fluorimeter. Protein was quantified by the Pierce protein assay (MicroBCA Reagent).

Electron Microscopy.

Tissues were fixed by perfuse-fixation with 50 ml intracardiac PBS (pH 7.4) containing 0.05% sodium nitrite was followed by 100 ml of 1% paraformaldehyde, 3% glutaraldehyde in 0.1M Pipes (pH 7.4) with 4 h after fixation at 4° C.; after several washes in buffer, tissues were treated with 1% osmiumferricyanide for 1 h at 4° C. and stained with 1% uranyl acetate and lead citrate. Thin sections were examined after dehydration and embedding.

Neuropathology and Inflammation.

Histology of the cortex, white matter, basal ganglia, brain stem, cerebellum, and spinal cord were examined using hematoxylin and eosin staining Tissue was examined for evidence of immunologically mediated insults. Small blood vessels were evaluated for vasculitis, neuronophagia, and micro-infarcts. Gray and white matter were evaluated for hypercellularity. Tissues were also evaluated for gliosis (GFAP immunostain; Serotec, Raleigh, N.C.), microglia activation (MHC II or Iba1 immunostain) and inflammatory infiltrates (CD68, CD4, CD8; Serotec).

Filipin Staining.

Tissue sections (20 μm) were fixed for 10 min in 4% paraformaldehyde in PBS at room temperature, followed by 3×5 min washes in PBS and 10 min incubation in 1.5% glycine in PBS. After 3×5 min washes in PBS, sections were incubated for 1 h at room temperature with Filipin (0.05 mg/ml; Sigma, St Louis, Mo.) and TOPRO-3 (1:1,000; Molecular Probes, Eugene, Oreg.) in PBS. Sections were washed (as above) and coverslipped with fluorescent mounting media (DakoCytomation).

For glycosphingolipid analysis, lyophilized aqueous tissue homogenates were extracted with chloroform:methanol (2:1), dried under nitrogen and after redissolving in solvent, extracts equivalent to 250-500# g of dried tissue were separated alongside pure standards by high-performance thin-layer chromatography (silica gel 60, Merck) in chloroform:methanol: 0.22% CaCl₂ (60:35:8, vol″vol). Dried plates were sprayed with orcinol:sulfuric acid and baked at #90° C. for 15 min. The intensity of individually resolved species was quantified by densitometry by using NIH IMAGEJ software employing galactocerebroside present in each extract to correct for loading differences.

### Biochemical Quantification of β-Hexosaminidase Activity and GM2-Ganglioside Levels

Tissue samples were homogenized in water and small aliquots used to measure hexosaminidase enzymatic activity using the synthetic substrates 4-methylumbelliferyl-N-acetylglucosamine (4-MUG; Sigma), and its sulfate derivative 4-methylumbelliferyl-N-acetylglucosamine-6-sulfate (4-MUGS; Toronto Research Chemicals) as previously described. Homogenates were further processed for lipid isolation.

Isolation and Purification of Brain and Fluid Lipids.

Total brain gangliosides, asialo-glycosphingolipids, neutral and acidic lipids were isolated and purified using methods well known in the art. The total content of ganglio-series gangliosides in brain tissues and in CSF were estimated using highly sensitive TLC immunostaining with anti-ganglioside antibodies. Qualitative and quantitative analysis of the individual ganglioside species were performed by high performance thin-layer chromatogram (HPTLC) as previously described. The density values for each lipid were fit to a standard curve and used to calculate individual lipid concentrations. Gas-liquid chromatography was used to measure the content of N-acetyl (NeuAc) and N-glycolyl (NeuGc) neuraminic acids.

### Separation of β-Hexosaminidase Isoforms

Separation of β-Hexosaminidase Isoforms by Column Chromatography.

Samples (cell pellets or tissue) were homogenized (glass homogenizer) in 1.5 ml of de-gassed 10 mM sodium phosphate buffer pH 6.0 containing 100 mM sodium chloride and 0.1% Triton X-100. Buffers, samples and homogenates were kept on ice during the entire procedure. The homogenates were centrifuged at 10,000 rpm at 4° C. for 10 minutes. Supernatants were then filtered through a small 0.2 μm filter unit and kept on ice until run on a 1 ml Resource™ Q column (GE Healthcare; code #17-1177-01). Samples were resolved in a linear gradient of 100-400 mM sodium chloride solution. Solutions were kept on ice while running through the column. Fractions (500 μl) were collected, including the wash through (HexB does not stick to the column and comes out in the first few fractions). Fractions were kept on ice until analyzed or frozen after aliquoting to avoid thawing and re-freezing. Fractions were assayed for enzymatic activity using 4-MUG and 4-MUGS.

### Example 1

### AAVrh.8-Mediated Gene Therapy in GM1 Mice

(i) Distribution of βgal in the Brain of GM1 Mice Following Unilateral Intrathalamic Infusion of AAVrh.8-βgal.

One μl of an AAVrh.8-βgal (6.13×10¹³ gc/ml) was injected into the left thalamus of 6-8 week-old GM1-gangliosidosis mice. βgal expression and distribution throughout the brain was evaluated at 4 weeks post-injection by X-gal staining (FIG. 3). Scale bars=1 mm. βgal expression was evident throughout the injected brain hemisphere (FIG. 3).
(ii) Distribution of βGal Outside the Cerebellum of GM1 Mice Following Unilateral Intrathalamic Infusion of AAVrh.8-βGal.

One μl of an AAVrh.8-βgal (6.13×10¹³ gc/ml) was injected into the left thalamus of 6-8 week-old GM1-gangliosidosis mice. βgal expression and distribution throughout the brain was evaluated at 4 weeks post-injection by X-gal staining (FIG. 4). Scale bars in FIG. 4A, B=1 mm; Magnifications: C, D, F-200×; E-40×.

Cerebellum in uninjected (FIG. 4A) and injected (FIG. 4B) GM1-gangliosidosis mice. Arrowhead in (FIG. 4B indicates the inferior colliculus, and arrow indicates the brain stem. βgal was absent in the left eye (FIG. 4C) but it was present in the ganglion cell layer (GCL) in the right eye (FIG. 4D). In the spinal cord (FIGS. 4E, F) β-gal activity was found in the ascending sensorimotor pathway (FIG. 4E, arrow) and in cells in the dorsal horn (FIG. 4F, arrow).
(iii) Lysosomal Storage in the Brain of GM1 Mice Following Unilateral Intrathalamic Infusion of AAVrh.8-βgal.

Unesterified cholesterol storage in the brains of AAVrh.8-treated (FIGS. 5A-F) and untreated (FIG. 5G-I) GM1-gangliosidosis mice was assessed by Filipin staining (blue) at two weeks post-infusion. In the ipsilateral hemisphere of AAVrh.8-treated mice there was considerable reduction in lysosomal storage (FIG. 5A-C), while in the contralateral hemisphere (FIG. 5D-F) storage levels appeared comparable to those found in control untreated mice (FIG. 5G-I). Nuclei were counterstained with TO-PRO3 (red). Scale bar=200 μm.
(iv) Biochemical Quantification of GM1-Ganglioside in the Brain of GM1 Mice Following Bilateral Intrathalamic Infusion of AAVrh.8-βGal.

Levels of GM1-ganglioside in the cerebral cortex were quantified at 4 months post-AAV treatment. (FIG. 6A) HPTLC of cortical gangliosides; (FIG. 6B). Quantitative analysis of total gangliosides and GM1-ganglioside content. AAV-treated animals show a marked reduction in GM1-gangliosides compared to control animals, to levels similar to that of heterozygous littermates (FIG. 6).
(v) Effects of Infusion of AAVrh.8-βgal on Motor Performance in GM1 Mice.

The effects of AAV-mediated βgal expression on the motor performance of GM1 mice was evaluated using Rotarod and Open field testing (FIG. 7). Mice were infused with AAV-βgal expression constructs bilaterally in the thalamus (AAV-T group) or bilaterally in the thalamus and deep cerebellar nuclei (AAV-TC group). Rotarod testing was performed prior to injection (0 months), and then at 1, 2.5, 4, and 6 months post-injection in AAV-T GM1 mice (•), AAV-TC GM1 (X), untreated GM1 (▴), and heterozygous (HZ) mice (♦) (FIG. 7A). Open-field testing measured locomotor (FIG. 7B) and rearing activity (FIG. 7C) at 2.5 (2.5M) and 4 (4M) months post-injection in HZ (white bars), untreated GM1 (black bars), AAV-T GM1 (light gray bars), and AAV-TC GM1 (dark gray bars) mice. Group sizes: n=20-24 for 0 and 1 month time points; n=14-18 for 2.5 and 4 month time points; n=10-12 for 6 month time point. Graphs represent the mean for each group at the specified time point. Error bars correspond to 1 SEM. *p<0.05 in paired Student's t-test.

Rotarod testing of all animals prior to intracranial injection of AAV vector showed comparable performance for all groups of mice (FIG. 7A). Mice were subsequently tested at 1, 2.5, 4, and 6 months post-injection. The performance of either group of AAV-treated GM1 mice (AAV-T and AAV-TC) declined over time, and was indistinguishable from that of untreated GM1 mice (FIG. 7A). The performance of heterozygous littermates remained essentially stable for the duration of the experiment.

At 2.5 months post-injection the locomotor activity of both groups of AAV-treated GM1 mice was greater than untreated GM1 controls (FIG. 7B, 2.5M—black bar), but statistical significance (p<0.05) was only achieved in AAV-T GM1 mice (FIG. 7B, 2.5M—light gray bar). By 4 months post-injection the locomotor activity of AAV-treated GM1 mice declined and was indistinguishable from untreated GM1 control mice (FIG. 7B, 4M). The locomotor activity of untreated GM1 mice increased between the two time points (FIG. 7B, black bars, p<0.01), while that of HZ controls decreased (FIG. 7B, white bars, p<0.05). Although the number of rearing events in AAV-treated GM1 mice (FIG. 7C, gray bars) was consistently higher than in untreated GM1 mice controls (FIG. 7C, black bars), this difference was significant only in AAV-T GM1 mice at 2.5 months post-injection (p<0.05).
(vi) Effects of Bilateral Intrathalamic Infusion of AAVrh.8-βGal on Visual Function in GM1 Mice.

The effects of AAV-mediated βgal expression on the visual function of GM1 mice was evaluated by measuring visual evoked potentials (FIG. 8). Mice were infused with AAV-βgal expression constructs bilaterally in the thalamus (AAV-T group) or bilaterally in the thalamus and deep cerebellar nuclei (AAV-TC group). Potentials were measured in (FIG. 8A) wild type, (FIG. 8B) HZ, (FIG. 8C) untreated GM1, (FIG. 8D) AAV-T GM1, and (FIG. 8E) AAV-TC GM1 mice. Group sizes are indicated on the graphs. (FIG. 8C-E) Gray lines show the results for each mouse in the group. Black lines represent the group average.

GM1 mice older than 6 months display visual abnormalities characterized by normal electroretinograms but subnormal visually evoked potentials (VEP). VEPs were analyzed in AAV-treated GM1 mice at 10-11 months of age, and age matched untreated control HZ mice (FIG. 8). Untreated GM1 mice were analyzed at 7-8 months of age, and presented subnormal VEPs (FIG. 8C) compared to wild type (FIG. 8A) and HZ (FIG. 8B) mice. AAV-treated GM1 mice showed some response to the visual stimulus (FIGS. 8D, E), albeit with considerable variability among animals within each group (gray lines in FIGS. 8D, E represent the VEP of each individual animal in the group). The VEP data also show, on average, normal negative peak implicit time (50-75 msec) for AAV-treated mice. These data suggest that AAV-treated GM1 mice retained some degree of visual functionality past 6 months of age. Histological analysis of the eye at the humane endpoint (untreated GM1 and AAV-T GM1 mice), or 1 year of age (heterozygote mice, and AAV-TC GM1 mice) showed evidence of some βgal activity in the retinal ganglion cell layer (GCL) in both groups of AAV-injected GM1 mice compared to no detectable activity in the retinas of untreated GM1 mice (not shown).

### Example 2

### AAV-Mediated Gene Therapy in GM1 Cats

(i) Widespread Distribution of βgal Activity in the Feline GM1 Brain Following Intracerebroventricular (ICV) Delivery of AAV-βGal Expression Constructs.

For intracerebroventricular (ICV) delivery of βgal expression constructs, three AAV serotypes were tested: AAV2/rh8, AAV2/1, and AAV2/9. In terms of βgal distribution and activity levels throughout the brain, the serotype ranking was AAV2/rh8>AAV2/1>AAV2/9.

Table 2 summarizes the results of βgal distribution as evaluated by X-gal staining Ultrasound-guided injection of the left lateral ventricle was performed to deliver ˜0.2 ml of each vector serotype. The vector backbone for each serotype was identical: AAV2-CBA-fBgal-WPRE. The dose listed is the total number of genome equivalents (g.e.). The age at treatment (Tx) is given in weeks. The elapsed time between treatment and evaluation (duration) is given in days. Coronal brain sections were stained with Xgal (pH 4.7) and the staining intensity (β-gal stain) rated on a scale of 1-10, where 1 is minimal staining and 10 is intense staining (equivalent to direct intraparenchymal injection, discussed below). βgal scoring is given for the brain as a whole. For each serotype, n=3 animals.

TABLE 2
β-gal activity in the feline GM1 brain after ICV
delivery of AAV-βgal expression constructs.

| GM1 | Tx Age | Duration | | | Bgal |
|---|---|---|---|---|---|
| Cat | (wks) | (d) | Serotype | Dose (g.e.) | stain |
| 8-1245 | 10.7 | 29 | 1 | 7.0E+12 | 1 |
| 8-1454 | 10.4 | 31 | 1 | 4.5E+12 | 1.5 |
| 9-1307 | 12.4 | 36 | 1 | 1.6E+13 | 2 |
| 8-1288 | 12.7 | 28 | rh8 | 4.2E+12 | 5 |
| 8-1290 | 13.4 | 28 | rh8 | 4.1E+12 | 4 |
| 8-1449 | 9.1 | 28 | rh8 | 3.3E+12 | 1 |
| 9-1410 | 8.0 | 29 | 9 | 4.4E+12 | 1 |
| 9-1418 | 8.7 | 30 | 9 | 4.7E+12 | <1 |
| 9-1425 | 9.9 | 30 | 9 | 4.8E+12 | <1 |

(ii) Distribution of βgal Activity in the Feline GM1 Brain and Spinal Cord Following Unilateral Intrathalamic Infusion of an AAV-βGal Expression Construct.

A GM1 cat was injected in the right thalamus with 1.85×10¹² g.e. of AAV2/rh8-CBA-fBgal-WPRE, in which a CBA promoter drives expression of a feline βgal cDNA. One month later, brain was collected, cryosectioned at 40 μm, and stained with the histochemical substrate Xgal (pH 4.7) to detect lysosoma βgal. To facilitate cryosectioning, large coronal blocks were halved prior to freezing, shown above as midline horizontal separations of some sections. βgal was detected throughout the entire injected cerebrum, 1.8 cm anterior and 0.6 cm posterior to the injection (Inj) site (FIG. 9). βgal activity ranged from 1.3-4.1 times normal (fold normal βgal) when quantitated with the fluorogenic substrate 4-methylumbelliferyl (4MU)-B-D-galactopyranoside. Xgal-stained control sections are shown from normal and GM1 brain, which expresses <5% normal βgal activity. Enzyme was evenly distributed throughout the injected cerebrum, with all sites anterior to and including the injection site expressing ˜4 times normal βgal activity. Though not injected, the ipsilateral cerebellum exhibited intense focal areas of enzymatic activity, likely to have resulted from neuron-mediated βgal transport from the injection site (or adjacent sites) to cerebellar nuclei. Overall, the ipsilateral cerebellum expressed 15.8% normal βgal activity (data not shown).

Vector delivery by ICV infusion also resulted in very high levels of βgal expression in the spinal cord of GM1 cats (FIG. 10). Using ultrasound guidance, a GM1 cat was injected into the left lateral ventricle with 4.2×10¹² g.e. of vector AAV2/rh8-CBA-fBgal-WPRE. One month later, brain was sectioned at 50 μm and stained for βgal activity with Xgal (pH 5.1). All spinal cord segments in the treated GM1 cat (GM1+AAV) exhibited normal or above normal levels of staining (FIG. 10). Spinal cord segments are as follows: rostral cervical (RC), mid-cervical (MC), cervical intumescence (CI), mid-thoracic (MT), thoracolumbar (TL), mid-lumbar (ML) and lumbar intumescence (LI). Untreated normal and GM1 spinal cord segments (both LI) are included as controls.
(iii) Quantification of βgal Activity in the Feline GM1 Brain 1 Month Post AAV Treatment.

Three GM1 cats were injected bilaterally into the thalamus and deep cerebellar nuclei with a total dose of 3×10¹² g.e. AAV2/rh8-CBA-fBgal-WPRE. βgal activity in the brain was assessed 1 month later by X-gal staining Brains were sectioned into 0.5 cm coronal blocks for cryoembedding, and blocks were sectioned at 50 um for homogenization and fluorogenic measurement of βgal activity. Distances in cm anterior (+) or posterior (−) to the injection site (Inj) are shown for cerebrum or cerebellum (Cblm). All data was normalized to blocks from normal cats. Untreated GM1 cats expressed <5% normal activity in all blocks. βgal activity was detected throughout the cerebrum and cerebellum at levels ranging from 38.3-278.5% normal (FIG. 11).
(iv) Long-Term Therapeutic Benefit of AAV-βGal Infusion in GM1 Cats.

After demonstrating dramatic restoration of enzymatic activity by histochemical and fluorogenic substrates, long-term therapeutic studies were initiated with AAV2/1 and AAV2/rh8 vectors. Results are summarized in Table 3. Age at AAV treatment is given in weeks. Time elapsed at the time of scoring is given in months. Clinical ratings were assigned according to the scale given Table 4.

Treatment of GM1 cats with AAV-mediated βgal expression resulted in suppression of clinical symptoms of GM1 gangliosidosis. The human endpoint for untreated GM1 cats is 7.7±0.8 months and is defined by the subject's inability to support weight on its forelimbs over two consecutive days or the loss of 20% of maximal body weight (scores 2 and 1, respectively, on the clinical rating scale given in Table 4). GM1 cats treated with AAV-βgal constructs scored 10 on the clinical rating scale as long as 11.4 months after treatment (subject 9-1356).

TABLE 3
Summary of long-term therapeutic
benefit in AAV-treated GM1 cats.

| | Sero- | Tx Age | Time Post- | Clinical rating | Clinical |
|---|---|---|---|---|---|
| Cat | type | (mo) | Tx (mo) | score (CRS) | description |
| 9-1356 | 1 | 1.9 | 13.3 | 10 | Normal |
| 8-1364 | rh8 | 1.6 | 11.3 | 10 | Normal |
| 8-1378 | rh8 | 1.3 | 10.8 | 10 | Normal |
| 8-1397 | rh8 | 1.7 | 9.2 | 10 | Normal |
| 8-1483 | 1 | 1.9 | 1.9 | 10 | Normal |
| 8-1485 | 1 | 2.0 | 1.6 | 10 | Normal |
| 9-1494 | rh8 | 1.9 | 1.4 | 10 | Normal |
| 9-1502 | rh8 | 1.8 | 0.9 | 10 | Normal |

TABLE 4
Clinical rating scale for untreated GM1 cats.

| Health Status | Score |
|---|---|
| Normal movement | 10 |
| Slight head tremor | 9 |
| Overt body tremor | 8 |
| Wide stance | 7 |
| Instability with occasional falling | 6 |
| Can stand but not ambulate | 5 |
| Cannot support weight on 4 limbs | 4 |
| Inability to enter litter box | 3 |
| Cannot support weight on front limbs | 2 |
| 20% body weight loss | 1 |

### Example 3

### AAV-Mediated Gene Therapy in GM2 Mice

(i) Separation of β-Hexosaminidase Isoforms from Mice Brain.

Isoforms of β-hexosaminidase were separated from isolated brain tissue by ion exchange chromatography (FIGS. 12A, B). Subunits of β-hexosaminidase isoforms were separated by SDS-PAGE and visualized by western blotting (FIG. 12C). Mouse cerebrum from wild type (WTBrain), untreated Sandhoff mutant (SHBrain), Tay-Sachs mutant (TSBrain), and AAV2/1 α+β co-transduced Sandhoff brain (SHBrain (2/1a+b; 1:1)) was homogenized and equal amounts of protein loaded into a Resource Q column. Fractions 1-23 were collected and analyzed for hexosaminidase activity using the substrates 4-MUG (FIG. 12A), which detects all three isozymes, and 4-MUGS (FIG. 12B) specific for Hex A and S. The fractionation of the isozymes demonstrates the absence of HexA and HexB in the untreated SD mouse brain; the presence of near normal amounts of HexB and absence of HexA in the TSD mouse and the abundance of all three isoforms in the WT. In the co-transduced SD brain all three hexosamidases are highly expressed. Assignment of each of the hexosaminidases to the peaks in panel A was corroborated by western blotting (FIG. 12C) using an antibody against human Hex A. The unfractionated cerebrum lysate from AAV2/1 α+β co-transduced SD mouse establishes the presence of mature alpha (αm) and beta (βm″a″ and βm″c″) subunits. After fractionation by ion exchange chromatography fraction number 2 contains mainly the beta subunit (Hex B), fraction number 13 shows roughly equal amounts of the alpha and the beta subunits (Hex A), and fraction number 17 only the alpha subunit (Hex S).

The profiles generates are consistent with high levels of HexA, HexB, and HexS in AAV-transduced GM2 mouse brain. Western blot analysis of fractions corresponding to peaks in the 4-MUG enzymatic profile (FIG. 12A) revealed their α-/β-subunit composition confirming their identity as HexB (Fraction 2-β/β), HexA (Fraction 13-α/β), and HexS (Fraction 17-α/α) (FIG. 12C).
(ii) β-Hexosaminidase Distribution and Microglial Immunoreactivity in SD Mice Following Bilateral Striatal and Cerebellar Infusion of AAV-HexA Constructs.

β-hexosaminidase distribution and microglial immunoreactivity were measured in two year-old AAV-treated SD mice (FIG. 13a-f, j, k); untreated SD mice at four months of age (FIGS. 13g, l, m); and heterozygous littermates (FIG. 13h, I, n, o). β-Hexosaminidase expression in the CNS was assessed by histochemical staining (FIG. 13a-i). Microglial immunoreactivity in the brain (FIGS. 13j, l, n) and spinal cord (FIGS. 13k, m, o) was assessed with CD68 antibody.

HexA distribution showed widespread distribution throughout the neuraxis of 2 year-old AAV-treated SD mice (FIG. 13a-f) following injection of hexosaminidase expression constructs bilaterally into the striatum and cerebellum. Moreover microglia activation in the CNS of these mice (FIGS. 13j, k) was dramatically reduced compared to untreated SD mice at the humane endpoint (120±6 days) (FIG. 13j-m).
(iii) β-Hexosaminidase Distribution GM2-Ganglioside Content in SD Mice Following Bilateral Intrathalamic Infusion of AAV-HexA Constructs.

One month following bilateral intrathalamic injections of AAV-Hex expression constructs in 6-8 week-old SD mice, the brains were analyzed for HexA distribution by histochemical staining (FIG. 14A), and GM2-ganglioside content (FIG. 14B). Shown is the average+1 SD (n=3). *p<0.01.

β-hexosaminidase showed widespread distribution throughout the cerebrum (FIG. 14A), and treated SD mice showed a 90% reduction in GM2-ganglioside content in the brain compared to untreated SD mice (FIG. 14B).
(iv) Quantification and Visualization of Glycosphingolipids in Mouse Brain Following Unilateral Intrastriatal Infusion of AAV-HexA Constructs.

Glycosphinglolipids (GSLs) in untransduced and transduced SD mouse brain were analyzed by high-performance thin-layer chromatography (FIGS. 15A, B) and electron microscopy (FIG. 15C). GSLs were extracted from a wild type aged 21 weeks (FIG. 15A, lanes 1, 2, and 13), an untransduced SD mouse aged 16 weeks (FIG. 15A, lanes 3, 4 and 14), or the brains of SD mice transduced with rAAVα+β at a single site in the right striatum (FIG. 15A, lanes 5-12 and 14-18). Vector was injected at 4 weeks of age; the animals were killed at 16 (FIG. 15A, lanes 5, 6, and 15), 20 (FIG. 15A, lanes 7, 8, and 16), 24 (FIG. 15A, lanes 9, 10, and 17), and 30 weeks of age (FIG. 15A, lanes 11, 12, and 18). Right (FIG. 15A, lanes 1, 3, 5, 7, 9, and 11) and left cerebrum (FIG. 15A, lanes 2, 4, 6, 8, 10, and 12) and cerebella (FIG. 15A, lanes 13-18) were dissected and individually analyzed. Pure GM1, GM2, and GA2 gangliosides and the myelin component, galactocerebroside (Galc), were used as standards (STD).

GA2 and GM2 content was quantified densitometrically and is represented as the percentage of the content in the untreated Sandhoff mouse, after correcting for loading differences, by using the internal Galc standard. Storage was diminished in all treated Sandhoff brains but increased progressively with age (FIG. 15B).

Neuronal ultrastructure in brain were evaluated by electron microscopy (FIG. 15C). Sections from wild-type (d), untransduced (c), and singly rAAVα+β-transduced SD mice (a and b). A single striatal injection of viral vector was given at 4 weeks, and the tissue harvested at 16 weeks of age. Neurons in the transduced ipsilateral cerebral cortex had no membranous cytoplasmic cell bodies (b), whereas those in the contralateral cortex (a) were distended by the storage vesicles (arrowheads in a) with distortion of the nuclei, as in untreated Sandhoff animals (c). N, nucleus. Scale bar=2 μm.

In all parts of the brain, including olfactory bulbs, brainstem, and spinal cord in SD mice receiving a single striatal injection of vectors harboring α and β, or only β subunits, at the age of 4 weeks, GM2 and GA2 ganglioside storage was reduced compared with untreated 16-week-old animals. Clearance was most evident in the ipsilateral cerebral hemisphere, but GM2 and GA2 storage continued after single injections of vector; similar effects were observed with each type of injection (FIGS. 15A, B).

Electron-dense membranous vesicles persisted in cerebral neurons contralateral to the injection site; these vesicles were absent in corresponding neurons from the ipsilateral cortex in an animal killed at 16 weeks of age and after a single striatal inoculation of rAAV2/2α+β (FIG. 15C). The relationship between transgene expression, glycosphingolipid storage and inflammation was examined in consecutive sections of brain and spinal cord from SD mice transduced with rAAV2/2α+β or rAAV2/2β alone. The number of storage cells staining with periodic acid/Schiff (PAS) reagent and the Griffonia simplicifolia isolectin B4 (GSIB4) and CD68 microglia/macrophage markers varied inversely with the activity of β-hexosaminidase. Modest expression of hexosaminidase was sufficient to reduce the number of cells expressing microglia/macrophage antigens, even though florid ganglioside storage, shown by PAS, persisted in many cells. Similar results were obtained with each vector.
(v) Relationship Between β-Hexosaminidase Activity, Glycosphingolipid Storage, and Inflammatory Cells in the Cerebral Cortex.

The relationship between β-hexosaminidase activity, glycosphingolipid storage, and inflammatory cells in the cerebral cortex of SD mice was evaluated in consecutive sections of brain and spinal cord from SD mice transduced with rAAV2/2α+β or rAAV2/2β alone (FIG. 16). Coronal sections from wild type aged 16 weeks (FIG. 13A-D), rAAV2/2α+β-transduced aged 29 weeks (humane end point) (FIGS. 16E-H), and untransduced Sandhoff mice aged 17 weeks (FIG. 16I-L) were prepared consecutively. Constructs were injected at 4 weeks of age. The β-hexosaminidase reaction product stains red (a and e) and is absent in untransduced SD mice (FIG. 16I). Glycospingolipid storage, detected by neuronal PAS staining, occurs particularly in layers IV and V of the cerebral cortex of untreated SD mice (arrowheads in FIG. 16L) but was undetectable in cortex from wild-type (FIG. 16D) or transduced SD mice (FIG. 16H). Activated microglia/macrophages were recognized by immunostaining of the cell-specific marker, CD68 (FIGS. 16B, F, and J), and by binding to isolectin B4 (FIGS. 16C, G, and K). No cells of microglia/macrophage lineage were detected in wild-type cortex (FIGS. 16B and C), and only a few were seen in transduced SD mice (arrowheads in FIGS. 16F and G). Cerebral cortex from untransduced Sandhoff mice contained numerous activated microglia and macrophages (arrowheads in FIGS. 16J and K). The number of neurons staining with PAS and the presence of cells recognized by G. simplicifolia isolectin B4 (GSIB4) and CD68 antibodies inversely depended on enzymatic activity.

The number of storage cells staining with periodic acid/Schiff (PAS) reagent and the Griffonia simplicifolia isolectin B4 (GSIB4) and CD68 microglia/macrophage markers varied inversely with the activity of β-hexosaminidase (FIG. 16). Modest expression of hexosaminidase was sufficient to reduce the number of cells expressing microglia/macrophage antigens, even though florid ganglioside storage, shown by PAS, persisted in many cells. Similar results were obtained with each vector.
(vi) Weight and Neurological Function in SD Mice Following β-Hexosaminidase Replacement Therapy.

Maintenance of body weight and rescue of neurological function was assessed in wild-type, untransduced, and transduced SD mice (FIG. 17). Transduced animals were injected at 4 weeks of age. Range of body weights in wild-type mice [blue-gray and pink stippled area for males (n=6) and females (n=7), respectively], untransduced (cyan triangles; n=1) and rAAVα-transduced (dark blue squares; n=1) SD males, untransduced (pink squares; n=5) SD females, and rAAV2/2β or rAAV2/1β-transduced SD males at four sites (open dark blue triangles; n=3) (FIG. 17A).

After therapy, SD mice gained and maintained their weight normally (FIG. 17A). Over a period of 120 days, movement frequency declined in SD mice (P=0.0108) but, in treated Sandhoff animals, remained indistinguishable from WT (P=0.1107); limb movements improved significantly after gene therapy (P<0.0001) (FIG. 17B).
(vii) Survival of SD Mice Following β-Hexosaminidase Replacement Therapy.

Animals treated by gene therapy were given either a single injection of AAV coding for human β-hexosaminidase in the right striatum or four injections (bilaterally in the striatum and cerebellum) at four weeks of age. Untreated SD mice reached their pre-defined humane endpoint at around 120 days of age, those treated at a single site at 200 days, but about 25% of the animals given four injections, at this particular vector dose, were still alive at one year of age (FIG. 18).
(viii) AAV-Treated SD Mice Display Improved Performance in the Inverted Screen Test Compared to Untreated SD Mice.

The performance of AAV-treated SD mice in the inverted screen test was compared to that of untreated SD (MT) and heterozygote (WT) control mice. For AAV2/1-treated mice, Hex subunits were tested with (α1, β1) or without (α4, β4) a carboxyl-terminal fusion of the HIV Tat protein transduction domain (FIG. 19A). AAV constructs were infused bilaterally into the thalamus and deep cerebellar nuclei at three months of age. Performance of AAV2/rh8-treated mice compared to untreated SD (MT) and heterozygote (WT) control mice (FIG. 19B). Two comparisons were performed between AAV2/rh8-treated SD mice (123 and 246 days of age) and untreated SD mice (123 days of age) using the Mann-Whitney test. A significant difference in performance in the Inverted Screen Test at P<0.05 was found when comparing the two groups at 123 days of age.
(ix) AAV-Treated SD Mice Display Sustained Performance in Accelerating Rotarod and Barnes Maze Tests Over Time.

The performance of AAV2/1-treated SD mice in the accelerating rotarod test was compared to that of untreated SD (MT) and heterozygote (WT) control mice. For AAV2/1-treated mice, Hex subunits were tested with (α1, β1) or without (α4, β4) a carboxyl-terminal fusion of the HIV Tat protein transduction domain (FIG. 20A). AAV constructs were infused bilaterally into the thalamus and deep cerebellar nuclei at one month of age. Performance of AAV2/rh8-treated mice compared to untreated SD (MT) and heterozygote (WT) control mice (FIG. 20B). Two comparisons were performed between AAV2/rh8-treated SD mice (123 and 246 days of age) and untreated SD mice (123 days of age) using the Mann-Whitney test. No significant differences were found in performance in the Accelerating Rotarod test at P<0.05 between the two groups (FIG. 20).

The performance of AAV2/1-treated SD mice in the Barnes maze test was compared to that of untreated SD (MT) and heterozygote (WT) control mice. For AAV2/1-treated mice, Hex subunits were tested with (α1, β1) or without (α4, β4) a carboxyl-terminal fusion of the HIV Tat protein transduction domain (FIG. 21A). AAV constructs were infused bilaterally into the thalamus and deep cerebellar nuclei at one month of age. Performance of AAV2/rh8-treated mice compared to untreated SD (MT) and heterozygote (WT) control mice (FIG. 21B). Two comparisons were performed between AAV2/rh8-treated SD mice (123 and 246 days of age) and untreated SD mice (123 days of age) using the Mann-Whitney test. The performance of AAV2/rh8-treated SD mice was significantly (P<0.05) better than untreated SD mice at either age analyzed (FIG. 21).
(x) Quantification of β-Hexosaminidase Enzymatic Activity in Cerebrum and Cerebellum of Heterozygous (Hexb+/−), SD (Hexb−/−), and AAV-Treated SD Mice.

For treated animals, AAV constructs were infused bilaterally into the thalamus and deep cerebellar nuclei at one month of age. Results are shown in FIG. 22. Activities were measured in frozen tissue sections using 4-methyumbelliferyl-N-acetyl-β-D-glucosaminide as the substrate. Values are expressed as the mean±SEM. N=3, 4, and 6 mice per group for heterozygote (HZ), untreated SD (KO), and AAV-treated SD (KO) mice, respectively. Asterisks denote statistical significance with a p-value <0.05 using a student's two-tailed t-test.

Total β-Hexosaminidase specific activity in AAV-treated SD mouse cerebrum was significantly greater than untreated SD cortex in sections R2 and R3 (FIG. 22a). In all sections assayed, Hex activity was equal to or greater than the activity in the HZ mice. The most pronounced difference in enzymatic activity was seen in sections R3 and R4. Hex activity was significantly greater in cerebellum of AAV-treated SD mouse than in untreated SD mice (FIG. 22b), and was 85-fold greater than in cerebellum of HZ mice.
(xi) AAV-Mediated β-Hexosaminidase Expression Reduces Total Ganglioside Content and Corrects GM2 Storage in SD Mouse Cerebrum and Cerebellum.

AAV constructs were infused bilaterally into the thalamus and deep cerebellar nuclei at one month of age. Brains were harvested at 8 months of age (or humane endpoint for untreated SD mice) and ganglioside cerebral (FIG. 23) and cerebellar (FIG. 24) content was measured by HPTLC. Approximately 1.5 μg of sialic acid was spotted per lane from pooled right brain sections (FIGS. 23A, 24 A). Total sialic acid content quantified using the resorcinol assay (FIGS. 23B, 24B). GM2 content quantified via densitometric scanning of the HPTLC plate in the A panels (FIGS. 23C, 24C). Values are expressed as mean±SEM. N=3, 4, and 6 mice per group for HZ, untreated SD (KO), and AAV-treated SD (AAV) mice, respectively. Asterisks denote a statistically significant difference (p<0.001) from the untreated SD (KO) mice using one-way ANOVA.

Total ganglioside and GM2 content was significantly lower in the cerebrum of AAV2/rh8-treated SD mice compared to untreated SD controls (FIG. 23). Cerebral ganglioside content in the treated mice was corrected to levels indistinguishable from HZ controls (FIG. 23). AAV treatment reduced total cerebellar ganglioside content in SD mice to the same levels as HZ controls (FIG. 24). Although GM2 content was significantly reduced compared to untreated SD mice (FIGS. 24A, C), there were two samples where substantial GM2 storage remained (FIG. 24A). Notably, the sample (#80336) with the least cerebellar enzymatic activity relative to the other AAV-treated samples had a ganglioside profile similar to that of the untreated SD mice. Also, though corrected for GM2 content in cerebrum, its cerebrum Hex activity was the lowest of all AAV-treated samples in 3 of 4 sections assayed.
(xii) Influences of AAV Gene Therapy on Myelin-Associated Cerebrosides and Sulfatides in SD Mouse Brain.

AAV constructs were infused bilaterally into the thalamus and deep cerebellar nuclei at one month of age. Brains were harvested at 8 months of age (or humane endpoint for untreated SD mice) and the cerebroside and sulfatide content measured by HPTLC (FIG. 25).

Neutral and Acidic lipids purified from right cortex (FIG. 25A) and right cerebellum (FIG. 25B) were spotted on HPTLC at 70 ug and 200 ug/mg dry tissue weight, respectively. Values for cerebrosides and sulfatides were taken from densitometric scanning of HPTLC plates (data not shown). Values are expressed as mean±SEM. N=3, 4, and 6 mice per group for HZ, untreated SD (KO), and AAV-treated SD (AAV) mice, respectively. Asterisks denote a statistically significant difference (p<0.05 and p<0.01, respectively) from the untreated SD (KO) mice using one-way ANOVA.

AAV-treatment significantly increased myelin-associated lipids (cerebrosides and sulfatides) in cerebrum (FIG. 25A) and cerebellum (FIG. 25B) compared to untreated SD mice. Sulfatide content was almost completely restored to the HZ levels in cerebellum. It is known that ganglioside storage reduces cerebrosides and sulfatides. Our results suggest that the AAV-treatment largely corrected both primary GM2 storage and secondary damage to myelin in the SD mice.
(xiii) Effect of AAV-Treatment on Disease Marker Gene Expression in the CNS of SD Mice.

Quantitative PCR was used to analyze expression levels of CD68, IL-1β, Lgal3(Mac2), Mip1-α and TNF-α which have been shown to be elevated in the CNS of SD mice. AAV constructs were infused bilaterally into the thalamus and deep cerebellar nuclei at three months of age. Cerebrum, cerebellum, brain stem, and anterior and posterior spinal cord segments were analyzed for 8 month-old AAV2/rh8-treated SD mice, untreated SD mice at humane endpoint, and 8-month old untreated heterozygote controls (FIG. 26). Expression levels of disease marker genes in AAV2/rh8-treated SD mice at 8 months of age (red bars), and untreated SD mice at humane endpoint (black bars) normalized for levels in 8 month-old untreated heterozygote animals. Show is the mean±SEM. N=3 for each structure analyzed. Dotted line indicates normal expression levels. Asterisks denote statistical significance with a p-value <0.05 using a student's one-tailed t-test.

There was no significant reduction in expression levels of most marker genes in the cerebrum and cerebellum of AAV2/rh8-treated SD mice compared to untreated SD mice, with the exception of Mip1-α (FIGS. 26A, B). By contrast, AAV-treatment significantly reduced expression levels of most genes (P<0.05), except IL-1β, in the brain stem, anterior and posterior segments of the spinal cord (FIG. 26C-E).

### Example 4

### AAV-Mediated Gene Therapy in GM2 Cats

(ii) Widespread Distribution of β-Hexosaminidase in GM2 Cat Brain Following Intracranial Infusion of AAV-HexA Constructs

Pre-symptomatic 4-6 week-old GM2 cats received bilateral infusions of AAV2/rh8 vector formulation (ratio=1:1; titer: 1.16E13 gc/ml for each vector) in the thalamus (70 μl/side) and deep cerebellar nuclei (24 μl/side) for a total volume of 188 μl and a total combined AAV vector dose of 4.4E12 gc (high dose cohort). A second cohort of six GM2 cats received a 10-fold lower dose delivered in the same total volume (Cats 7-773, 11-777, 11-778, 7-787, 7-789, 7-793, underlined in shaded boxes in Table 5). Three AAV-treated GM2 cats in the highest-dose cohort (4.4E12 gc) were euthanized at 16 weeks post-injection for biochemical analysis of enzyme distribution and quantification of GM2-ganglioside content throughout the CNS (Cats 11-762, 7-770, 7-774).

Histological and biochemical analysis of hexosamindase distribution in the brain of these animals showed enzyme present throughout the cerebrum and cerebellum at levels higher than normal in all regions analyzed (range: 2.0-50.7 fold above normal) (FIG. 27).
(iii) Decreased GM2-Ganglioside Storage in the Brain of AAV-Treated GM2 Cats

In untreated GM2 cats, neurochemical analysis of gangliosides and other lipids in the brain showed that total ganglioside sialic acid concentration (FIG. 28B) and the levels of GM2-ganglioside (FIG. 28C) were significantly elevated in the cortex, cerebellum, striatum, and thalamus compared to normal cats (FIG. 28). AAV gene therapy reduced sialic acid and GM2-ganglioside content in all regions of the GM2 cat brain examined (gray bars in FIGS. 28B, 28C, and FIG. 29).
(iv) Restoration of Myelin-Associated Lipids in the Brain of AAV-Treated GM2 Cats

In untreated GM2 cat brain, the levels of myelin-enriched cerebrosides were significantly lower than in normal cat brain as previously described (FIG. 28D). These reductions signify myelin abnormalities secondary to GM2-ganglioside storage. Our findings in AAV-treated GM2 cats show that cerebrosides were restored to near normal levels in most regions of the brain (FIG. 28D, gray bars).
(v) Distribution of β-Hexosaminidase in the Spinal Cord of AAV-Treated GM2 Cats

The spinal cord gray matter of AAV-treated GM2 cats stained strongly for Hex activity (FIG. 30A), and biochemical quantification confirmed that total hexosaminidase activity in cervical (range: 1.6-17 fold) and lumbar (range: 4.3-12.6 fold) spinal cord was considerably elevated over normal values (FIG. 30B).
(vi) Decreased GM2-Ganglioside Storage in the Spinal Cord of AAV-Treated GM2 Cats

AAV treatment reduced total ganglioside sialic acid concentration (FIG. 30C), GM2-ganglioside (FIG. 30D), and GA2 (FIG. 30E) in both regions of the spinal cord.
(vii) Enhanced Survival of AAV-Treated GM2 Cats

All GM2 cats in the highest dose cohort (4.4E12 gc), except the first injected cat (7-714), showed excellent health and ambulatory but with hindlimb weakness/paresis (Table 5). Cat 7-714 died suddenly at ˜16 months of age after a long period of pronounced hindlimb weakness/paresis. All AAV-treated GM2 cats in the lower dose cohort (4.4E11 gc) developed disease symptoms such as whole body tremor (CRS 8), but the onset and progression were delayed (Table 5). In untreated Sandhoff disease cats (GM2 cats) the disease progressed rapidly with average survival to 4.5±0.5 months of age (n=11).

Treatment of GM2 cats with AAV-mediated βHex expression resulted in suppression of clinical symptoms of GM2 gangliosidosis. The human endpoint for untreated GM2 cats is defined by the subject's inability to support weight on its forelimbs over two consecutive days or the loss of 20% of maximal body weight (scores 2 and 1, respectively, on the clinical rating scale given in Table 6). GM2 cats treated with AAV-βHex constructs scored as high 8-10 in the clinical rating scale out to >12 months post-treatment. Clinical ratings were assigned according to the scale given Table 6.

TABLE 5
AAV-treated GM2 cats

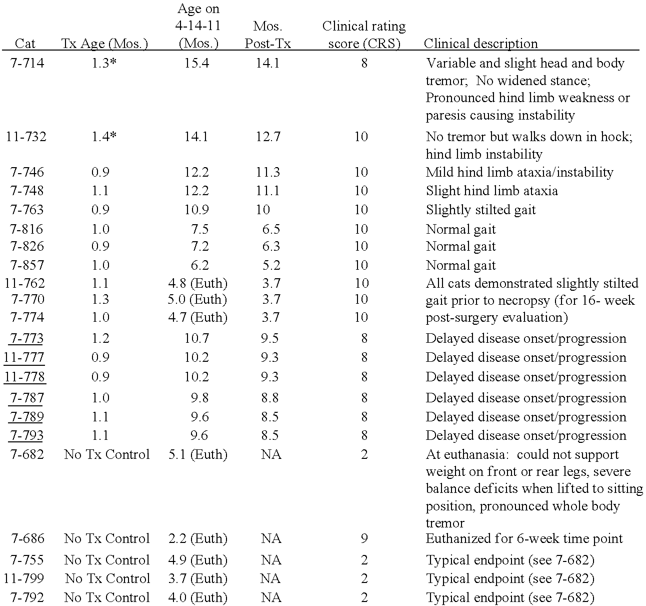

*Teatment (Tx) was delayed past one month of age due to ppor health and poor surgical risk. Tx consisted of bilateral injection of thalamus (8.2 × 10¹¹ gc. per vector per side) and deep cerebellar nuclei (2.3-2.8 × 10¹¹ gc per vector per side) with a 1:1 ratio of the following vectors: AAV2/rh8-CBA-fHEXA-WPRE and AAV2/rh8-CBA-fHEXB-WPRE. Combined vector dose for each cat was 4.2-4.4 × 10¹² g.e., except for cats treated with one-tenth the dose (underlined numbers in shaded boxes).
“Euth” refers to the age at euthanasia.
NA = not applicable.
NOTE: The humane endpoint for untreated GM2 cats is 4.5 ± 0.46 months (n = 11).

TABLE 6
Clinical rating scale for GM2 cats.

| Health Status | Score | Age (Mos.) |
|---|---|---|
| Normal movement | 10 | <1.6 |
| Slight head tremor | 9 | 1.7 |
| Overt body tremor | 8 | 2.5 |
| Wide stance | 7 | 2.7 |
| Instability with occasional falling | 6 | 2.9 |
| Can stand but not ambulate | 5 | 3.5 |
| Cannot support weight on four limbs | 4 | 3.9 |
| Inability to enter litter box | 3 | 4.3 |
| Cannot support weight on front legs | 2 | 4.5 |
| 20% body wt. loss - Humane endpoint | 1 | >4.5 |

AAV-treated cats were weighed at least every 2 weeks, and plots of weight versus age were constructed (FIG. 31). To the data points were added a best fit linear trend line (Microsoft Excel), and the slope of the trend line was calculated to determine the growth rate. Because growth rates decrease with age, rates were calculated for 2 separate age ranges: 5-18 weeks and 5-24 weeks. The left-hand panel depicts growth rates in kg/week, while the right-hand panel provides an example of typical growth curves (Normal, 7-735; GM2+AAV, 11-732; GM2, 7-682). AAV-treated

The body weight of AAV-treated GM2 cats remained stable or increased after 3.75 months of age (FIG. 31). From birth to humane endpoint, untreated GM2 cats weighed less and grew more slowly than their normal or heterozygote littermates. Also, untreated GM2 cats typically lost weight just before reaching the neurological humane endpoint. For these reasons, weight and growth rate are considered reliable indicators of disease progression. The growth rate of AAV-treated GM2 cats is intermediate to normal and untreated GM2 cats, suggesting partial normalization of the disease process responsible for reduced weight and growth rate in feline GM2 gangliosidosis (FIG. 31).

Magnetic resonance images were taken on all treated and control cats prior to surgery and at 6 and 16 weeks post-injection. Images were analyzed by an independent veterinary radiologist blinded to experimental treatment. MR results were consistent with other clinical analyses, revealing in AAV-treated GM2 cats brain deterioration intermediate to untreated GM2 and normal cats (FIG. 32). For example, an untreated GM2 cat (7-682) at 5.1 months of age demonstrated progressive deepening of and markedly prominent sulci compared with previous MR images. The lateral ventricles were of increased size (3 mm) compared with previous images, and there was persistence of increased signal intensity of the white matter and internal capsule, particularly evident on proton density and FLAIR images. T1 weighted images for 7-682 demonstrated bilateral decreased signal at the level of the geniculate bodies, which appeared to be well-demarcated compared to normal and previous images. By comparison, the oldest AAV-treated GM2 cat (7-714) at 4.9 months of age demonstrated sulci that were marginally deepened compared to a normal, age-matched cat, but were not as deep as the untreated GM2 cat. Likewise, the lateral ventricles of the AAV-treated cat were slightly dilated compared to normal (1.5 mm) but remarkably normalized compared to the untreated GM2 cat. White matter signal hypointensity relative to gray matter was largely but not completely preserved in the treated GM2 cat. Interestingly, the geniculate bodies in the treated GM2 cat were well-demarcated and of decreased signal intensity, and similar in appearance to the untreated GM2 cat.

Initial gait analyses of treated GM2 cats were performed on a GAITRite High Resolution Platinum (CatMat) 6′ Walkway System (CIR Systems, Havertown, Pa.). Normal (7-711, 5.9 mos) and AAV-treated GM2 (7-714, 5.6 mos) cats were evaluated. As shown in FIG. 33, AAV-treated GM2 cat 7-714 exhibited mild but quantifiable gait abnormalities at 5.6 months of age, >1 month past the humane endpoint for untreated GM2 cats. Of interest is the shorter than normal stride length for 7-714 and shorter than normal reach, especially on the left side (note overlap of blue and green sensor images). [Reach is defined as the distance from heel center of the hind paw to heel center of the previous fore paw.] Other left-sided abnormalities were detected by the gait analysis mat (panel B) that were not readily apparent upon neurological examination. Unfortunately, this one-of-a-kind gait analysis system was being manufactured when untreated GM2 cats were still capable of ambulation, so data has not yet been collected from untreated GM2 cats.

### Example 5

### Determination of Effective Vector Dose

This example will illustrate determination of the minimum dose of AAV vector formulation that results in >75% decrease in GM2-ganglioside content in the brain or cerebellum after bilateral thalamic or cerebellar (deep cerebellar nuclei, dcn) injections, respectively. This will be considered the AAV vector most effective dose (MED). This study will be composed of 2 arms with 7 groups each (Table 7). One-month old SD mice will receive bilateral injections of AAV vectors, or vehicle, in the thalamus (1 μl per site) or deep cerebellar nuclei (0.5 μl per site). In each arm of the experiment (thalamic or cerebellar injections), Groups 1-4 will be injected with increasing AAV vector doses of 0.1, 0.3, 1, and 3×10¹⁰ vg in the thalamus, or 0.05, 0.15, 0.5, and 1.5×10¹⁰ vg in the cerebellum. Indicated doses refer to the dose of each AAV vector in the formulation. Group 5 will serve as a positive control and will be injected with 1 μl of 1:1 AAV vector formulation of the previously validated AAV-αTat AAV-βTat (1×10¹⁰ vg per vector). Control groups 6 and 7 will be injected with AAV empty vector (without transgene at a dose of 3×10¹⁰ vg), or with vehicle (PBS), respectively. A group of 4 untreated SD mice will be common to both Arms, and age-matched heterozygote (HZ) mice will be used as controls for biochemical and histological assays (n=4). Total number of mice in this study will be: n=4 per group×2 arms×7 groups/arm=56 experimental SD mice+4 untreated SD mice+4 HZ mice. Each group will be composed of 2 males and 2 female SD mice. Body weights will be measured twice weekly. Mice will be sacrificed if body weight declines by >15%, and analyzed for evidence of neuropathology.

The experimental endpoint for these SD mice will be at 2 months post-injection (3 months of age). One brain hemisphere, hemi-cerebellum, and spinal cord will be used for biochemical quantification of enzymatic activity and GM2-ganglioside levels. Brain hemispheres will be divided into 5 coronal slabs (˜2 mm thick) and measure enzymatic activity in each slab with 4-MUG and 4-MUGS. The hemi-cerebellum will be analyzed as a single sample. Following enzymatic activity measurements, all brain hemisphere slab lysates from each animal will be combined for measurement of GM2-ganglioside levels by high-performance thin layer chromatography (HPTLC). The spinal cord will be cut into 2-3 mm transverse sections, and every other section will be used for enzymatic activity assays. The other set of spinal cord sections will be used for histological analyses. The following histological analyses will be performed in the opposite hemisphere, hemi-cerebellum, and spinal cord in groups showing ≧75% reduction in GM2-ganglioside content in the brain or cerebellum, depending on the experimental arm, compared to GM2 animals in control Groups 5-7: 1) β-hexosaminidase distribution by histochemical staining; 2) AAV vector distribution by in situ hybridization; 3) Hematoxylin and eosin staining for overall neuropathological evaluation; 4) Microglial activation by immunohistochemical staining with anti-MHC II antibody or staining with Griffonia simplicifolia isolectin B4 (GSIB4) 27, 28, 124; 5) Presence of inflammatory infiltrates at the injection site by immunohistochemistry with antibodies to CD68, CD4, and CD8. In addition the following organs will be analyzed for the presence of AAV vector genomes by real-time PCR on genomic DNA: heart, liver, muscle (right quadriceps), spleen, lung, diaphragm, right eye, right kidney, prostate, testis, ovaries, thymus and sciatic nerve.

It is anticipated that at least one dose of AAV vector formulation will lead to ≧75% reduction in GM2-ganglioside content (≦82 μg sialic acid/100 mg dry weight) in the cerebrum and cerebellum compared with control GM2 animals. (Control groups 6 and 7 in either arm of the experiment are expected to have a GM2-ganglioside content of 327±27 μg sialic acid/100 mg dry weight, i.e., the level of GM2 in untreated mice.) As discussed previously (see above), thalamic infusion of AAV vectors in adult GM1-gangliosidosis mice resulted in large decreases in GM1-ganglioside content in cerebrum, cerebellum and spinal cord. Thus, it is expected that in SD mice receiving thalamic injections there will be a statistically significant decrease in GM2-ganglioside content in the cerebellum and spinal cord. However, cerebellar injections are expected to have a larger effect on GM2-ganglioside content in cerebellum than thalamic injections. In the event that one or more doses meet our criterion of >75% reduction in GM2-ganglioside content, the dose will be selected that shows maximal effect in the absence of neurotoxicity at the site of injection (neuronal loss, evidence of neuronophagia, vascular cuffing, and presence of inflammatory infiltrates).

Based on prior injections of AAV2/1 and AAV2/2 vectors in the brain of SD mice, AAV-associated neurotoxicity at the site of injection is not anticipated. Rather, a statistically significant decrease in microglial activation in the spinal cord and brain stem in SD mice receiving thalamic injections of AAV vector formulation and showing >75% reduction in GM2-ganglioside content in the cerebrum compared to control SD mice (Groups 6-7) is expected. Untreated SD mice start to show clear histological evidence of microglial activation by 2 months of age. Finally, it is not anticipated that AAV vector genomes will be present in most peripheral organs, except the eye. Because the injections are thalamic, it is possible that some AAV vector may be transported to the retinal ganglion cell bodies via axonal retrograde transport from the lateral geniculate nucleus in the thalamus. There is evidence suggesting that AAV2/1 vectors may be transported to distant sites via axonal transport.

An exemplary experimental scheme for determining effective AAV vector dose is shown in Table. 7.

TABLE 7
Determination of effective vector dose.

| | | Thalamus | Cerebellum |
|---|---|---|---|
| | | Arm 1 (n = 4) | Arm 2 (n = 4) |
| Group | Vector formulation | Dose (×10¹⁰/kg) | Dose (×10¹⁰/kg) |
| 1 | | 0.1 | 0.05 |
| 2 | AAVα + AAVβ | 0.3 | 0.15 |
| 3 | | 1.0 | 0.5 |
| 4 | | 3.0 | 1.5 |
| 5 | AAV-αTat + AAV-βTat | 1.0 | 0.5 |
| 6 | AAV-empty | 3.0 | 1.5 |
| 7 | PBS only | 0.0 | 0.0 |

### Example 6

### AAVrh.8-Mediated Gene Therapy in Humans

This example will illustrate use of the methods described herein in providing AAVrh.8-mediated gene replacement therapy in human subjects in need thereof. Depending on the method subject will be suffering from a lysosomal storage disorder comprising Tay-Sachs Disease, Sandhoff Disease, or GM1-gangliosidosis. Depending on the embodiment, the subject will be diagnosed as having, suspected of having, or predisposed to having one of said lysosomal storage disorders. Depending on the embodiment, the subject will be administered a pharmaceutical composition comprising AAVrh.8 expression constructs encoding β-hexosaminidase α and β subunits, or encoding acid β-galactosidase. Depending on the embodiment, the subject will be administered the AAVrh.8 composition for the purpose of treating, delaying the onset of, or reducing the severity of Tay-Sachs Disease, Sandhoff Disease, or GM1-gangliosidosis. Depending on the embodiment, the subject will be administered the AAVrh.8 composition for the purpose of achieving widespread distribution of β-hexosaminidase α and β subunits or acid β-galactosidase in the brain.

The AAVrh.8 composition will be administered to the subject intracranially under sterile conditions as appropriate for the procedure. The effective dose of the AAVrh.8 composition will be empirically determined using cell or animal models. Depending on the embodiment, encoding β-hexosaminidase or acid β-galactosidase activity will be evaluated in the subject following administration of the AAvrh.8 composition. Depending on the embodiment, evaluation of enzyme activity will comprise evaluation of symptoms associated with Tay-Sachs Disease, Sandhoff Disease, or GM1-gangliosidosis, or biochemical assessment β-hexosaminidase or acid β-galactosidase activity. Based on said evaluation, the subject may receive additional administrations of the AAVrh.8 composition to achieve or maintain the desired effect.

It is anticipated that the methods described herein will be effective methods for the treatment of lysosomal disorders. Specifically, it is anticipated that intracranial delivery of an effective dose of AAVrh.8 β-hexosaminidase α and β subunits or acid β-galactosidase in a human subject diagnosed as having, suspected of having, or predisposed to having Tay-Sachs Disease, Sandhoff Disease, or GM1-gangliosidosis will significantly diminish manifestations of these disorders. The disclosed methods will reduce symptoms associated with the disorders such as neurological deficits, and will reduce the level of GM1 or GM2 ganglioside storage in the brain. In the context of prophylactic administrations, the disclosed methods will delay the onset of, reduce the likelihood of, reduce the severity of these disorders.

### EQUIVALENTS

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and compositions within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled. It is to be understood that this invention is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible sub ranges and combinations of sub ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub ranges as discussed above. Finally, as will be understood by one skilled in the art, a range includes each individual member. Thus, for example, a group having 1-3 units refers to groups having 1, 2, or 3 units. Similarly, a group having 1-5 units refers to groups having 1, 2, 3, 4, or 5 units, and so forth.

All references cited herein are incorporated by reference in their entireties and for all purposes to the same extent as if each individual publication, patent, or patent application was specifically and individually incorporated by reference in its entirety for all purposes.

Other embodiments are set forth within the following claims.

### REFERENCES

1. Guidotti, J., Akli, S., Castelnau-Ptakhine, L., Kahn, A. & Poenaru, L. Retrovirus-mediated enzymatic correction of Tay-Sachs defect in transduced and non-transduced cells. Hum Mol Genet. 7, 831-8 (1998).
2. Teixeira, C. A., Sena-Esteves, M., Lopes, L., Sa Miranda, M. C. & Ribeiro, M. G. Retrovirus-mediated transfer and expression of beta-hexosaminidase alpha-chain cDNA in human fibroblasts from G(M2)-gangliosidosis B1 variant. Hum Gene Ther 12, 1771-83 (2001).
3. Guidotti, J. E. et al. Adenoviral gene therapy of the Tay-Sachs disease in hexosaminidase A-deficient knock-out mice. Hum Mol Genet. 8, 831-8 (1999).
4. Cachon-Gonzalez, M. B. et al. Effective gene therapy in an authentic model of Tay-Sachs-related diseases. Proc Natl Acad Sci USA 103, 10373-8 (2006).
5. Meikle, P. J., Hopwood, J. J., Clague, A. E. & Carey, W. F. Prevalence of lysosomal storage disorders. JAMA 281, 249-254 (1999).
6. Gravel, R. A. et al. The GM2 gangliosidoses. in The Metabolic and Molecular Bases of Inherited Disease, Vol. 2 (eds. Scriver, C. R., Beaudet, A. L., Sly, W. S. & Valle, D.) 2839-2879 (McGraw-Hill, Inc., New York, 1995).
7. Tay, W. Symmetrical changes in the region of the yellow spot in each eye of an infant. 1, 155-157 (1881).
8. Mahuran, D. J. Beta-hexosaminidase: biosynthesis and processing of the normal enzyme, and identification of mutations causing Jewish Tay-Sachs disease. Clin. Biochem. 28, 101-106 (1995).
9. Mahuran, D. J. Biochemical consequences of mutations causing the GM2 gangliosidoses. [Review][239 refs]. Biochimica et Biophysica Acta 1455, 105-138 (1999).
10. Sango, K. et al. Mouse models of Tay-Sachs and Sandhoff diseases differ in neurologic phenotype and ganglioside metabolism. Nature Genetics 11, 170-176 (1995).
11. Phaneuf, D. et al. Dramatically different phenotypes in mouse models of human Tay-Sachs and Sandhoff diseases. Human Molecular Genetics 5, 1-14 (1996).
12. Jeyakumar, M. et al. An inducible mouse model of late onset Tay-Sachs disease. Neurobiology of Disease 10, 201-210 (2002).
13. Miklyaeva, E. I. et al. Late onset Tay-Sachs disease in mice with targeted disruption of the Hexa gene: behavioral changes and pathology of the central nervous system. Brain Research 1001, 37-50 (2004).
14. Cork, L. C. et al. GM2 ganglioside lysosomal storage disease in cats with beta-hexosaminidase deficiency. Science 196, 1014-1017 (1977).
15. Baker, H. J., Reynolds, G. D., Walkley, S. U., Cox, N. R. & Baker, G. H. The gangliosidoses: comparative features and research applications. Veterinary Pathology 16, 635-649 (1979).
16. Baker, H. J., Smith, B. F., Martin, D. R. & Foureman, P. Molecular diagnosis of gangliosidoses: a model for elimination of inherited diseases in pure breeds. in Consultations in Feline Internal Medicine (ed. August, J. R.) 615-620 (W.B. Saunders Co., Philadelphia, 2001).
17. Martin, D. R. et al. An inversion of 25 base pairs causes feline GM2 gangliosidosis variant 0. Experimental Neurology 187, 30-37 (2004).
18. Rattazzi, M. C., Appel, A. M. & Baker, H. J. Enzyme replacement in feline GM2 gangliosidosis: catabolic effects of human beta-hexosaminidase A. Progress in Clinical & Biological Research 94, 213-220 (1982).
19. Rattazzi, M. C., Lanse, S. B., McCullough, R. A., Nester, J. A. & Jacobs, E. A. Towards enzyme replacement in GM2 gangliosidosis: organ disposition and induced central nervous system uptake of human beta-hexosaminidase in the cat. 16, 179-193 (1980).
20. Walkley, S. U., Baker, H. J. & Rattazzi, M. C. Initiation and growth of ectopic neurites and meganeurites during postnatal cortical development in ganglioside storage disease. Brain Research Developmental Brain Research. 51, 167-178 (1990).
21. Walkley, S. U., Baker, H. J., Rattazzi, M. C., Haskins, M. E. & Wu, J. Y. Neuroaxonal dystrophy in neuronal storage disorders: evidence for major GABAergic neuron involvement. Journal of the Neurological Sciences 104, 1-8 (1991).
22. Walkley, S. U., Wurzelmann, S., Rattazzi, M. C. & Baker, H. J. Distribution of ectopic neurite growth and other geometrical distortions of CNS neurons in feline GM2 gangliosidosis. 510, 63-73 (1990).
23. Wood, P. A., McBride, M. R., Baker, H. J. & Christian, S. T. Fluorescence polarization analysis, lipid composition, and Na+, K+-ATPase kinetics of synaptosomal membranes in feline GM1 and GM2 gangliosidosis. Journal of Neurochemistry 44, 947-956 (1985).
24. Zhou, J., Shao, H., Cox, N. R., Baker, H. J. & Ewald, S. J. Gangliosides enhance apoptosis of thymocytes. Cellular Immunology 183, 90-98 (1998).
25. Okada, S. & O'Brien, J. S. Tay-Sachs disease: generalized absence of a beta-D-N-acetylhexosaminidase component. Science 165, 698-700 (1969).
26. Sandhoff, K., Andreae, U. & Jatzkewitz, H. Deficient hexozaminidase activity in an exceptional case of Tay-Sachs disease with additional storage of kidney globoside in visceral organs. Life Sci 7(6), 283-8 (1968).
27. O'Brien, J. S. & Norden, A. G. Nature of the mutation in adult beta-galactosidase deficient patients. American Journal of Human Genetics 29, 184-190 (1977).
28. Hickman, S. & Neufeld, E. F. A hypothesis for I-cell disease: defective hydrolases that do not enter lysosomes. Biochemical & Biophysical Research Communications 49, 992-999 (1972).
29. Hickman, S., Shapiro, L. J. & Neufeld, E. F. A recognition marker required for uptake of a lysosomal enzyme by cultured fibroblasts. Biochemical & Biophysical Research Communications 57, 55-61 (1974).
30. Dahms, N. M., Lobel, P. & Kornfeld, S. Mannose 6-phosphate receptors and lysosomal enzyme targeting. J Biol Chem 264, 12115-12118 (1989).
31. Barranger, J. A. & O'Rourke, E. Lessons learned from the development of enzyme therapy for Gaucher disease J Inherit Metab Dis 24 Suppl 2, 89-96; discussion 87-8 (2001).
32. Burrow, T. A., Hopkin, R. J., Leslie, N. D., Tinkle, B. T. & Grabowski, G. A. Enzyme reconstitution/replacement therapy for lysosomal storage diseases. Curr Opin Pediatr 19, 628-635 (2007).
33. Jacobs, J. F., Willemsen, M. A., Groot-Loonen, J. J., Wevers, R. A. & Hoogerbrugge, P. M. Allogeneic BMT followed by substrate reduction therapy in a child with subacute Tay-Sachs disease. Bone Marrow Transplant 36, 925-6 (2005).
34. Hodges, B. L. & Cheng, S. H. Cell and gene-based therapies for the lysosomal storage diseases. Curr Gene Ther 6, 227-41 (2006).
35. Sands, M. S. & Davidson, B. L. Gene therapy for lysosomal storage diseases. Mol Ther 13, 839-49 (2006).
36. Mandel, R. J. et al. Recombinant adeno-associated viral vectors as therapeutic agents to treat neurological disorders. Mol Ther 13, 463-83 (2006).
37. Lowenstein, P. R., Mandel, R. J., Xiong, W. D., Kroeger, K. & Castro, M. G. Immune Responses to Adenovirus and Adeno-Associated Vectors Used for Gene Therapy of Brain Diseases The Role of Immunological Synapses in Understanding the Cell Biology of Neuroimmune Interactions. Curr Gene Ther 7, 347-360 (2007).
38. Vite, C. H. et al. Effective gene therapy for an inherited CNS disease in a large animal model. Ann Neurol 57, 355-64 (2005).
39. Ciron, C. et al. Gene therapy of the brain in the dog model of Hurler's syndrome Ann Neurol 60, 204-13 (2006).
40. During, M. J., Kaplitt, M. G., Stern, M. B. & Eidelberg, D. Subthalamic GAD gene transfer in Parkinson disease patients who are candidates for deep brain stimulation. Hum Gene Ther 12, 1589-91 (2001).
41. Kaplitt, M. G. et al. Safety and tolerability of gene therapy with an adeno-associated virus (AAV) borne GAD gene for Parkinson's disease: an open label, phase I trial. Lancet 369, 2097-105 (2007).
42. Janson, C. et al. Clinical protocol. Gene therapy of Canavan disease: AAV-2 vector for neurosurgical delivery of aspartoacylase gene (ASPA) to the human brain. Hum Gene Ther 13, 1391-412 (2002).
43. McPhee, S. W. et al. Immune responses to AAV in a phase I study for Canavan disease. J Gene Med 8, 577-88 (2006).
44. Crystal, R. G. et al. Clinical protocol. Administration of a replication-deficient adeno-associated virus gene transfer vector expressing the human CLN2 cDNA to the brain of children with late infantile neuronal ceroid lipofuscinosis. Hum Gene Ther 15, 1131-54 (2004).
45. Kaplitt, M. G. et al. Safety and tolerability of gene therapy with an adeno-associated virus (AAV) borne GAD gene for Parkinson's disease: an open label, phase I trial. The Lancet 369, 2097-2105 (2007).
46. Feigin, A. et al. Modulation of metabolic brain networks after subthalamic gene therapy for Parkinson's disease. Proc Natl Acad Sci USA 104, 19559-64 (2007).
47. Taylor, R. M. & Wolfe, J. H. Decreased lysosomal storage in the adult MPS VII mouse brain in the vicinity of grafts of retroviral vector-corrected fibroblasts secreting high levels of beta-glucuronidase. Nat Med 3, 771-4 (1997).
48. Cearley, C. N. & Wolfe, J. H. Transduction characteristics of adeno-associated virus vectors expressing cap serotypes 7, 8, 9, and Rh10 in the mouse brain. Mol Ther 13, 528-37 (2006).
49. Cearley, C. N. & Wolfe, J. H. A single injection of an adeno-associated virus vector into nuclei with divergent connections results in widespread vector distribution in the brain and global correction of a neurogenetic disease. J Neurosci 27, 9928-40 (2007).
50. Luca, T. et al. Axons mediate the distribution of arylsulfatase A within the mouse hippocampus upon gene delivery. Mol Ther 12, 669-79 (2005).
51. Passini, M. A., Lee, E. B., Heuer, G. G. & Wolfe, J. H. Distribution of a lysosomal enzyme in the adult brain by axonal transport and by cells of the rostral migratory stream. J Neurosci 22, 6437-46 (2002).
52. Passini, M. A. et al. AAV vector-mediated correction of brain pathology in a mouse model of Niemann-Pick A disease. Mol Ther 11, 754-62 (2005).
53. Griffey, M., Macauley, S. L., Ogilvie, J. M. & Sands, M. S. AAV2-mediated ocular gene therapy for infantile neuronal ceroid lipofuscinosis. Mol Ther 12, 413-21 (2005).
54. Hennig, A. K. et al. AAV-mediated intravitreal gene therapy reduces lysosomal storage in the retinal pigmented epithelium and improves retinal function in adult MPS VII mice. Mol Ther 10, 106-16 (2004).
55. Liu, G., Martins, I., Wemmie, J. A., Chiorini, J. A. & Davidson, B. L. Functional correction of CNS phenotypes in a lysosomal storage disease model using adeno-associated virus type 4 vectors. J Neurosci 25, 9321-7 (2005).
56. Skorupa, A. F., Fisher, K. J., Wilson, J. M., Parente, M. K. & Wolfe, J. H. Sustained production of beta-glucuronidase from localized sites after AAV vector gene transfer results in widespread distribution of enzyme and reversal of lysosomal storage lesions in a large volume of brain in mucopolysaccharidosis VII mice. Exp Neurol 160, 17-27 (1999).
57. Bosch, A., Perret, E., Desmaris, N. & Heard, J. M. Long-term and significant correction of brain lesions in adult mucopolysaccharidosis type VII mice using recombinant AAV vectors. Mol Ther 1, 63-70 (2000).
58. Sferra, T. J. et al. Recombinant adeno-associated virus-mediated correction of lysosomal storage within the central nervous system of the adult mucopolysaccharidosis type VII mouse. Hum Gene Ther 11, 507-19 (2000).
59. Fu, H. et al. Neurological correction of lysosomal storage in a mucopolysaccharidosis IIIB mouse model by adeno-associated virus-mediated gene delivery. Mol Ther 5, 42-9 (2002).
60. Haskell, R. E., Hughes, S. M., Chiorini, J. A., Alisky, J. M. & Davidson, B. L. Viral-mediated delivery of the late-infantile neuronal ceroid lipofuscinosis gene, TPP-I to the mouse central nervous system. Gene Ther 10, 34-42 (2003).
61. Desmaris, N. et al. Prevention of neuropathology in the mouse model of Hurler syndrome. Ann Neurol 56, 68-76 (2004).
62. Cressant, A. et al. Improved behavior and neuropathology in the mouse model of Sanfilippo type IIIB disease after adeno-associated virus-mediated gene transfer in the striatum. J Neurosci 24, 10229-39 (2004).
63. Passini, M. A. et al. Intracranial delivery of CLN2 reduces brain pathology in a mouse model of classical late infantile neuronal ceroid lipofuscinosis. J Neurosci 26, 1334-42 (2006).
64. Groenewegen, H. J. & Witter, M. P. Thalamus. in The Rat Nervous System (ed. Paxinos, G.) 407-453 (Elsevier Academic Press, San Diego, 2004).
65. Cachon-Gonzalez, M. B. et al. Effective gene therapy in an authentic model of Tay-Sachs-related diseases. Proceedings of the National Academy of Sciences of the United States of America 103, 10373-10378 (2006).
66. Orii, K. O. et al. Defining the pathway for Tat-mediated delivery of beta-glucuronidase in cultured cells and MPS VII mice. Mol Ther 12, 345-52 (2005).
67. Xia, H., Mao, Q. & Davidson, B. L. The HIV Tat protein transduction domain improves the biodistribution of beta-glucuronidase expressed from recombinant viral vectors. Nat Biotechnol 19, 640-4 (2001).
68. Broekman, M. L., Corner, L. A., Hyman, B. T. & Sena-Esteves, M. Adeno-associated virus vectors serotyped with AAVrh.8 capsid are more efficient than AAV-1 or -2 serotypes for widespread gene delivery to the neonatal mouse brain. Neuroscience 138, 501-10 (2006).
69. Burger, C. et al. Recombinant AAV viral vectors pseudotyped with viral capsids from serotypes 1, 2, and 5 display differential efficiency and cell tropism after delivery to different regions of the central nervous system. Mol Ther 10, 302-17 (2004).
70. Cabrera-Salazar, M. A. et al. Timing of therapeutic intervention determines functional and survival outcomes in a mouse model of late infantile batten disease. Mol Ther 15, 1782-8 (2007).
71. Dodge, J. C. et al. Gene transfer of human acid sphingomyelinase corrects neuropathology and motor deficits in a mouse model of Niemann-Pick type A disease. Proc Natl Acad Sci USA 102, 17822-7 (2005).
72. Klein, R. L. et al. Efficient neuronal gene transfer with AAVrh.8 leads to neurotoxic levels of tau or green fluorescent proteins. Mol Ther 13, 517-27 (2006).
73. Passini, M. A. et al. Intraventricular brain injection of adeno-associated virus type 1 (AAV1) in neonatal mice results in complementary patterns of neuronal transduction to AAV2 and total long-term correction of storage lesions in the brains of beta-glucuronidase-deficient mice. J Virol 77, 7034-40 (2003).
74. Wang, C., Wang, C. M., Clark, K. R. & Sferra, T. J. Recombinant AAV serotype 1 transduction efficiency and tropism in the murine brain. Gene Ther 10, 1528-34 (2003).
75. Vite, C. H., Passini, M. A., Haskins, M. E. & Wolfe, J. H. Adeno-associated virus vector-mediated transduction in the cat brain. Gene Ther 10, 1874-81 (2003).
76. Passini, M. A. et al. Combination brain and systemic injections of AAV provide maximal functional and survival benefits in the Niemann-Pick mouse. Proc Natl Acad Sci USA 104, 9505-10 (2007).
77. Jeyakumar, M. et al. Central nervous system inflammation is a hallmark of pathogenesis in mouse models of GM1 and GM2 gangliosidosis. Brain 126, 974-87 (2003).
78. Wada, R., Tifft, C. J. & Proia, R. L. Microglial activation precedes acute neurodegeneration in Sandhoff disease and is suppressed by bone marrow transplantation. Proc Natl Acad Sci USA 97, 10954-9 (2000).
79. Jeyakumar, M. et al. Delayed symptom onset and increased life expectancy in Sandhoff disease mice treated with N-butyldeoxynojirimycin. Proc Natl Acad Sci USA 96, 6388-93 (1999).
80. Norflus, F. et al. Bone marrow transplantation prolongs life span and ameliorates neurologic manifestations in Sandhoff disease mice. J Clin Invest 101, 1881-8 (1998).
81. Yamaguchi, A. et al. Possible role of autoantibodies in the pathophysiology of GM2 gangliosidoses. J Clin Invest 113, 200-8 (2004).
82. Watson, G. et al. Intrathecal administration of AAV vectors for the treatment of lysosomal storage in the brains of MPS I mice. Gene Ther 13, 917-25 (2006).
83. Donsante, A. et al. AAV vector integration sites in mouse hepatocellular carcinoma. Science 317, 477 (2007).
84. Brain tumors in man and animals: report of a workshop. Environ Health Perspect 68, 155-73 (1986). 8
85. Denny, C. A. et al. Neurochemical, morphological, and neurophysiological abnormalities in retinas of Sandhoff and GM1 gangliosidosis mice. J Neurochem 101, 1294-302 (2007).
86. Klein, R. L. et al. Dose and promoter effects of adeno-associated viral vector for green fluorescent protein expression in the rat brain. Exp Neurol 176, 66-74 (2002).
87. Luo, J. et al. Subthalamic GAD gene therapy in a Parkinson's disease rat model. Science 298, 425-9 (2002).
88. Donello, J. E., Loeb, J. E. & Hope, T. J. Woodchuck hepatitis virus contains a tripartite posttranscriptional regulatory element. J Virol 72, 5085-92 (1998).
89. Lacorazza, H. D. & Jendoubi, M. In situ assessment of beta-hexosaminidase activity. Biotechniques 19, 434-40 (1995).
90. Passini, M. A. & Wolfe, J. H. Widespread gene delivery and structure-specific patterns of expression in the brain after intraventricular injections of neonatal mice with an adeno-associated virus vector. J Virol 75, 12382-92 (2001).
91. Denny, C. A., Kasperzyk, J. L., Gorham, K. N., Bronson, R. T. & Seyfried, T. N. Influence of caloric restriction on motor behavior, longevity, and brain lipid composition in Sandhoff disease mice. J Neurosci Res 83, 1028-38 (2006).
92. Kasperzyk, J. L. et al. N-butyldeoxygalactonojirimycin reduces neonatal brain ganglioside content in a mouse model of GM1 gangliosidosis. J Neurochem 89, 645-53 (2004).
93. Hauser, E. C., Kasperzyk, J. L., d'Azzo, A. & Seyfried, T. N. Inheritance of lysosomal acid b-galactosidase activity and gangliosides in crosses of DBA/2J and knockout mice. Biochem. Genetics 42, 241-257 (2004).
94. Cambron, L. D. & Leskawa, K. C. A sensitive method to quantitate gangliosides of the gangliotetraose series directly on chromatograms using peroxidase conjugated cholera toxin. Stain Technol 65, 293-7 (1990).
95. Kasperzyk, J. L., d'Azzo, A., Platt, F. M., Alroy, J. & Seyfried, T. N. Substrate reduction therapy reduces ganglioside content in postnatal cerebrum-brainstem and cerebellum in a mouse model of GM1 gangliosidosis. J Lipid Res (2005).
96. Yu, R. K. & Ledeen, R. W. Gas—liquid chromatographic assay of lipid-bound sialic acids: measurement of gangliosides in brain of several species. J. Lipid Res. 11, 506-516 (1970).
97. Seyfried, T. N., Novikov, A. M., Irvine, R. A. & Brigande, J. V. Ganglioside biosynthesis in mouse embryos: sialyltransferase IV and the asialo pathway. J. Lipid Res. 35, 993-1001 (1994).
98. Bai, H. & Seyfried, T. N. Influence of ganglioside GM3 and high density lipoprotein (HDL) on the cohesion of mouse brain tumor cells. J. Lipid Res. 38, 160-172. (1997).
99. Ingram, D. K., London, E. D., Reynolds, M. A., Waller, S. B. & Goodrick, C. L. Differential effects of age on motor performance in two mouse strains. Neurobiol Aging 2, 221-7 (1981).
100. Holland, H. C. & Weldon, E. A note on a new technique of recording ambulation in the open field test and its validation. Acta Psychol (Amst) 28, 293-300 (1968).
101. Barnes, C. A. Memory deficits associated with senescence: a neurophysiological and behavioral study in the rat. J Comp Physiol Psychol 93, 74-104 (1979).
102. Tumosa, N. & Baker, J. R. Microglia in the nerve fiber layer of the cat retina: detection of postnatal changes by a new monoclonal antibody. Vis.Neurosci. 13, 671-682 (1996).
103. Tumosa, N. & Baker, J. R. The monoclonal antibody H386F labels microglia in the retinal nerve fiber layer of several mammals. Vis.Neurosci. 14, 663-669 (1997).
104. Kroll, R. A. et al. White matter changes associated with feline GM2 gangliosidosis (Sandhoff disease): correlation of MR findings with pathologic and ultrastructural abnormalities. AJNR Am J Neuroradiol 16, 1219-26 (1995).
105. Walkley, S. U., Baker, H. J. & Rattazzi, M. C. Initiation and growth of ectopic neurites and meganeurites during postnatal cortical development in ganglioside storage disease. Brain Res Dev Brain Res 51, 167-78 (1990).
106. Jeyakumar, M. et al. NSAIDs increase survival in the Sandhoff Disease mouse: synergy with N-butyldeoxynojirimycin. Ann Neurol 56, 642-649 (2004).
107. Hasegawa, D. et al. Clinical and molecular analysis of GM2 gangliosidosis in two apparent littermate kittens of the Japanese domestic cat. J Feline Med Surg 9, 232-7 (2007).
108. Yamato, O. et al. Laboratory diagnosis of canine GM2-gangliosidosis using blood and cerebrospinal fluid. J Vet Diagn Invest 16, 39-44 (2004).
109. Ponder, K. P. et al. Mucopolysaccharidosis I cats mount a cytotoxic T lymphocyte response after neonatal gene therapy that can be blocked with CTLA4-Ig. Mol Ther 14, 5-13 (2006).
110. Abrams, J. R. et al. CTLA4Ig-mediated blockade of T-cell costimulation in patients with psoriasis vulgaris. J Clin Invest 103, 1243-52 (1999).
111. Viglietta, V. et al. CTLA4Ig treatment in patients with multiple sclerosis: an open-label, phase 1 clinical trial. Neurology 71, 917-24 (2008).
112. Bankiewicz, K. S. et al. Long-term clinical improvement in MPTP-lesioned primates after gene therapy with AAV-hAADC. Mol Ther 14, 564-70 (2006).
113. Fiandaca, M., Forsayeth, J. & Bankiewicz, K. Current status of gene therapy trials for Parkinson's disease. Exp Neurol 209, 51-57 (2008).
114. Wooldridge, J. D., Gregory, C. R., Mathews, K. G., Aronson, L. R. & Kyles, A. E. The prevalence of malignant neoplasia in feline renal-transplant recipients. Vet Surg 31, 94-7 (2002).
115. Harding, T. C. et al. Enhanced gene transfer efficiency in the murine striatum and an orthotopic glioblastoma tumor model, using AAV-7- and AAV-8-pseudotyped vectors. Hum Gene Ther 17, 807-20 (2006).
116. Baek, R C, et al. AAV-mediated gene delivery in adult GM1-gangliosidosis mice corrects lysosomal storage in CNS and improves survival. PLosOne 5(10), e13468 (2010).
117. Maguire, C A et al. Preventing growth of brain tumors by creating a zone of resistance. Mol. Ther. 16(10); 1695-1702 (2008).

1. A method for enhancing β-N-acetylhexosaminidase activity in a subject in need thereof, comprising:
administering to the subject a therapeutically effective amount of a composition comprising a first expression construct and a second expression construct, wherein
(i) the first construct expresses the β-N-acetylhexosaminidase β subunit; and
(ii) the second construct expresses the β-N-acetylhexosaminidase α subunit;
wherein the composition is administered to at least two or more brain areas of the subject, the brain areas selected from the group consisting of thalamus, striatum, deep cerebellar nuclei, and ventral tegmental area.

2. The method of claim 1, wherein the subject is a human predisposed to having, suspected of having, or diagnosed as having a β-N-acetylhexosaminidase deficiency.

3. The method of claim 2, wherein the β-N-acetylhexosaminidase deficiency comprises a lysosomal storage disorder.

4. The method of claim 2, wherein the β-N-acetylhexosaminidase deficiency comprises Tay-Sachs Disease or Sandhoff Disease.

5. The method of claim 2, wherein the β-N-acetylhexosaminidase deficiency comprises a partial or complete loss of endogenous expression or function of the β-N-acetylhexosaminidase subunit, β subunit, α subunit, or both.

6. The method of claim 1, wherein the expression constructs comprise the adeno-associated virus (AAV) vector AAVrh.8 or derivatives thereof.

7-8. (canceled)

9. The method of claim 1, comprising administering the composition to the brain of the subject unilaterally or bilaterally.

10. The method of claim 1, comprising evaluating β-N-acetylhexosaminidase activity in the subject after administration of the composition, wherein evaluating β-N-acetylhexosaminidase activity comprises an assessment of symptoms associated with a β-N-acetylhexosaminidase deficiency or a biochemical assessment of β-N-acetylhexosaminidase activity.

11-12. (canceled)

13. The method of claim 1, comprising administering additional amounts of the composition to the subject as needed to achieve or maintain enhanced β-N-acetylhexosaminidase activity.

14-35. (canceled)

36. A method for achieving efficient and high rate of expression of exogenous β-N-acetylhexosaminidase in the brain of a subject in need thereof, comprising:
administering to the subject a therapeutically effective amount of a composition comprising a first expression construct and a second expression construct, wherein
(i) the first construct expresses the β-N-acetylhexosaminidase β subunit; and
(ii) the second construct expresses the β-N-acetylhexosaminidase α subunit;
wherein the composition is administered to at least two or more brain areas of the subject, the brain areas selected from the group consisting of thalamus, striatum, deep cerebellar nuclei, and ventral tegmental area.

37. The method of claim 36, wherein the subject is a human predisposed to having, suspected of having, or diagnosed as having a β-N-acetylhexosaminidase deficiency.

38. The method of claim 37, wherein the β-N-acetylhexosaminidase deficiency comprises a disorder or a disease selected from the group consisting of a lysosomal storage disorder, Tay-Sachs Disease and Sandhoff Disease.

39-40. (canceled)

41. The method of claim 36, wherein the expression constructs comprise the adeno-associated virus (AAV) vector AAVrh.8 or derivatives thereof.

42. (canceled)

43. The method of claim 36, comprising administering the composition to the brain of the subject unilaterally or bilaterally.

44. The method of claim 36, comprising evaluating β-N-acetylhexosaminidase activity in the subject after administration of the composition, wherein evaluating β-N-acetylhexosaminidase activity comprises an assessment of symptoms associated with a β-N-acetylhexosaminidase deficiency or a biochemical assessment of β-N-acetylhexosaminidase activity.

45-46. (canceled)

47. The method of claim 36, comprising administering additional amounts of the composition to the subject as needed to achieve or maintain elevated expression of exogenous β-N-acetylhexosaminidase activity in the brain of the subject.

48-85. (canceled)

86. A method for achieving efficient and high rate of expression of exogenous acid β-galactosidase in the brain of a subject in need thereof, comprising: administering to the subject a therapeutically effective amount of a composition comprising an acid β-galactosidase expression construct, wherein the composition is administered to two or more brain areas selected from the group consisting of thalamus, striatum, deep cerebellar nuclei, and ventral tegmental area.

87. The method of claim 86, wherein the subject is a human predisposed to having, suspected of having, or diagnosed as having an acid β-galactosidase deficiency, wherein the acid β-galactosidase deficiency comprises a partial or complete loss of endogenous expression or function of acid β-galactosidase.

88. The method of claim 87, wherein the acid β-galactosidase deficiency comprises a lysosomal storage disorder.

89. The method of claim 87, wherein the acid β-galactosidase deficiency comprises GM1-gangliosidosis, wherein the GM1-gangliosidosis comprises a partial or complete loss of endogenous expression or function of acid β-galactosidase.

90. (canceled)

91. The method of claim 86, wherein the expression construct comprises the adeno-associated virus (AAV) vector AAVrh.8 or derivatives thereof.

92. (canceled)

93. The method of claim 86, comprising administering the composition to the brain of the subject unilaterally or bilaterally.

94. The method of claim 86, comprising evaluating acid β-galactosidase activity in the subject after administration of the composition, wherein evaluating acid β-galactosidase activity comprises an assessment of symptoms associated with the acid β-galactosidase deficiency or a biochemical assessment of acid β-galactosidase activity.

95-96. (canceled)

97. The method of claim 86, comprising administering additional amounts of the composition to the subject as needed to achieve or maintain a high rate of expression of exogenous acid β-galactosidase activity in the brain of the subject.

98-100. (canceled)

101. The methods of claim 1, comprising administering the composition to three or more areas of the brain of the subject selected from the group consisting of thalamus, striatum, deep cerebellar nuclei, and ventral tegmental area.

102. The method of claim 101, comprising administering the composition bilaterally into the thalamus and unilaterally into the deep cerebellar nuclei.

103. The methods of claim 36, comprising administering the composition to three or more areas of the brain of the subject selected from the group consisting of thalamus, striatum, deep cerebellar nuclei, and ventral tegmental area.

104. The method of claim 103, comprising administering the composition bilaterally into the thalamus and unilaterally into the deep cerebellar nuclei.

105. The methods of claim 86, comprising administering the composition to three or more areas of the brain of the subject selected from the group consisting of thalamus, striatum, deep cerebellar nuclei, and ventral tegmental area.

106. The method of claim 105, comprising administering the composition bilaterally into the thalamus and unilaterally into the deep cerebellar nuclei.

107. The method of claim 1, wherein the composition is administered via cerebral spinal fluid (CSF).

108. The method of claim 107, wherein the composition is administered to CSF via the lateral ventricles or perivascular space of Virchow-Robin.

109. The method of claim 36, wherein the composition is administered via cerebral spinal fluid (CSF).

110. The method of claim 109, wherein the composition is administered to CSF via the lateral ventricles or perivascular space of Virchow-Robin.

111. The method of claim 86, wherein the composition is administered via cerebral spinal fluid (CSF).

112. The method of claim 111, wherein the composition is administered to CSF via the lateral ventricles or perivascular space of Virchow-Robin.
